(19) United States
(12) Patent Application Publication
(10) Pub. No.: US 2023/0272118 A1
(43) Pub. Date: Aug. 31, 2023

(54) Dosing Regimens for Mitigation of Cytokine Release Syndrome

(71) Applicant: Regeneron Pharmaceuticals, Inc, Tarrytown, NY (US)

(72) Inventors: Srikanth R. Ambati, Rye Brook, NY (US); Aafia Chaudhry, New Rochelle, NY (US); Hesham Mohamed, Princeton, NJ (US); Masood Khaksar Toroghi, Congers, NY (US); Min Zhu, Ossining, NY (US)

(21) Appl. No.: 18/114,057

(22) Filed: Feb. 24, 2023

Related U.S. Application Data
(60) Provisional application No. 63/313,932, filed on Feb. 25, 2022.
(60) Provisional application No. 63/393,212, filed on Jul. 28, 2022.
(60) Provisional application No. 63/420,782, filed on Oct. 31, 2022.
(60) Provisional application No. 63/424,210, filed on Nov. 10, 2022.
(60) Provisional application No. 63/440,304, filed on Jan. 20, 2023.

(51) Int. Cl. C07K 16/46; A61P 35/00; C07K 16/28

(52) U.S. Cl. CPC C07K 16/468; A61P 35/00; C07K 16/2866; C07K 2317/565; C07K 2317/31

(57) ABSTRACT

Administration regimens for anti-CD3 x anti-CD20 bispecific antibodies that mitigate cytokine release syndrome and infusion-related reaction are disclosed. The methods employ fractional dosing over several weeks of treatment along with administration of a steroid and an antihistamine.

### CROSS-REFERENCE TO RELATED APPLICATIONS

This application claims the benefit under 35 USC § 119(e) of U.S. Provisional Application Nos. 63/313,932, filed Feb. 25, 2022; 63/393,212, filed Jul. 28, 2022; 63/420,782, filed Oct. 31, 2022; 63/424,210, filed Nov. 10, 2022; and 63/440,304, filed Jan. 20, 2023, each of which is incorporated herein by reference in its entirety for all purposes.

### REFERENCE TO A SEQUENCE LISTING

This application incorporates by reference a computer readable Sequence Listing in ST.26 XML format, titled 11150US01_Sequence, created on Feb. 24, 2023 and containing 26,237 bytes.

### FIELD OF THE INVENTION

The present invention lies in the field of medicine, and relates to dosing regimens for a bispecific anti-CD3 x anti-CD20 antibody that mitigate the prevalence and severity of cytokine release syndrome or an infusion-related reaction in patients undergoing immunotherapy.

### BACKGROUND

Cytokine release syndrome (CRS) is a systemic inflammatory response that can be triggered by a variety of factors, including certain drugs. T cell-activating cancer immunotherapies carry a particularly high risk of CRS, which is usually due to on-target effects induced by binding of a bispecific antibody or chimeric antigen receptor (CAR) T cell to its antigen and subsequent activation of bystander immune cells and non-immune cells, such as endothelial cells. Activation of the bystander cells results in the massive release of a range of cytokines. IL-6, IL-10, and interferon (IFN)-γ are among the core cytokines that are consistently found to be elevated in serum of patients with CRS. With T cell-activating therapies directed against tumor cells, CRS is triggered by the massive release of IFN-γ by activated T cells or the tumor cells themselves. Secreted IFN-γ induces activation of other immune cells, most importantly macrophages, which in turn produce excessive amounts of additional cytokines such as IL-6, TNF-α, and IL-10. IL-6, in particular, contributes to many of the key symptoms of CRS, including vascular leakage, and activation of the complement and coagulation cascade inducing disseminated intravascular coagulation. In addition, IL-6 likely contributes to cardiomyopathy by promoting myocardial dysfunction. Shimabukaro-Vornhagen et al., Journal for Immunotherapy of Cancer, 6:56, pp. 1-14, 2018. In some cases, the symptoms associated with CRS are termed infusion-related reaction (IRR) if they occur less than six hours following the start of infusion, and CRS if they occur from six hours onward following the start of infusion.

The management of the toxicities of cancer immunotherapy is a challenging clinical problem. Mitigating CRS or IRR is a hallmark of administering certain treatment modalities, for example CAR T cells and bispecific antibodies targeting T cells. Low grade CRS is generally treated symptomatically with anti-histamines, antipyretics and fluids. Severe CRS can represent a life-threatening adverse event that requires prompt and aggressive treatment. Reduction of tumor burden, limitations on the dose of administered therapy, and premedication with steroids have reduced the incidence of severe CRS, as have the use of anti-cytokine treatments. Tocilizumab, an anti-IL-6 antibody, has become a standard initial treatment for severe CRS in some circumstances. There remains a need for alternative strategies to mitigate the potential life-threatening effects of CRS without negatively impacting the therapeutic benefits of immunotherapies.

### BRIEF SUMMARY OF THE INVENTION

In one aspect, the present disclosure provides a dosing regimen for administering an anti-CD3 x anti-CD20 bispecific antibody to a subject to treat a B-cell malignancy, comprising: (a) administering an initial dose of 0.7 mg of the bispecific antibody to the subject, wherein the initial dose is split into a first dose fraction comprising 0.2 mg of the bispecific antibody and a second dose fraction comprising 0.5 mg of the bispecific antibody, wherein the first dose fraction is administered to the subject followed by the second dose fraction over two days during week 1 of the dosing regimen; (b) administering a first intermediate dose of 4 mg of the bispecific antibody to the subject, wherein the first intermediate dose is split into two equal fractions (a first fraction and a second fraction), each comprising 2 mg of the bispecific antibody, wherein the two fractions of the first intermediate dose are administered over two days during week 2 of the dosing regimen; (c) administering a second intermediate dose of 20 mg of the bispecific antibody to the subject, wherein the second intermediate dose is split into two equal fractions (a first fraction and a second fraction), each comprising 10 mg of the bispecific antibody, wherein the two fractions of the second intermediate dose are administered over two days during week 3 of the dosing regimen; (d) administering a full dose of the bispecific antibody to the subject weekly during weeks 4 to 12 of the dosing regimen; and (e) administering a maintenance dose of the bispecific antibody to the subject in week 14 of the dosing regimen, wherein the bispecific antibody comprises a first antigen-binding region that binds human CD20 and a second antigen-binding region that binds human CD3, wherein the first antigen-binding region comprises three heavy chain complementarity determining regions, HCDR1, HCDR2 and HCDR3 comprising the amino acid sequences of SEQ ID NO: 7, 8 and 9, respectively, and three light chain complementarity determining regions LCDR1, LCDR2 and LCDR3 comprising the amino acid sequences of SEQ ID NO: 13, 14 and 15, respectively, and wherein the second antigen-binding region comprises three heavy chain complementarity determining regions, HCDR1, HCDR2 and HCDR3 comprising the amino acid sequences of SEQ ID NO: 10, 11 and 12, respectively, and three light chain complementarity determining regions LCDR1, LCDR2 and LCDR3 comprising the amino acid sequences of SEQ ID NO: 13, 14 and 15, respectively. In some embodiments, the initial dose (0.7 mg) may be split into other fractions, such as 0.1 mg and 0.6 mg, 0.15 mg and 0.55 mg, 0.25 mg and 0.45 mg, or 0.3 mg and 0.4 mg.

In some embodiments, the full dose is administered to the subject as a single dose during weeks 4 to 12 of the dosing regimen.

In some embodiments, if the subject experiences a grade 3 event of cytokine release syndrome when administered the initial dose, the first intermediate dose, or the second intermediate dose, then the full dose of the bispecific antibody administered to the subject during week 4 is split into two equal fractions and the two fractions of the full dose are administered over two days during week 4 of the dosing regimen, and wherein the full dose is administered to the subject as a single dose during weeks 5 to 12 of the dosing regimen.

In some embodiments, the maintenance dose is administered to the subject every two weeks beginning in week 14 of the dosing regimen.

In some embodiments, the maintenance dose is administered to the subject every four weeks or eight weeks beginning in a subsequent week of the dosing regimen, wherein the subsequent week is at least week 36 of the dosing regimen.

In some embodiments, the second dose fraction of the initial dose is administered to the subject from 18 to 96 hours after the first dose fraction of the initial dose. In some embodiments, the two fractions of the first intermediate dose are administered to the subject from 18 to 96 hours apart. In some embodiments, the two fractions of the second intermediate dose are administered to the subject from 18 to 96 hours apart. In some embodiments, the two fractions of the full dose are administered to the subject from 18 to 96 hours apart.

In some embodiments, the two days are consecutive days. In some embodiments, the two days are no more than three days apart.

In some embodiments, the B-cell malignancy is a B-cell non-Hodgkin lymphoma. In some cases, the B-cell malignancy is follicular lymphoma, diffuse large B-cell lymphoma, mantle cell lymphoma, or marginal zone lymphoma.

In some embodiments, the full dose of the bispecific antibody is from 80 mg to 320 mg.

In some embodiments, the maintenance dose of the bispecific antibody is from 160 mg to 320 mg.

In some embodiments, the B-cell malignancy is follicular lymphoma, the full dose is 80 mg and the maintenance dose is 160 mg or 320 mg. In some cases, the follicular lymphoma is grade 1-3a.

In some embodiments, the B-cell malignancy is diffuse large B-cell lymphoma, the full dose is 160 mg and the maintenance dose is 320 mg. In some embodiments, the B-cell malignancy is diffuse large B-cell lymphoma, the full dose is 320 mg and the maintenance dose is 320 mg. In some cases, the subject has failed prior CAR-T therapy.

In some embodiments, the B-cell malignancy is mantle cell lymphoma, the full dose is 160 mg and the maintenance dose is 320 mg. In some cases, the subject has failed prior Bruton tyrosine kinase (BTK) inhibitor therapy.

In some embodiments, the B-cell malignancy is marginal zone lymphoma, the full dose is 80 mg and the maintenance dose is 160 mg.

In some embodiments, the B-cell malignancy is a non-Hodgkin lymphoma other than follicular lymphoma, diffuse large B-cell lymphoma, mantle cell lymphoma, or marginal zone lymphoma, the full dose is 160 mg and the maintenance dose is 320 mg.

In some embodiments, the B-cell malignancy is an aggressive lymphoma, the full dose is 160 mg and the maintenance dose is 320 mg.

In some embodiments, the subject has documented CD20+ B-cell malignancy, with active disease not responsive to prior therapy, for whom no standard of care options exists, and for whom treatment with an anti-CD20 antibody may be appropriate. In some embodiments, the subject has documented aggressive B-NHL, has relapsed within one year of frontline therapy and intends to proceed with autologous stem cell transplant (ASCT).

In some embodiments, the subject is previously untreated with a systemic anti-lymphoma therapy. In some embodiments, the subject has relapsed or refractory disease.

In some embodiments, the subject has relapsed or is refractory to at least 2 prior lines of systemic therapy, including an anti-CD20 antibody and an alkylating agent.

In some embodiments, the subject is refractory to an anti-CD20 antibody in any line of prior therapy.

In some embodiments, the subject is double refractory to an alkylating agent and an anti-CD20 antibody in any line of prior therapy.

In some embodiments, the subject is a human aged 18 years.

In any of the various embodiments discussed in the above aspect of the disclosure, the bispecific antibody may be administered intravenously.

In one aspect, the present disclosure provides a dosing regimen for administering an anti-CD3 x anti-CD20 bispecific antibody to a subject to treat a B-cell malignancy, comprising: administering an initial dose of 1 mg or 2 mg of the bispecific antibody to the subject during week 1 of the dosing regimen; administering a first intermediate dose of 10 mg or 26 mg of the bispecific antibody to the subject during week 2 of the dosing regimen; administering a second intermediate dose of 50 mg or 100 mg of the bispecific antibody to the subject during week 3 of the dosing regimen; and administering a full dose of the bispecific antibody to the subject during week 4 and during a subsequent week of the dosing regimen, wherein the bispecific antibody comprises a first antigen-binding region that binds human CD20 and a second antigen-binding region that binds human CD3, wherein the first antigen-binding region comprises three heavy chain complementarity determining regions, HCDR1, HCDR2 and HCDR3 comprising the amino acid sequences of SEQ ID NO: 7, 8 and 9, respectively, and three light chain complementarity determining regions LCDR1, LCDR2 and LCDR3 comprising the amino acid sequences of SEQ ID NO: 13, 14 and 15, respectively, and wherein the second antigen-binding region comprises three heavy chain complementarity determining regions, HCDR1, HCDR2 and HCDR3 comprising the amino acid sequences of SEQ ID NO: 10, 11 and 12, respectively, and three light chain complementarity determining regions LCDR1, LCDR2 and LCDR3 comprising the amino acid sequences of SEQ ID NO: 13, 14 and 15, respectively.

In some embodiments, the initial dose is 2 mg. In some embodiments, the first intermediate dose is 26 mg. In some embodiments, the second intermediate dose is 100 mg. In some embodiments, the full dose is 200 mg, 400 mg or 600 mg. In some cases, the full dose is 400 mg. In some cases, the full dose is 600 mg.

In some embodiments, the full dose is administered to the subject once every three weeks. In some embodiments, the full dose is administered to the subject weekly. In some cases, the full dose is administered to the subject weekly for three weeks, and then the full dose is administered to the subject once every three weeks.

In some embodiments, the subject has relapsed or refractory disease. In some embodiments, the subject is refractory to an anti-CD20 antibody in any line of prior therapy.

In some embodiments, the B-cell malignancy is follicular lymphoma. In some embodiments, the B-cell malignancy is diffuse large B-cell lymphoma.

In some embodiments, the subject is a human aged 18 years.

In any of the various embodiments discussed in the above aspect of the disclosure, the bispecific antibody may be administered subcutaneously.

In one aspect, the present disclosure provides a dosing regimen for administering an anti-CD3 x anti-CD20 bispecific antibody to a subject to treat a B-cell malignancy, comprising: administering an initial dose of the bispecific antibody to the subject, wherein the initial dose is split into a first dose fraction of the bispecific antibody and a second dose fraction of the bispecific antibody, wherein the first dose fraction is administered to the subject followed by the second dose fraction over two days during week 1 of the dosing regimen; administering a first intermediate dose of the bispecific antibody to the subject, wherein the first intermediate dose is split into two equal fractions (a first fraction and a second fraction) of the bispecific antibody, wherein the two fractions of the first intermediate dose are administered over two days during week 2 of the dosing regimen; administering a second intermediate dose of the bispecific antibody to the subject, wherein the second intermediate dose is split into two equal fractions (a first fraction and a second fraction) of the bispecific antibody, wherein the two fractions of the second intermediate dose are administered over two days during week 3 of the dosing regimen; administering a full dose of the bispecific antibody to the subject weekly during weeks 4 to 12 of the dosing regimen; and administering a maintenance dose of the bispecific antibody to the subject in week 14 of the dosing regimen, wherein the initial dose, the first intermediate dose, the second intermediate dose, the full dose and the maintenance dose is dependent on the weight of subject: (i) if the subject has a weight of from 40 kg to less than 165 kg (e.g., 40-164 kg), the initial dose is 0.7 mg, the first dose fraction of the initial dose is 0.2 mg, the second dose fraction of the initial dose is 0.5 mg, the first intermediate dose is 4 mg, the two equal fractions of the first intermediate dose each comprise 2 mg, the second intermediate dose is 20 mg, the two equal fractions of the second intermediate dose each comprise 10 mg, the full dose is 160 mg, and the maintenance dose is 320 mg; (ii) if the subject has a weight of from 20 kg to less than 40 kg (e.g., 20-39 kg), the initial dose is 0.4 mg, the first dose fraction of the initial dose is 0.1 mg, the second dose fraction of the initial dose is 0.3 mg, the first intermediate dose is 2 mg, the two equal fractions of the first intermediate dose each comprise 1 mg, the second intermediate dose is 12 mg, the two equal fractions of the second intermediate dose each comprise 6 mg, the full dose is 90 mg, and the maintenance dose is 150 mg; (iii) if the subject has a weight of from 10 kg to less than 20 kg (e.g., 10-19 kg), the initial dose is 0.3 mg, the first dose fraction of the initial dose is 0.1 mg, the second dose fraction of the initial dose is 0.2 mg, the first intermediate dose is 1.6 mg, the two equal fractions of the first intermediate dose each comprise 0.8 mg, the second intermediate dose is 8 mg, the two equal fractions of the second intermediate dose each comprise 4 mg, the full dose is 60 mg, and the maintenance dose is 100 mg; or (iv) if the subject has a weight of from 6 kg to less than 10 kg (e.g., 6-9 kg), the initial dose is 0.24 mg, the first dose fraction of the initial dose is 0.07 mg, the second dose fraction of the initial dose is 0.17 mg, the first intermediate dose is 1.2 mg, the two equal fractions of the first intermediate dose each comprise 0.6 mg, the second intermediate dose is 6 mg, the two equal fractions of the second intermediate dose each comprise 3 mg, the full dose is 45 mg, and the maintenance dose is 75 mg; and wherein the bispecific antibody comprises a first antigen-binding region that binds human CD20 and a second antigen-binding region that binds human CD3, wherein the first antigen-binding region comprises three heavy chain complementarity determining regions, HCDR1, HCDR2 and HCDR3 comprising the amino acid sequences of SEQ ID NO: 7, 8 and 9, respectively, and three light chain complementarity determining regions LCDR1, LCDR2 and LCDR3 comprising the amino acid sequences of SEQ ID NO: 13, 14 and 15, respectively, and wherein the second antigen-binding region comprises three heavy chain complementarity determining regions, HCDR1, HCDR2 and HCDR3 comprising the amino acid sequences of SEQ ID NO: 10, 11 and 12, respectively, and three light chain complementarity determining regions LCDR1, LCDR2 and LCDR3 comprising the amino acid sequences of SEQ ID NO: 13, 14 and 15, respectively.

In some embodiments, the full dose is administered to the subject as a single dose during weeks 4 to 12 of the dosing regimen. In some embodiments, the maintenance dose is administered to the subject every two weeks beginning in week 14 of the dosing regimen.

In some embodiments, the second dose fraction of the initial dose is administered to the subject from 18 to 96 hours after the first dose fraction of the initial dose. In some embodiments, the two fractions of the first intermediate dose are administered to the subject from 18 to 96 hours apart. In some embodiments, the two fractions of the second intermediate dose are administered to the subject from 18 to 96 hours apart.

In some embodiments, the two days are consecutive days. In some embodiments, the two days are no more than three days apart.

In some embodiments, the B-cell malignancy is a B-cell non-Hodgkin lymphoma. In some embodiments, the B-cell malignancy is follicular lymphoma, diffuse large B-cell lymphoma, mantle cell lymphoma, or marginal zone lymphoma.

In some embodiments, the subject is a human aged<18 years.

In any of the various embodiments discussed in the above aspect of the disclosure, the bispecific antibody may be administered intravenously.

In one aspect, the present disclosure provides a dosing regimen for administering an anti-CD3 x anti-CD20 bispecific antibody to a subject to treat follicular lymphoma, comprising: (a) administering an initial dose of 0.7 mg of the bispecific antibody to the subject, wherein the initial dose is split into a first dose fraction comprising 0.2 mg of the bispecific antibody and a second dose fraction comprising 0.5 mg of the bispecific antibody, wherein the first dose fraction is administered to the subject followed by the second dose fraction over two days during week 1 of the dosing regimen; (b) administering a first intermediate dose of 4 mg of the bispecific antibody to the subject, wherein the first intermediate dose is split into two equal fractions (a first fraction and a second fraction), each comprising 2 mg of the bispecific antibody, wherein the two fractions of the first intermediate dose are administered over two days during week 2 of the dosing regimen; (c) administering a second intermediate dose of 20 mg of the bispecific antibody to the subject, wherein the second intermediate dose is split into two equal fractions (a first fraction and a second fraction), each comprising 10 mg of the bispecific antibody, wherein the two fractions of the second intermediate dose are administered over two days during week 3 of the dosing regimen; (d) administering a full dose of 40 mg or 80 mg of the bispecific antibody to the subject weekly during weeks 4 to 12 of the dosing regimen; and (e) administering a maintenance dose of 80 mg or 160 mg of the bispecific antibody to the subject in week 14 of the dosing regimen, wherein the bispecific antibody comprises a first antigen-binding region that binds human CD20 and a second antigen-binding region that binds human CD3, wherein the first antigen-binding region comprises three heavy chain complementarity determining regions, HCDR1, HCDR2 and HCDR3 comprising the amino acid sequences of SEQ ID NO: 7, 8 and 9, respectively, and three light chain complementarity determining regions LCDR1, LCDR2 and LCDR3 comprising the amino acid sequences of SEQ ID NO: 13, 14 and 15, respectively, and wherein the second antigen-binding region comprises three heavy chain complementarity determining regions, HCDR1, HCDR2 and HCDR3 comprising the amino acid sequences of SEQ ID NO: 10, 11 and 12, respectively, and three light chain complementarity determining regions LCDR1, LCDR2 and LCDR3 comprising the amino acid sequences of SEQ ID NO: 13, 14 and 15, respectively. In some embodiments, the initial dose (0.7 mg) may be split into other fractions, such as 0.1 mg and 0.6 mg, 0.15 mg and 0.55 mg, 0.25 mg and 0.45 mg, or 0.3 mg and 0.4 mg.

In one aspect, the present disclosure provides a dosing regimen for administering an anti-CD3 x anti-CD20 bispecific antibody to a subject to treat diffuse large B-cell lymphoma, comprising: (a) administering an initial dose of 0.7 mg of the bispecific antibody to the subject, wherein the initial dose is split into a first dose fraction comprising 0.2 mg of the bispecific antibody and a second dose fraction comprising 0.5 mg of the bispecific antibody, wherein the first dose fraction is administered to the subject followed by the second dose fraction over two days during week 1 of the dosing regimen; (b) administering a first intermediate dose of 4 mg of the bispecific antibody to the subject, wherein the first intermediate dose is split into two equal fractions (a first fraction and a second fraction), each comprising 2 mg of the bispecific antibody, wherein the two fractions of the first intermediate dose are administered over two days during week 2 of the dosing regimen; (c) administering a second intermediate dose of 20 mg of the bispecific antibody to the subject, wherein the second intermediate dose is split into two equal fractions (a first fraction and a second fraction), each comprising 10 mg of the bispecific antibody, wherein the two fractions of the second intermediate dose are administered over two days during week 3 of the dosing regimen; (d) administering a full dose of 80 mg or 160 mg of the bispecific antibody to the subject weekly during weeks 4 to 12 of the dosing regimen; and (e) administering a maintenance dose of 160 mg or 320 mg of the bispecific antibody to the subject in week 13 or week 14 of the dosing regimen, wherein the bispecific antibody comprises a first antigen-binding region that binds human CD20 and a second antigen-binding region that binds human CD3, wherein the first antigen-binding region comprises three heavy chain complementarity determining regions, HCDR1, HCDR2 and HCDR3 comprising the amino acid sequences of SEQ ID NO: 7, 8 and 9, respectively, and three light chain complementarity determining regions LCDR1, LCDR2 and LCDR3 comprising the amino acid sequences of SEQ ID NO: 13, 14 and 15, respectively, and wherein the second antigen-binding region comprises three heavy chain complementarity determining regions, HCDR1, HCDR2 and HCDR3 comprising the amino acid sequences of SEQ ID NO: 10, 11 and 12, respectively, and three light chain complementarity determining regions LCDR1, LCDR2 and LCDR3 comprising the amino acid sequences of SEQ ID NO: 13, 14 and 15, respectively. In some embodiments, the initial dose (0.7 mg) may be split into other fractions, such as 0.1 mg and 0.6 mg, 0.15 mg and 0.55 mg, 0.25 mg and 0.45 mg, or 0.3 mg and 0.4 mg.

In some embodiments, the full dose is administered to the subject as a single dose during weeks 4 to 12 of the dosing regimen.

In some embodiments, the maintenance dose is administered to the subject every two weeks beginning in week 13 of the dosing regimen. In some embodiments, the maintenance dose is administered to the subject every two weeks beginning in week 14 of the dosing regimen.

In some embodiments, the full dose is 40 mg. In some embodiments, the full dose is 80 mg. In some embodiments, the full dose is 160 mg. In some embodiments, the maintenance dose is 80 mg. In some embodiments, wherein the maintenance dose is 160 mg. In some embodiments, wherein the maintenance dose is 320 mg.

In some embodiments, the second dose fraction of the initial dose is administered to the subject from 18 to 96 hours after the first dose fraction of the initial dose. In some embodiments, the two fractions of the first intermediate dose are administered to the subject from 18 to 96 hours apart. In some embodiments, the two fractions of the second intermediate dose are administered to the subject from 18 to 96 hours apart.

In some embodiments, the dosing regimen further comprises administering a combination of cyclophosphamide, doxorubicin, vincristine, and prednisone (CHOP). In some embodiments, the dosing regimen further comprises administering a combination of cyclophosphamide, vincristine, and prednisone (CVP). In some cases, the CHOP/CVP is administered during a week preceding week 1 of the dosing regimen, and again during weeks 3, 6, 9. 12 and 15 of the dosing regimen. In some cases, the cyclophosphamide is administered at a dose of 750 mg/m², the doxorubicin is administered at a dose of 50 mg/m², the vincristine is administered at a dose of 1.4 mg/m², and the prednisone is administered at a dose of 100 mg, wherein the cyclophosphamide, doxorubicin and vincristine are administered once in the week preceding week 1 of the dosing regimen, and once in each of weeks 3, 6, 9, 12, 15 of the dosing regimen, and the prednisone is administered for five consecutive days in the week preceding week 1 of the dosing regimen, and for five consecutive days in each of weeks 3, 6, 9, 12 and 15 of the dosing regimen.

In some embodiments, the subject has relapsed or refractory disease. In some cases, the subject is refractory to an anti-CD20 antibody in any line of prior therapy. In some cases, the subject is double refractory to an alkylator and an anti-CD20 antibody.

In some embodiments, the subject has not been previously treated with a systemic anti-lymphoma therapy (i.e., the subject is previously untreated).

In some embodiments, the subject received a prior autologous stem cell transplant.

In some embodiments, the bispecific antibody is administered to the subject intravenously.

In some embodiments, the first antigen-binding region of the bispecific antibody comprises a heavy chain variable region (HCVR) comprising the amino acid sequence of SEQ ID NO: 4 and a light chain variable region (LCVR) comprising the amino acid sequence of SEQ ID NO: 6, and the second antigen-binding region of the bispecific antibody comprises a HCVR comprising the amino acid sequence of SEQ ID NO: 5 and a LCVR comprising the amino acid sequence of SEQ ID NO: 6.

In some embodiments, the bispecific antibody comprises a human IgG heavy chain constant region. In some cases, the human IgG heavy chain constant region is of isotype IgG1. In some cases, the human IgG heavy chain constant region is of isotype IgG4.

In some embodiments, the bispecific antibody comprises a first heavy chain constant region comprising the amino acid sequence of SEQ ID NO: 16 and a second heavy chain constant region comprising the amino acid sequence of SEQ ID NO: 17.

In some embodiments, the bispecific antibody comprises a first heavy chain constant region comprising the amino acid sequence of SEQ ID NO: 18 and a second heavy chain constant region comprising the amino acid sequence of SEQ ID NO: 19.

In some embodiments, the bispecific antibody comprises a first heavy chain comprising amino acid residues 1-452 of SEQ ID NO: 1 paired with a common light chain comprising the amino acid sequence of SEQ ID NO: 3, and a second heavy chain comprising amino acid residues 1-448 of SEQ ID NO: 2 paired with a common light chain comprising the amino acid sequence of SEQ ID NO: 3.

In some embodiments, the bispecific antibody comprises a first heavy chain comprising the amino acid sequence of SEQ ID NO: 1 paired with a common light chain comprising the amino acid sequence of SEQ ID NO: 3, and a second heavy chain comprising the amino acid sequence of SEQ ID NO: 2 paired with a common light chain comprising the amino acid sequence of SEQ ID NO: 3.

In any of the various embodiments discussed above or herein, the dosing regimen further comprises administering a dose of steroid to the subject: from 12 to 24 hours prior to the administration of the first dose fraction of the initial dose; from 12 to 24 hours prior to the administration of the first fraction of the first intermediate dose; and from 12 to 24 hours prior to the administration of the first fraction of the second intermediate dose.

In some embodiments, if the first dose fraction of the initial dose and the second dose fraction of the initial dose are not administered to the subject on consecutive days, then the dosing regimen further comprises administering a dose of steroid to the subject from 12 to 24 hours prior to the administration of the second dose fraction of the initial dose of the bispecific antibody. In some embodiments, if the first fraction of the first intermediate dose and the second fraction of the first intermediate dose are not administered to the subject on consecutive days, then the dosing regimen further comprises administering a dose of steroid to the subject from 12 to 24 hours prior to the administration of the second fraction of the first intermediate dose of the bispecific antibody. In some embodiments, if the first fraction of the second intermediate dose and the second fraction of the second intermediate dose are not administered to the subject on consecutive days, then the dosing regimen further comprises administering a dose of steroid to the subject from 12 to 24 hours prior to the administration of the second fraction of the second intermediate dose of the bispecific antibody.

In some embodiments, the dosing regimen further comprises: administering a dose of steroid to the subject: from 1 to 3 hours prior to the administration of the first dose fraction of the initial dose; from 1 to 3 hours prior to the administration of the second dose fraction of the initial dose; and from 1 to 3 hours prior to the administration of each fraction of the first intermediate dose and the second intermediate dose, and administering a dose of antihistamine to the subject: from 30 to 60 minutes prior to the administration of the first dose fraction of the initial dose; from 30 to 60 minutes prior to the administration of the second dose fraction of the initial dose; and from 30 to 60 minutes prior to the administration of each fraction of the first intermediate dose and the second intermediate dose.

In some embodiments, the dosing regimen further comprises administering a dose of acetaminophen to the subject: from 30 to 60 minutes prior to the administration of the first dose fraction of the initial dose; from 30 to 60 minutes prior to the administration of the second dose fraction of the initial dose; and from 30 to 60 minutes prior to the administration of each fraction of the first intermediate dose and the second intermediate dose.

In some embodiments, the dosing regimen further comprises administering a dose of steroid to the subject: from 20 to 28 hours after the end of administration of the second dose fraction of the initial dose; from 20 to 28 hours after the end of administration of the second fraction of the first intermediate dose; and from 20 to 28 hours after the end of administration of the second fraction of the second intermediate dose.

In some embodiments, the dosing regimen further comprises: administering a dose of steroid to the subject from 1 to 3 hours prior to the administration of the full dose during week 4 of the dosing regimen, and administering a dose of antihistamine to the subject from 30 to 60 minutes prior to the administration of the full dose during week 4 of the dosing regimen.

In some embodiments, the dosing regimen further comprises administering a dose of acetaminophen to the subject from 30 to 60 minutes prior to the administration of the full dose during week 4 of the dosing regimen.

In some embodiments, the dosing regimen further comprises administering a dose of steroid to the subject from 20 to 28 hours after the end of the administration of the full dose during week 4 of the dosing regimen.

In some embodiments, if the subject experiences a grade 3 event of cytokine release syndrome when administered the initial dose, the first intermediate dose, or the second intermediate dose, then the full dose of the bispecific antibody administered to the subject during week 4 is split into two equal fractions (a first fraction and a second fraction) and the two fractions of the full dose are administered over two days during week 4 of the dosing regimen, and wherein the dosing regimen further comprises administering a dose of steroid to the subject from 12 to 24 hours prior to the administration of the first fraction of the full dose. In some cases, if the first fraction of the full dose and the second fraction of the full dose are not administered to the subject on consecutive days, then the dosing regimen further comprises administering a dose of steroid to the subject from 12 to 24 hours prior to the administration of the second fraction of the full dose of the bispecific antibody. In some cases, the dosing regimen further comprises: administering a dose of steroid to the subject from 1 to 3 hours prior to the administration of the first fraction of the full dose, and administering a dose of antihistamine to the subject from 30 to 60 minutes prior to the administration of the first fraction of the full dose. In some cases, the dosing regimen further comprises administering a dose of acetaminophen to the subject from 30 to 60 minutes prior to the administration of the first fraction of the full dose. In some cases, the dosing regimen further comprises: administering a dose of steroid to the subject from 1 to 3 hours prior to the administration of the full dose during week 5 of the dosing regimen, and administering a dose of antihistamine to the subject from 30 to 60 minutes prior to the administration of the full dose during week 5 of the dosing regimen. In some cases, the dosing regimen further comprises administering a dose of acetaminophen to the subject from 30 to 60 minutes prior to the administration of the full dose during week 5 of the dosing regimen. In some cases, the dosing regimen further comprises administering a dose of steroid to the subject from 20 to 28 hours after the end of the administration of the full dose during week 5 of the dosing regimen.

In some embodiments, administering a dose of steroid, administering a dose of antihistamine, or administering a dose of acetaminophen comprises instructing the subject to ingest the dose of steroid, the dose of antihistamine, or the dose of acetaminophen, respectively.

In some embodiments, administering a dose of steroid, or administering a dose of antihistamine comprises intravenously administering the dose of steroid or the dose of antihistamine.

In some embodiments, the steroid is dexamethasone. In some cases, the dose of steroid is 20 mg.

In some embodiments, the antihistamine is diphenhydramine. In some cases, the dose of antihistamine is 25 mg.

In some embodiments, the dose of acetaminophen is 650 mg.

In any of the various embodiments discussed above or herein, the dosing regimen further comprises administering an anti-IL-6 receptor antibody. In some cases, the anti-IL-6 receptor antibody is tocilizumab or sarilumab.

In one aspect, the present disclosure provides a method of treating a B-cell cancer in a subject, comprising: selecting a subject diagnosed with a B-cell cancer; and administering the bispecific antibody to the subject according to the dosing regimen as discussed above or herein.

In various embodiments of the method of treatment: (a) the subject has been diagnosed with follicular lymphoma; (b) the subject has been diagnosed with follicular lymphoma of grade 1-3a; (c) the subject has been diagnosed with relapsed or refractory follicular lymphoma after at least 2 prior lines of systemic therapy; (d) the subject has been diagnosed with follicular lymphoma and has not previously been treated with a systemic anti-lymphoma therapy; (e) the subject has been diagnosed with follicular lymphoma, and the full dose is 80 mg; and/or (f) the subject has been diagnosed with follicular lymphoma, and the maintenance dose is 160 mg or 320 mg.

In various embodiments of the method of treatment: (a) the subject has been diagnosed with diffuse large B-cell lymphoma (DLBCL); (b) the subject has been diagnosed with DLBCL, and wherein the DLBCL is de novo or is transformed from a lower grade neoplasm; (c) the subject has been diagnosed with DLBCL and is refractory to at least 2 prior lines of systemic therapy; (d) the subject has been diagnosed with relapsed or refractory DLBCL after at least 2 prior lines of systemic therapy including CAR-T therapy; (e) the subject has been diagnosed with DLBCL and has not previously been treated with a systemic anti-lymphoma therapy; (f) the subject has been diagnosed with DLBCL, and the full dose is 160 mg; and/or (g) the subject has been diagnosed with DLBCL, and the maintenance dose is 320 mg.

The present disclosure also contemplates the use of an anti-CD20 x anti-CD3 antibody (e.g., odronextamab) for use in a method as discussed above or herein, as well as use of an anti-CD20 x anti-CD3 antibody (e.g., odronextamab) in the manufacture of a medicament for treating a B-cell cancer in a subject (e.g., FL or DLBCL) according to a method as discussed above or herein.

In one aspect, the present disclosure provides a pharmaceutical kit comprising (i) a container containing the bispecific antibody, and (ii) a label including instructions to administer the bispecific antibody according to the dosing regimen as discussed above or herein.

In some embodiments, the label further includes instructions to administer the steroid and antihistamine according to the dosing regimen as discussed above or herein.

In various embodiments, any of the features or components of any embodiments discussed above or herein may be combined, and such combinations are encompassed within the scope of the present disclosure. Any specific value discussed above or herein may be combined with another related value discussed above or herein to recite a range with the values representing the upper and lower ends of the range, and such ranges are encompassed within the scope of the present disclosure. A therapeutic protein for use in any of the methods discussed herein, or use of a therapeutic protein in the manufacture of a medicament for use in any of the methods discussed herein are also encompassed within the scope of this disclosure.

Other embodiments will become apparent from a review of the ensuing detailed description.

### BRIEF DESCRIPTION OF THE DRAWINGS

FIG. 1 illustrates odronextamab dosing regimens for treatment of patients with follicular lymphoma in accordance with an embodiment of the invention. *20 mg IV dexamethasone 1 to 3 hours prior to each split or initial single infusion; ^{†}10 mg dexamethasone orally 12 to 24 hours prior to the first split infusion. On each day of split or single infusion: dexamethasone 20 mg IV 1 to 3 hours before infusion; diphenhydramine 25 mg IV or orally and acetaminophen 650 mg orally 30 to 60 minutes before infusion. CRS, cytokine release syndrome; D, day; IV, intravenous; Q2W, every 2 weeks.

FIG. 2 shows that the majority of relapsed/refractory follicular lymphoma patients that were efficacy evaluable (N=121) had substantial tumor shrinkage following odronextamab monotherapy. The plot shows the best change (%) from baseline in tumor SPD (sum of the products of diameters). PD, progressive disease; CR/PR, complete response, partial response; NE, not estimable; SD, stable disease.

FIG. 3 shows the antitumor efficacy of odronextamab monotherapy in efficacy evaluable relapsed/refractory follicular lymphoma patients (N=121) and subgroups. ASCT, autologous stem cell transplant; CR, complete response; FLIPI, Follicular Lymphoma International Prognostic Index; ORR, objective response rate; POD24, progression of disease within 24 months of starting first-line therapy.

FIG. 4 shows the durability of responses and complete responses in relapsed/refractory follicular lymphoma patients that were treated with odronextamab monotherapy. CI, confidence interval; DOCR, duration of complete response; DOR, duration of response; NE, not estimable.

FIG. 5 shows progression-free survival and overall survival of relapsed/refractory follicular lymphoma patients that were treated with odronextamab monotherapy. CI, confidence interval; NE, not estimable; OS, overall survival; PFS, progression-free survival.

FIG. 6 illustrates odronextamab dosing regimens for treatment of patients with diffuse large B-cell lymphoma in accordance with an embodiment of the invention. *20 mg IV dexamethasone 1 to 3 hours prior to each split or initial single infusion. ^{†}10 mg dexamethasone orally 12 to 24 hours prior to the first split infusion. On each day of split or single infusion: dexamethasone 20 mg IV 1 to 3 hours before infusion; diphenhydramine 25 mg IV or orally and acetaminophen 650 mg orally 30 to 60 minutes before infusion. CRS, cytokine release syndrome; D, day; IV, intravenous; Q2W, every 2 weeks.

FIG. 7 shows the antitumor efficacy of odronextamab monotherapy in efficacy evaluable DLBCL patients (N=130) and subgroups. ASCT, autologous stem cell transplant; CI, confidence interval; CR, complete response; DLBCL, diffuse large B-cell lymphoma; ORR, objective response rate.

FIG. 8 shows the durability of responses and complete responses in relapsed/refractory DLBCL patients that were treated with odronextamab monotherapy. CI, confidence interval; DOOR, duration of complete response; DOR, duration of response; NE, not evaluable.

FIG. 9 shows progression-free survival of relapsed/refractory DLBCL patients that were treated with odronextamab monotherapy. CI, confidence interval; NE, not evaluable; PFS, progression-free survival.

FIG. 10 illustrates a dosing scheme for odronextamab plus chemotherapy, by cycle. The DLT observation period is defined as the first 35 days starting from C1D8 (first odronextamab administration) up to 2 full doses of odronextamab, whichever is later.

FIG. 11 illustrates a dosing scheme for rituximab plus chemotherapy, by cycle, used as a comparator.

### DETAILED DESCRIPTION

Before the present invention is described, it is to be understood that this invention is not limited to particular methods and experimental conditions described, as such methods and conditions may vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention belongs. The term “about,” when used in reference to a particular recited numerical value, means that the value may vary from the recited value by no more than 1%. For example, the expression “about 100” includes 99 and 101 and all values in between (e.g., 99.1, 99.2, 99.3, 99.4, etc.).

Although any methods and materials similar or equivalent to those described herein can be used in the practice or testing of the present invention, the preferred methods and materials are now described. All patents, applications and non-patent publications mentioned in this specification are incorporated herein by reference in their entireties.

### Definitions

All references to proteins, polypeptides and protein fragments herein are intended to refer to the human version of the respective protein, polypeptide or protein fragment unless explicitly specified as being from a non-human species. Thus, the expressions “CD3” and “CD20” mean human CD3 and human CD20, respectively, unless specified as being from a non-human species.

The expression “CD3,” refers to an antigen which is expressed on T cells as part of the multimolecular T cell receptor (TCR) and which consists of a homodimer or heterodimer formed from the association of two of four receptor chains: CD3-epsilon, CD3-delta, CD3-zeta, and CD3-gamma.

“An antigen-binding domain that binds CD3,” “an antigen-binding region that binds CD3,” “an antibody that binds CD3” or an “anti-CD3 antibody” includes antibodies and antigen-binding fragments thereof that specifically recognize a single CD3 subunit (e.g., epsilon, delta, gamma or zeta), as well as antibodies and antigen-binding fragments thereof that specifically recognize a dimeric complex of two CD3 subunits (e.g., gamma/epsilon, delta/epsilon, and zeta/zeta CD3 dimers). The antibodies and antigen-binding fragments of the present invention may bind soluble CD3 and/or cell surface expressed CD3. Soluble CD3 includes natural CD3 proteins as well as recombinant CD3 protein variants such as, e.g., monomeric and dimeric CD3 constructs, that lack a transmembrane domain or are otherwise unassociated with a cell membrane.

The expression “CD20,” refers to a non-glycosylated phosphoprotein expressed on the cell membranes of mature B cells. CD20 is considered a B cell tumor-associated antigen because it is expressed by more than 95% of B-cell non-Hodgkin lymphomas (NHLs) and other B-cell malignancies, but it is absent on precursor B-cells, dendritic cells and plasma cells.

“An antigen-binding domain that binds CD20,” “an antigen-binding region that binds CD20,” “an antibody that binds CD20” or an “anti-CD20 antibody” includes antibodies and antigen-binding fragments thereof that specifically recognize CD20.

“Cytokine release syndrome” (CRS) refers to a disorder characterized by fever, tachypnea, headache, tachycardia, hypotension, rash, and/or hypoxia, and is defined as an event that occurs 6 or more hours from the start of the infusion of the bispecific antibody or more than 2 hours after completion of the infusion of the bispecific antibody (whichever is later). Grades of CRS are defined in Tables 5, 6 and 11 of the Examples.

An “infusion related reaction” (IRR) refers to an adverse event that occurs less than 6 hours from the start of the infusion of the bispecific antibody and is associated with typical signs and symptoms including, but not limited to, flushing, tachycardia, hypotension, dyspnea, bronchospasm, back pain, fever, urticaria, edema, nausea, and rashes. As used herein, signs and symptoms of IRR that occur 6 or more hours from the start of the infusion are identified as cytokine release syndrome (CRS).

Non-Hodgkin lymphomas can be divided into two major prognostic groups: indolent (low-grade; slowly growing) lymphomas, and aggressive (high-grade; quickly growing) lymphomas. An “aggressive lymphoma” is a lymphoma characterized by one of the following subtypes based on the World Health Organization classification: a diffuse large B-cell lymphoma (DLBCL) not otherwise specified (NOS) by WHO classification; germinal center B-cell type; activated B-cell type; primary mediastinal (thymic) large B-cell lymphoma; T-cell/histiocyte-rich large B-cell lymphoma; Epstein-Barr virus (EBV)+ DLBCL, NOS; high-grade B-cell lymphoma, with MYC and BCL2 and/or BCL6 rearrangements; high-grade B-cell lymphoma, NOS; B-cell lymphoma, unclassifiable, with features intermediate between DLBCL and classical Hodgkin lymphoma; and follicular lymphoma, grade 3b. Follicular lymphoma, grade 1-3a, is the most common form of indolent lymphoma.

The term “antibody”, means any antigen-binding molecule or molecular complex comprising at least one complementarity determining region (CDR) that specifically binds to or interacts with a particular antigen (e.g., CD20 or CD3). The term “antibody” includes immunoglobulin molecules comprising four polypeptide chains, two heavy (H) chains and two light (L) chains inter-connected by disulfide bonds, as well as multimers thereof (e.g., IgM). The term “antibody” also includes immunoglobulin molecules consisting of four polypeptide chains, two heavy (H) chains and two light (L) chains inter-connected by disulfide bonds. Each heavy chain comprises a heavy chain variable region (abbreviated herein as HCVR or V_{H}) and a heavy chain constant region. The heavy chain constant region comprises three domains, C_{H}1, C_{H}2 and C_{H}3. Each light chain comprises a light chain variable region (abbreviated herein as LCVR or V_{L}) and a light chain constant region. The light chain constant region comprises one domain (C_{L}1). The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. In different embodiments of the invention, the FRs of the antibody (or antigen-binding portion thereof) may be identical to the human germline sequences, or may be naturally or artificially modified. An amino acid consensus sequence may be defined based on a side-by-side analysis of two or more CDRs. The term “antibody” includes a “bispecific antibody” unless otherwise noted.

The term “antibody”, also includes antigen-binding fragments of full antibody molecules. The terms “antigen-binding portion” of an antibody, “antigen-binding fragment” of an antibody, and the like, include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

The expression “bispecific antigen-binding molecule” refers to a protein, polypeptide or molecular complex comprising at least a first antigen-binding domain and a second antigen-binding domain. Each antigen-binding domain within the bispecific antigen-binding molecule comprises at least one CDR that alone, or in combination with one or more additional CDRs and/or FRs, specifically binds to a particular antigen. Bispecific antigen-binding molecules include bispecific antibodies.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains, are also encompassed within the expression “antigen-binding fragment”.

An antigen-binding fragment of an antibody will typically comprise at least one variable domain. The variable domain may be of any size or amino acid composition and will generally comprise at least one CDR which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}—V_{H}, V_{H}-V_{L} or V_{L}-V_{L} dimers. Alternatively, the antigen-binding fragment of an antibody may contain a monomeric V_{H} or V_{L} domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody of the present invention include: (i) V_{H}-C_{H}1; (ii) V_{H}-C_{H}2; (iii) V_{H}-C_{H}3; (iv) V_{H}-C_{H}1-C_{H}2; (V) V_{H}-C_{H}1-C_{H}2-C_{H}3; (vi) V_{H}-C_{H}2-C_{H}3; V_{H}-C_{L}; V_{L}-C_{H}1; (ix) V_{L}-C_{H}2; (X) V_{L}-C_{H}3; (Xi) V_{L}-C_{H}1-C_{H}2; (XII) V_{L}-C_{H}1-C_{H}2-C_{H}3; (Xiii) V_{L}-C_{H}2-C_{H}3; and (xiv) V_{L}-C_{L}. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids which result in a flexible or semi-flexible linkage between adjacent variable and/or constant domains in a single polypeptide molecule. Moreover, an antigen-binding fragment of an antibody of the present invention may comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)).

As with full antibody molecules, antigen-binding fragments may be monospecific or multispecific (e.g., bispecific). A multispecific antigen-binding fragment of an antibody will typically comprise at least two different variable domains, wherein each variable domain is capable of specifically binding to a separate antigen or to a different epitope on the same antigen. Any multispecific antibody format, including the exemplary bispecific antibody formats disclosed herein, may be adapted for use in the context of an antigen-binding fragment of an antibody of the present invention using routine techniques available in the art.

The antibodies of the present disclosure may function through complement-dependent cytotoxicity (CDC) or antibody-dependent cell-mediated cytotoxicity (ADCC). “Complement-dependent cytotoxicity” (CDC) refers to lysis of antigen-expressing cells by an antibody of the invention in the presence of complement. “Antibody-dependent cell-mediated cytotoxicity” (ADCC) refers to a cell-mediated reaction in which nonspecific cytotoxic cells that express Fc receptors (FcRs) (e.g., Natural Killer (NK) cells, neutrophils, and macrophages) recognize bound antibody on a target cell and thereby lead to lysis of the target cell. CDC and ADCC can be measured using assays that are well known and available in the art. (See, e.g., U.S. Pat. Nos. 5,500,362 and 5,821,337, and Clynes et al. (1998) Proc. Natl. Acad. Sci. (USA) 95:652-656). The constant region of an antibody is important in the ability of an antibody to fix complement and mediate cell-dependent cytotoxicity. Thus, the isotype of an antibody may be selected on the basis of whether it is desirable for the antibody to mediate cytotoxicity. Antibodies of the present disclosure may include a human IgG heavy chain. In various embodiments, the heavy chain constant region may be of IgG1, IgG2, IgG3 or IgG4 isotype. In some cases, the heavy chain constant region is of isotype IgG1. In some cases, the heavy chain constant region is of isotype IgG4.

In certain embodiments, the antibodies or bispecific antibodies are human antibodies. The term “human antibody” is intended to include antibodies having variable and constant regions derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody” is not intended to include antibodies in which CDR sequences derived from the germline of another mammalian species, such as a mouse, have been grafted onto human framework sequences.

The antibodies of the invention may, in some embodiments, be recombinant human antibodies. The term “recombinant human antibody” is intended to include all human antibodies that are prepared, expressed, created or isolated by recombinant means, such as antibodies expressed using a recombinant expression vector transfected into a host cell (described further below), antibodies isolated from a recombinant, combinatorial human antibody library (described further below), antibodies isolated from an animal (e.g., a mouse) that is transgenic for human immunoglobulin genes (see e.g., Taylor et al. (1992) Nucl. Acids Res. 20:6287-6295) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

Human antibodies can exist in two forms that are associated with hinge heterogeneity. In one form, an immunoglobulin molecule comprises a stable four chain construct of approximately 150-160 kDa in which the dimers are held together by an interchain heavy chain disulfide bond. In a second form, the dimers are not linked via inter-chain disulfide bonds and a molecule of about 75-80 kDa is formed composed of a covalently coupled light and heavy chain (half-antibody). These forms have been extremely difficult to separate, even after affinity purification.

The frequency of appearance of the second form in various intact IgG isotypes is due to, but not limited to, structural differences associated with the hinge region isotype of the antibody. A single amino acid substitution in the hinge region of the human IgG4 hinge can significantly reduce the appearance of the second form (Angal et al. (1993) Molecular Immunology 30:105) to levels typically observed using a human IgG1 hinge. The instant invention encompasses antibodies having one or more mutations in the hinge, C_{H}2 or C_{H}3 region which may be desirable, for example, in production, to improve the yield of the desired antibody form.

The antibodies may be isolated antibodies. An “isolated antibody” means an antibody that has been identified and separated and/or recovered from at least one component of its natural environment. For example, an antibody that has been separated or removed from at least one component of an organism, or from a tissue or cell in which the antibody naturally exists or is naturally produced, is an “isolated antibody” for purposes of the present invention. An isolated antibody also includes an antibody in situ within a recombinant cell. Isolated antibodies are antibodies that have been subjected to at least one purification or isolation step. According to certain embodiments, an isolated antibody may be substantially free of other cellular material and/or chemicals.

The term “epitope” refers to an antigenic determinant that interacts with a specific antigen binding site in the variable region of an antibody molecule known as a paratope. A single antigen may have more than one epitope. Thus, different antibodies may bind to different areas on an antigen and may have different biological effects. Epitopes may be either conformational or linear. A conformational epitope is produced by spatially juxtaposed amino acids from different segments of the linear polypeptide chain. A linear epitope is one produced by adjacent amino acid residues in a polypeptide chain. In certain circumstance, an epitope may include moieties of saccharides, phosphoryl groups, or sulfonyl groups on the antigen.

The term “substantial identity” or “substantially identical,” when referring to a nucleic acid or fragment thereof, indicates that, when optimally aligned with appropriate nucleotide insertions or deletions with another nucleic acid (or its complementary strand), there is nucleotide sequence identity in at least about 95%, and more preferably at least about 96%, 97%, 98% or 99% of the nucleotide bases, as measured by any well-known algorithm of sequence identity, such as FASTA, BLAST or Gap, as discussed below. A nucleic acid molecule having substantial identity to a reference nucleic acid molecule may, in certain instances, encode a polypeptide having the same or substantially similar amino acid sequence as the polypeptide encoded by the reference nucleic acid molecule.

As applied to polypeptides, the term “substantial similarity” or “substantially similar” means that two peptide sequences, when optimally aligned, such as by the programs GAP or BESTFIT using default gap weights, share at least 95% sequence identity, even more preferably at least 98% or 99% sequence identity. Preferably, residue positions which are not identical differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of similarity may be adjusted upwards to correct for the conservative nature of the substitution. Means for making this adjustment are well-known to those of skill in the art. See, e.g., Pearson (1994) Methods Mol. Biol. 24: 307-331, herein incorporated by reference.

Examples of groups of amino acids that have side chains with similar chemical properties include (1) aliphatic side chains: glycine, alanine, valine, leucine and isoleucine; (2) aliphatic-hydroxyl side chains: serine and threonine; (3) amide-containing side chains: asparagine and glutamine; (4) aromatic side chains: phenylalanine, tyrosine, and tryptophan; (5) basic side chains: lysine, arginine, and histidine; (6) acidic side chains: aspartate and glutamate, and (7) sulfur-containing side chains are cysteine and methionine. Preferred conservative amino acids substitution groups are: valine-leucine-isoleucine, phenylalanine-tyrosine, lysine-arginine, alanine-valine, glutamate-aspartate, and asparagine-glutamine. Alternatively, a conservative replacement is any change having a positive value in the PAM250 log-likelihood matrix disclosed in Gonnet et al. (1992) Science 256: 1443-1445, herein incorporated by reference. A “moderately conservative” replacement is any change having a nonnegative value in the PAM250 log-likelihood matrix.

Sequence similarity for polypeptides, which is also referred to as sequence identity, is typically measured using sequence analysis software. Protein analysis software matches similar sequences using measures of similarity assigned to various substitutions, deletions and other modifications, including conservative amino acid substitutions. For instance, GCG software contains programs such as Gap and Bestfit which can be used with default parameters to determine sequence homology or sequence identity between closely related polypeptides, such as homologous polypeptides from different species of organisms or between a wild type protein and a mutein thereof. See, e.g., GCG Version 6.1. Polypeptide sequences also can be compared using FASTA using default or recommended parameters, a program in GCG Version 6.1. FASTA (e.g., FASTA2 and FASTA3) provides alignments and percent sequence identity of the regions of the best overlap between the query and search sequences (Pearson (2000) supra). Another preferred algorithm when comparing a sequence of the invention to a database containing a large number of sequences from different organisms is the computer program BLAST, especially BLASTP or TBLASTN, using default parameters. See, e.g., Altschul et al. (1990) J. Mol. Biol. 215:403-410 and Altschul et al. (1997) Nucleic Acids Res. 25:3389-402, each herein incorporated by reference.

### Dosing Regimens

Dosing regimens to mitigate the prevalence or severity, or both, of cytokine release syndrome (CRS) or infusion-related reaction (IRR) by administration of a bispecific antibody to a patient to treat B-cell malignancies are provided in the present disclosure. According to certain embodiments of the present invention, these dosing regimens include administration of multiple doses of the bispecific antibody over a defined time course. The dosing regimens include sequential administration to a subject of multiple defined doses of a bispecific antibody, along with administration of premedications (e.g., steroids and antihistamines) to minimize or eliminate the risk of adverse events associated with administration of a T-cell engaging bispecific antibody. “Sequential administration” means that each dose of the bispecific antibody is administered to the subject at a different point in time, e.g., on different days separated by a predetermined interval (e.g., hours, days, weeks or months). The present invention includes dosing regimens that comprise sequential administration of split doses of the bispecific antibody followed by single dose administration of the bispecific antibody. The present administration regimens allow for higher doses of the therapeutic protein that are desirable for enhancing therapeutic efficacy, but without the deleterious effects associated with CRS or IRR. Without intending to be bound by any particular theory, the present administration regimens provide for priming of the immune response to administration of the bispecific antibodies to minimize the incidence and severity of CRS and IRR during initial phases of the treatment regimen, which then permits administration of higher doses of the bispecific antibodies during subsequent phases of the treatment regimen without significant adverse events associated with CRS or IRR.

A dosing regimen in accordance with the present invention includes administering an anti-CD3 x anti-CD20 bispecific antibody to a subject to treat a B-cell malignancy, wherein the dosing regimen comprises: (a) administering an initial dose of 0.7 mg of the bispecific antibody to the subject, wherein the initial dose is split into a first dose fraction comprising 0.2 mg of the bispecific antibody and a second dose fraction comprising 0.5 mg of the bispecific antibody, wherein the first dose fraction is administered to the subject followed by the second dose fraction over two days during week 1 of the dosing regimen; (b) administering a first intermediate dose of 4 mg of the bispecific antibody to the subject, wherein the first intermediate dose is split into two equal fractions (a first fraction and a second fraction), each comprising 2 mg of the bispecific antibody, wherein the two fractions of the first intermediate dose are administered over two days during week 2 of the dosing regimen; (c) administering a second intermediate dose of 20 mg of the bispecific antibody to the subject, wherein the second intermediate dose is split into two equal fractions (a first fraction and a second fraction), each comprising 10 mg of the bispecific antibody, wherein the two fractions of the second intermediate dose are administered over two days during week 3 of the dosing regimen; (d) administering a full dose of the bispecific antibody to the subject weekly during weeks 4 to 12 of the dosing regimen; and (e) administering a maintenance dose of the bispecific antibody to the subject in week 14 of the dosing regimen. In some embodiments, the initial dose (0.7 mg) may be split into other fractions, such as 0.1 mg and 0.6 mg, 0.15 mg and 0.55 mg, 0.25 mg and 0.45 mg, or 0.3 mg and 0.4 mg.

In the absence of a grade 3 or higher event of CRS when the subject is administered the initial dose, the first intermediate dose, or the second intermediate dose, then the full dose is administered to the subject as a single dose during weeks 4 to 12 of the dosing regimen. If, on the other hand, the subject experiences a grade 3 or higher event of CRS when administered the initial dose, the first intermediate dose, or the second intermediate dose, then the full dose of the bispecific antibody administered to the subject during week 4 is split into two equal fractions and the two fractions of the full dose are administered over two days during week 4 of the dosing regimen, and thereafter the full dose is administered to the subject as a single dose during weeks 5 to 12 of the dosing regimen.

Because the weekly dosing of the full dose proceeds from week 4 through week 12 in the dosing regimen, the first maintenance dose, which is administered every other week (Q2W), will be administered beginning in week 14 of the dosing regimen. The Q2W dosing of the maintenance dose may continue indefinitely, or may be modified to administration of the maintenance dose every four weeks (Q4W) if, e.g., the subject has demonstrated a durable response for at least nine months after an initial determination of a complete response. Thus, in some embodiments, the maintenance dose is administered to the subject every four weeks beginning in a subsequent week of the dosing regimen, wherein the subsequent week is at least week 36 of the dosing regimen. In various embodiments, the subsequent week may be week 36, 37, 38, 39, 40, 41, 42, 43, 44, 45, 46, 47, 48, 49, 50, 51, 52, 53, 54, 55, 56, 57, 58, 59, or week 60, or a higher numbered week of the dosing regimen. This Q4W dosing of the maintenance dose may also continue indefinitely.

In certain embodiments, the weekly dosing of the full dose proceeds from week 4 through week 6 in the dosing regimen, the first maintenance dose, which is administered every other week (Q2W), will be administered beginning in week 8 of the dosing regimen. The Q2W dosing of the maintenance dose may continue for 2 cycles at least after which it may be modified to administration of the maintenance dose every eight weeks (Q8W). Thus, in some embodiments, the maintenance dose is administered to the subject every eight weeks beginning in a subsequent week of the dosing regimen, wherein the subsequent week is at least week 18 of the dosing regimen. In various embodiments, the subsequent week may be week 19, 20, 21, 22, 23, 24, 25, 26, 27, 28, 29, 30, 31, 32, 33, 34, 35, 36, or a higher numbered week of the dosing regimen. This Q8W dosing of the maintenance dose may also continue indefinitely.

In certain embodiments, the dosing regimen may comprise a combination therapy including a bispecific anti-CD20 x anti-CD3 antibody (e.g., odronextamab) and CHOP (a combination of cyclophosphamide, doxorubicin, vincristine, and prednisone). In certain embodiments, the dosing regimen may comprise a combination therapy including a bispecific anti-CD20 x anti-CD3 antibody (e.g., odronextamab) and CVP (a combination of cyclophosphamide, vincristine, and prednisone). In embodiments in which the bispecific antibody is combined with CHOP or CVP, the first CHOP/CVP administration may be given in the week preceding the first week of the dosing regimen (the first week is when the first dose of the bispecific antibody is administered), and first maintenance dose of the bispecific antibody may be administered in week 13 (rather than week 14) of the dosing regimen. In some embodiments, the CHOP/CVP is administered, in addition to the week preceding the first week (i.e., in week 0 of the dosing regimen), during weeks 3, 6, 9, 12 and 15 of the dosing regimen.

In some embodiments, the cyclophosphamide is administered at a dose of 750 mg/m², the doxorubicin is administered at a dose of 50 mg/m², the vincristine is administered at a dose of 1.4 mg/m² (but no more than 2 mg in any dose), and the prednisone is administered at a dose of 100 mg, wherein the cyclophosphamide, doxorubicin and vincristine are administered once in week 0 of the dosing regimen, and once in each of weeks 3, 6, 9, 12, 15 of the dosing regimen, and the prednisone is administered for five consecutive days in week 0 of the dosing regimen, and for five consecutive days in each of weeks 3, 6, 9, 12 and 15 of the dosing regimen.

As noted above, the split doses (i.e., the fractions of the initial, first intermediate, and second intermediate doses) are administered over two days during the relevant week of the dosing regimen. In this context, the two days refers to two calendar days (e.g., Monday and Tuesday, or May 10th and May 11th)). In some cases, the two days are consecutive days. In some cases, the two days are not consecutive days, but are no more than three days apart. For example, the first dose fraction of the initial dose may be administered on a Monday, and the second dose fraction of the initial dose may be administered on Wednesday or Thursday. In some cases, the second dose fraction of the initial dose is administered to the subject from 18 to 96 hours after the first dose fraction of the initial dose. In some cases, the second dose fraction of the initial dose is administered to the subject from 18 to 72 hours after the first dose fraction of the initial dose. In some cases, the second dose fraction of the initial dose is administered to the subject from 24 to 48 hours after the first dose fraction of the initial dose. In some cases, the two fractions of the first intermediate dose are administered to the subject from 18 to 96 hours apart. In some cases, the two fractions of the first intermediate dose are administered to the subject from 18 to 72 hours apart. In some cases, the two fractions of the first intermediate dose are administered to the subject from 24 to 48 hours apart. In some cases, the two fractions of the second intermediate dose are administered to the subject from 18 to 96 hours apart. In some cases, the two fractions of the second intermediate dose are administered to the subject from 18 to 72 hours apart. In some cases, the two fractions of the second intermediate dose are administered to the subject from 24 to 48 hours apart. In instances in which the full dose is split into two fractions, the two fractions of the full dose are administered to the subject from 18 to 96 hours apart. In some cases, the two fractions of the full dose are administered to the subject from 18 to 72 hours apart. In some cases, the two fractions of the full dose are administered to the subject from 24 to 48 hours apart. In any of these embodiments, irrespective of the number of hours between administration of the two fractions (e.g., 18 hours), the two fractions are administered on different calendar days.

In any of the various dosing regimens, the B-cell malignancy for which the subject is undergoing treatment may be a B-cell non-Hodgkin lymphoma. In some cases, the B-cell malignancy is follicular lymphoma, diffuse large B-cell lymphoma, mantle cell lymphoma, or marginal zone lymphoma. In various embodiments, the subject is a human adult (18 years or older) diagnosed with the specific B-cell malignancy.

In various embodiments of the dosing regimens, the full dose of the bispecific antibody is from 40 mg to 320 mg, or 80 mg to 320 mg. In various embodiments of the dosing regimen, the maintenance dose of the bispecific antibody is from 80 mg to 320 mg, or 160 mg to 320 mg. The full dose and/or the maintenance dose of the bispecific antibody may vary depending on the specific type of B-cell malignancy being treated. For example, when the cancer is follicular lymphoma (e.g., grade 1-3a), the full dose is 80 mg and the maintenance dose is 160 mg. In another example, when the cancer is diffuse large B-cell lymphoma (e.g., DLBCL in which the subject failed prior CAR-T therapy), the full dose is 160 mg and the maintenance dose is 320 mg, or the full dose is 320 mg and the maintenance dose is 320 mg. In another example, when the cancer is mantle cell lymphoma (e.g., MCL in which the subject failed prior Bruton tyrosine kinase (BTK) inhibitor therapy), the full dose is 160 mg and the maintenance dose is 320 mg. In another example, when the cancer is marginal zone lymphoma, the full dose is 80 mg and the maintenance dose is 160 mg. In another example, when the cancer is a non-Hodgkin lymphoma other than follicular lymphoma, diffuse large B-cell lymphoma, mantle cell lymphoma, or marginal zone lymphoma, the full dose is 160 mg and the maintenance dose is 320 mg. In another example, when the cancer is an aggressive lymphoma, the full dose is 160 mg and the maintenance dose is 320 mg.

Although it is contemplated that a subject will receive the doses discussed herein in the dosing regimens in sequential weeks, i.e., where week 2 follows week 1, and week 3 follows week 2, etc, in terms of calendar weeks, it is possible in some instances that there may be a delay in the administration of a dose such that the next week will not immediately follow the preceding calendar week. For example, week 1 of the dosing regimen may correspond to calendar week 1, and week 2 of the dosing regimen may correspond to calendar week 3, or calendar week 4.

In various embodiments, the dosing regimen further comprises administering a dose of steroid to the subject: from 12 to 24 hours prior to the administration of the first dose fraction of the initial dose; from 12 to 24 hours prior to the administration of the first fraction of the first intermediate dose; and from 12 to 24 hours prior to the administration of the first fraction of the second intermediate dose. In those instances in which the first dose fraction of the initial dose and the second dose fraction of the initial dose are not administered to the subject on consecutive days (i.e., consecutive calendar days), then the dosing regimen further comprises administering a dose of steroid to the subject from 12 to 24 hours prior to the administration of the second dose fraction of the initial dose of the bispecific antibody. Similarly, if the first fraction of the first intermediate dose and the second fraction of the first intermediate dose are not administered to the subject on consecutive days, and/or the first fraction of the second intermediate dose and the second fraction of the second intermediate dose are not administered to the subject on consecutive days then the dosing regimen further comprises administering a dose of steroid to the subject from 12 to 24 hours prior to the administration of the second fraction of the first intermediate dose of the bispecific antibody and/or administering a dose of steroid to the subject from 12 to 24 hours prior to the administration of the second fraction of the first intermediate dose of the bispecific antibody, respectively.

In some embodiments of the dosing regimen, the subject has relapsed or refractory disease. In some embodiments of the dosing regimen, the subject is refractory to an anti-CD20 antibody in any line of prior therapy.

In some embodiments of the dosing regimen, the subject is previously untreated, i.e., has not been previously treated with any systemic anti-lymphoma therapy.

In some embodiments of the dosing regimen, the B-cell malignancy is follicular lymphoma. In some embodiments of the dosing regimen, the B-cell malignancy is diffuse large B-cell lymphoma.

In some embodiments of the dosing regimen, the subject is a human aged 18 years.

In various embodiments of the dosing regimen discussed above, the bispecific antibody is administered to the subject intravenously.

In other embodiments of the dosing regimen, the bispecific antibody is administered to the subject subcutaneously (SC).

An exemplary SC dosing regimen comprises: administering an initial dose of 1 mg or 2 mg of the bispecific antibody to the subject during week 1 of the dosing regimen; administering a first intermediate dose of 10 mg or 26 mg of the bispecific antibody to the subject during week 2 of the dosing regimen; administering a second intermediate dose of 50 mg or 100 mg of the bispecific antibody to the subject during week 3 of the dosing regimen; and administering a full dose of the bispecific antibody to the subject during week 4 and during a subsequent week of the dosing regimen, wherein the bispecific antibody comprises a first antigen-binding region that binds human CD20 and a second antigen-binding region that binds human CD3, wherein the first antigen-binding region comprises three heavy chain complementarity determining regions, HCDR1, HCDR2 and HCDR3 comprising the amino acid sequences of SEQ ID NO: 7, 8 and 9, respectively, and three light chain complementarity determining regions LCDR1, LCDR2 and LCDR3 comprising the amino acid sequences of SEQ ID NO: 13, 14 and 15, respectively, and wherein the second antigen-binding region comprises three heavy chain complementarity determining regions, HCDR1, HCDR2 and HCDR3 comprising the amino acid sequences of SEQ ID NO: 10, 11 and 12, respectively, and three light chain complementarity determining regions LCDR1, LCDR2 and LCDR3 comprising the amino acid sequences of SEQ ID NO: 13, 14 and 15, respectively.

In some embodiments of the SC dosing regimen, the initial dose is 2 mg. In some embodiments of the SC dosing regimen, the first intermediate dose is 26 mg. In some embodiments of the SC dosing regimen, the second intermediate dose is 100 mg. In some embodiments of the SC dosing regimen, the full dose is 200 mg, 400 mg or 600 mg. In some cases, the full dose is 400 mg. In some cases, the full dose is 600 mg.

In some embodiments of the SC dosing regimen, the full dose is administered to the subject once every three weeks. In some embodiments of the SC dosing regimen, the full dose is administered to the subject weekly. In some cases of the SC dosing regimen, the full dose is administered to the subject weekly for three weeks, and then the full dose is administered to the subject once every three weeks.

In some embodiments of the SC dosing regimen, the subject has relapsed or refractory disease. In some embodiments of the SC dosing regimen, the subject is refractory to an anti-CD20 antibody in any line of prior therapy.

In some embodiments of the SC dosing regimen, the subject is previously untreated, i.e., has not been previously treated with any systemic anti-lymphoma therapy.

In some embodiments of the SC dosing regimen, the B-cell malignancy is follicular lymphoma. In some embodiments of the SC dosing regimen, the B-cell malignancy is diffuse large B-cell lymphoma.

In some embodiments of the SC dosing regimen, the subject is a human aged 18 years.

In some embodiments, the dosing regimen is intended for administration of the bispecific antibody to a pediatric subject (e.g., less than 18 years of age).

An exemplary pediatric dosing regimen comprises: administering an initial dose of the bispecific antibody to the subject, wherein the initial dose is split into a first dose fraction of the bispecific antibody and a second dose fraction of the bispecific antibody, wherein the first dose fraction is administered to the subject followed by the second dose fraction over two days during week 1 of the dosing regimen; administering a first intermediate dose of the bispecific antibody to the subject, wherein the first intermediate dose is split into two equal fractions (a first fraction and a second fraction) of the bispecific antibody, wherein the two fractions of the first intermediate dose are administered over two days during week 2 of the dosing regimen; administering a second intermediate dose of the bispecific antibody to the subject, wherein the second intermediate dose is split into two equal fractions (a first fraction and a second fraction) of the bispecific antibody, wherein the two fractions of the second intermediate dose are administered over two days during week 3 of the dosing regimen; administering a full dose of the bispecific antibody to the subject weekly during weeks 4 to 12 of the dosing regimen; and administering a maintenance dose of the bispecific antibody to the subject in week 14 of the dosing regimen, wherein the initial dose, the first intermediate dose, the second intermediate dose, the full dose and the maintenance dose is dependent on the weight of subject: (i) if the subject has a weight of from 40 kg to less than 165 kg, the initial dose is 0.7 mg, the first dose fraction of the initial dose is 0.2 mg, the second dose fraction of the initial dose is 0.5 mg, the first intermediate dose is 4 mg, the two equal fractions of the first intermediate dose each comprise 2 mg, the second intermediate dose is 20 mg, the two equal fractions of the second intermediate dose each comprise 10 mg, the full dose is 160 mg, and the maintenance dose is 320 mg; (ii) if the subject has a weight of from 20 kg to less than 40 kg, the initial dose is 0.4 mg, the first dose fraction of the initial dose is 0.1 mg, the second dose fraction of the initial dose is 0.3 mg, the first intermediate dose is 2 mg, the two equal fractions of the first intermediate dose each comprise 1 mg, the second intermediate dose is 12 mg, the two equal fractions of the second intermediate dose each comprise 6 mg, the full dose is 90 mg, and the maintenance dose is 150 mg; (iii) if the subject has a weight of from 10 kg to less than 20 kg, the initial dose is 0.3 mg, the first dose fraction of the initial dose is 0.1 mg, the second dose fraction of the initial dose is 0.2 mg, the first intermediate dose is 1.6 mg, the two equal fractions of the first intermediate dose each comprise 0.8 mg, the second intermediate dose is 8 mg, the two equal fractions of the second intermediate dose each comprise 4 mg, the full dose is 60 mg, and the maintenance dose is 100 mg; or (iv) if the subject has a weight of from 6 kg to less than 10 kg, the initial dose is 0.24 mg, the first dose fraction of the initial dose is 0.07 mg, the second dose fraction of the initial dose is 0.17 mg, the first intermediate dose is 1.2 mg, the two equal fractions of the first intermediate dose each comprise 0.6 mg, the second intermediate dose is 6 mg, the two equal fractions of the second intermediate dose each comprise 3 mg, the full dose is 45 mg, and the maintenance dose is 75 mg; and wherein the bispecific antibody comprises a first antigen-binding region that binds human CD20 and a second antigen-binding region that binds human CD3, wherein the first antigen-binding region comprises three heavy chain complementarity determining regions, HCDR1, HCDR2 and HCDR3 comprising the amino acid sequences of SEQ ID NO: 7, 8 and 9, respectively, and three light chain complementarity determining regions LCDR1, LCDR2 and LCDR3 comprising the amino acid sequences of SEQ ID NO: 13, 14 and 15, respectively, and wherein the second antigen-binding region comprises three heavy chain complementarity determining regions, HCDR1, HCDR2 and HCDR3 comprising the amino acid sequences of SEQ ID NO: 10, 11 and 12, respectively, and three light chain complementarity determining regions LCDR1, LCDR2 and LCDR3 comprising the amino acid sequences of SEQ ID NO: 13, 14 and 15, respectively.

In some embodiments of the pediatric dosing regimen, the full dose is administered to the subject as a single dose during weeks 4 to 12 of the dosing regimen. In some embodiments of the pediatric dosing regimen, the maintenance dose is administered to the subject every two weeks beginning in week 14 of the dosing regimen.

In some embodiments of the pediatric dosing regimen, the second dose fraction of the initial dose is administered to the subject from 18 to 96 hours after the first dose fraction of the initial dose. In some embodiments of the pediatric dosing regimen, the two fractions of the first intermediate dose are administered to the subject from 18 to 96 hours apart. In some embodiments of the pediatric dosing regimen, the two fractions of the second intermediate dose are administered to the subject from 18 to 96 hours apart.

In some embodiments of the pediatric dosing regimen, the two days are consecutive days. In some embodiments of the pediatric dosing regimen, the two days are no more than three days apart.

In some embodiments of the pediatric dosing regimen, the B-cell malignancy is a B-cell non-Hodgkin lymphoma. In some embodiments of the pediatric dosing regimen, the B-cell malignancy is follicular lymphoma, diffuse large B-cell lymphoma, mantle cell lymphoma, or marginal zone lymphoma.

In any of the various embodiments of the pediatric dosing regimen, the bispecific antibody may be administered intravenously.

In various embodiments, the dosing regimen further comprises: administering a dose of steroid to the subject: from 1 to 3 hours prior to the administration of the first dose fraction of the initial dose; from 1 to 3 hours prior to the administration of the second dose fraction of the initial dose; and from 1 to 3 hours prior to the administration of each fraction of the first intermediate dose and the second intermediate dose, and administering a dose of antihistamine to the subject: from 30 to 60 minutes prior to the administration of the first dose fraction of the initial dose; from 30 to 60 minutes prior to the administration of the second dose fraction of the initial dose; and from 30 to 60 minutes prior to the administration of each fraction of the first intermediate dose and the second intermediate dose, and optionally administering a dose of acetaminophen to the subject: from 30 to 60 minutes prior to the administration of the first dose fraction of the initial dose; from 30 to 60 minutes prior to the administration of the second dose fraction of the initial dose; and from 30 to 60 minutes prior to the administration of each fraction of the first intermediate dose and the second intermediate dose.

In various embodiments, the dosing regimen further comprises administering a dose of steroid to the subject: from 20 to 28 hours after the end of administration of the second dose fraction of the initial dose; from 20 to 28 hours after the end of administration of the second fraction of the first intermediate dose; and from 20 to 28 hours after the end of administration of the second fraction of the second intermediate dose.

In various embodiments in which the subject does not experience a grade 3 or higher event of CRS when the subject is administered the initial dose, the first intermediate dose, or the second intermediate dose, and the full dose is administered to the subject as a single dose during week 4, the dosing regimen further comprises: administering a dose of steroid to the subject from 1 to 3 hours prior to the administration of the full dose during week 4 of the dosing regimen; and administering a dose of antihistamine to the subject from 30 to 60 minutes prior to the administration of the full dose during week 4 of the dosing regimen, and optionally administering a dose of acetaminophen to the subject from 30 to 60 minutes prior to the administration of the full dose during week 4 of the dosing regimen. In addition, in some embodiments, the dosing regimen further comprises administering a dose of steroid to the subject from 20 to 28 hours after the end of the administration of the full dose during week 4 of the dosing regimen.

In those embodiments of the dosing regimen in which the subject experiences a grade 3 or higher event of CRS when administered the initial dose, the first intermediate dose, or the second intermediate dose, and the full dose of the bispecific antibody administered to the subject during week 4 is split into two equal fractions (first and second fractions), then the dosing regimen further comprises: administering a dose of steroid to the subject from 12 to 24 hours prior to the administration of the first fraction of the full dose; administering a dose of steroid to the subject from 1 to 3 hours prior to the administration of the first fraction of the full dose; and administering a dose of antihistamine to the subject from 30 to 60 minutes prior to the administration of the first fraction of the full dose, and optionally administering a dose of acetaminophen to the subject from 30 to 60 minutes prior to the administration of the first fraction of the full dose.

In those instances in which the first fraction of the full dose and the second fraction of the full dose are not administered to the subject on consecutive days, then the dosing regimen further comprises administering a dose of steroid to the subject from 12 to 24 hours prior to the administration of the second fraction of the full dose of the bispecific antibody.

In addition, in those instances in which the subject experiences a grade 3 or higher event of CRS when administered the initial dose, the first intermediate dose, or the second intermediate dose, then the dosing regimen further comprises: administering a dose of steroid to the subject from 1 to 3 hours prior to the administration of the full dose during week 5 of the dosing regimen; and administering a dose of antihistamine to the subject from 30 to 60 minutes prior to the administration of the full dose during week 5 of the dosing regimen, and optionally administering a dose of acetaminophen to the subject from 30 to 60 minutes prior to the administration of the full dose during week 5 of the dosing regimen. In addition, in some embodiments, the dosing regimen further comprises administering a dose of steroid to the subject from 20 to 28 hours after the end of the administration of the full dose during week 5 of the dosing regimen.

In various embodiments, administering a dose of steroid, administering a dose of antihistamine, or administering a dose of acetaminophen comprises instructing the subject to ingest the dose of steroid, the dose of antihistamine, or the dose of acetaminophen, respectively. In some cases, the subject may orally self-administer the steroid, antihistamine or acetaminophen, and consequently the act of administering is merely an instruction to self-administer the medication. In other cases, administering a dose of steroid, or administering a dose of antihistamine comprises intravenously administering the dose of steroid or the dose of antihistamine. In some instances, the steroid or antihistamine may be administered by a physician, e.g., intravenously or by injection.

In various embodiments, the steroid is dexamethasone, and in some cases the dose is 20 mg. In other embodiments, the steroid is another equivalent steroid.

In various embodiments, the antihistamine is diphenhydramine, and the dose is 25 mg. In other embodiments, the antihistamine is another equivalent antihistamine.

In various embodiments, the dose of acetaminophen is 650 mg. In some cases, acetaminophen may not be administered as part of the dosing regimen if, for example, the subject has received or taken acetaminophen within 4 hours prior to the administration of the dose of bispecific antibody, or if the subject is allergic to acetaminophen.

In various embodiments, the dosing regimen further comprises administering anti-IL6 therapy (e.g., an anti-IL6 receptor antibody such as sarilumab or tocilizumab).

In those instances in which the subject does not experience any grade of IRR or CRS following the first full dose of the bispecific antibody as a single dose (i.e., at week 4 or week 5 of the dosing regimen), then with the following full dose the dose of steroid (e.g., dexamethasone) may be reduced to 10 mg. Similarly, if the subject does not experience any grade of IRR or CRS following a full dose with the reduced steroid dose (e.g., 10 mg dexamethasone), then subsequent doses of the bispecific antibody may be administered without premedication.

In any of the embodiments of the dosing regimen discussed above, the bispecific antibody may be as discussed herein, or below.

### Bispecific Antibodies

In various embodiments of the present disclosure, the bispecific antibody comprises A1-HCDR1, A1-HCDR2 and A1-HCDR3 comprising the amino acid sequences, respectively, of SEQ ID NOs: 7, 8 and 9; A2-HCDR1, A2-HCDR2 and A2-HCDR3 comprising the amino acid sequences, respectively, of SEQ ID NOs: 10, 11 and 12; and LCDR1, LCDR2 and LCDR3 comprising the amino acid sequences, respectively, of SEQ ID NOs: 13, 14 and 15.

In some embodiments, the first antigen-binding domain comprises a HCVR with at least 90% identity to the amino acid sequence of SEQ ID NO: 4 and a LCVR with at least 90% identity to the amino acid sequence of SEQ ID NO: 6, and the second antigen-binding domain comprises a HCVR with at least 90% identity to the amino acid sequence of SEQ ID NO: 5 and a LCVR with at least 90% identity to the amino acid sequence of SEQ ID NO: 6. In some embodiments, the first antigen-binding domain comprises a HCVR with at least 95% identity to the amino acid sequence of SEQ ID NO: 4 and a LCVR with at least 95% identity to the amino acid sequence of SEQ ID NO: 6, and the second antigen-binding domain comprises a HCVR with at least 95% identity to the amino acid sequence of SEQ ID NO: 5 and a LCVR with at least 95% identity to the amino acid sequence of SEQ ID NO: 6. In some embodiments, the first antigen-binding domain comprises a HCVR with at least 99% identity to the amino acid sequence of SEQ ID NO: 4 and a LCVR with at least 99% identity to the amino acid sequence of SEQ ID NO: 6, and the second antigen-binding domain comprises a HCVR with at least 99% identity to the amino acid sequence of SEQ ID NO: 5 and a LCVR with at least 99% identity to the amino acid sequence of SEQ ID NO: 6.

In various embodiments, the first antigen-binding domain comprises a HCVR comprising the amino acid sequence of SEQ ID NO: 4 and a LCVR comprising the amino acid sequence of SEQ ID NO: 6, and the second antigen-binding domain comprises a HCVR comprising the amino acid sequence of SEQ ID NO: 5 and a LCVR comprising the amino acid sequence of SEQ ID NO: 6.

The present invention also includes bispecific antibodies comprising a first C_{H}3 domain and a second Ig C_{H}3 domain, wherein the first and second Ig C_{H}3 domains differ from one another by at least one amino acid, and wherein at least one amino acid difference reduces binding of the bispecific antibody to Protein A as compared to a bi-specific antibody lacking the amino acid difference. In one embodiment, the first Ig C_{H}3 domain binds Protein A and the second Ig C_{H}3 domain contains a mutation that reduces or abolishes Protein A binding such as an H95R modification (by IMGT exon numbering; H435R by EU numbering). The second C_{H}3 may further comprise a Y96F modification (by IMGT; Y436F by EU). See, for example, U.S. Pat. No. 8,586,713. Further modifications that may be found within the second C_{H}3 include: D16E, L18M, N44S, K52N, V57M, and V821 (by IMGT; D356E, L358M, N384S, K392N, V397M, and V422I by EU) in the case of IgG1 antibodies; N44S, K52N, and V821 (IMGT; N384S, K392N, and V422I by EU) in the case of IgG2 antibodies; and Q15R, N44S, K52N, V57M, R69K, E79Q, and V821 (by IMGT; Q355R, N384S, K392N, V397M, R409K, E419Q, and V422I by EU) in the case of IgG4 antibodies.

In some embodiments, the bispecific antibody comprises a human IgG heavy chain constant region attached, respectively, to the HCVR of each of the first antigen-binding domain and the second antigen-binding domain. In some cases, the heavy chain constant region is of isotype IgG1. In some cases, the heavy chain constant region is of isotype IgG4. In some embodiments, the heavy chain constant region attached to the HCVR of the first antigen-binding domain or the heavy chain constant region attached to the HCVR of the second antigen-binding domain, but not both, contains an amino acid modification that reduces Protein A binding relative to a heavy chain of the same isotype without the modification. In some cases, the modification comprises a H435R substitution (EU numbering) in a heavy chain of isotype IgG1 or IgG4. In some cases, the modification comprises a H435R substitution and a Y436F substitution (EU numbering) in a heavy chain of isotype IgG1 or IgG4.

In certain embodiments, the Fc domain may be chimeric, combining Fc sequences derived from more than one immunoglobulin isotype. For example, a chimeric Fc domain can comprise part or all of a C_{H}2 sequence derived from a human IgG1, human IgG2 or human IgG4 C_{H}2 region, and part or all of a C_{H}3 sequence derived from a human IgG1, human IgG2 or human IgG4. A chimeric Fc domain can also contain a chimeric hinge region. For example, a chimeric hinge may comprise an “upper hinge” sequence, derived from a human IgG1, a human IgG2 or a human IgG4 hinge region, combined with a “lower hinge” sequence, derived from a human IgG1, a human IgG2 or a human IgG4 hinge region. A particular example of a chimeric Fc domain that can be included in any of the antigen-binding molecules set forth herein comprises, from N- to C-terminus: [IgG4 C_{H}1]-[IgG4 upper hinge]-[IgG2 lower hinge]-[IgG4 C_{H}2]-[IgG4 C_{H}3]. Another example of a chimeric Fc domain that can be included in any of the antigen-binding molecules set forth herein comprises, from N- to C-terminus: [IgG1 C_{H}1]-[IgG1 upper hinge]-[IgG2 lower hinge]-[IgG4 C_{H}2]-[IgG1 C_{H}3]. These and other examples of chimeric Fc domains that can be included in any of the antigen-binding molecules of the present invention are described in US Publication 2014/0243504, published Aug. 28, 2014, which is herein incorporated in its entirety. Chimeric Fc domains having these general structural arrangements, and variants thereof, can have altered Fc receptor binding, which in turn affects Fc effector function.

In some embodiments, the bispecific antibody comprises a chimeric hinge. For example, the chimeric hinge comprises, in an embodiment, a first amino acid sequence, or an “upper hinge” sequence, derived from a human IgG1 hinge region or human IgG4 hinge region, and a second amino acid sequence, or a “lower hinge” sequence, derived from a human IgG2 hinge region. In certain embodiments, the first or “upper hinge” sequence comprises amino acid residues from positions 216 to 227 according to EU numbering. In some embodiments, the second or “lower hinge” sequence comprises amino acid residues from positions 228 to 236 according to EU numbering. In some cases in which the antibody heavy chain constant region is of isotype IgG4, the chimeric hinge comprises an upper hinge sequence from human IgG4 (positions 216 to 227 according to EU numbering), and a lower hinge sequence from human IgG2 (positions 228 to 236 according to EU numbering). In some cases in which the antibody heavy chain constant region is of isotype IgG1, the chimeric hinge comprises an upper hinge sequence from human IgG1 (positions 216 to 227 according to EU numbering), and a lower hinge sequence from human IgG2 (positions 228 to 236 according to EU numbering). In some embodiments in which the heavy chain constant region is of isotype IgG1, and the antibody comprises a chimeric hinge, the C_{H}2 domain of the otherwise IgG1 heavy chain constant region is of isotype IgG4. Unless otherwise indicated, references to an IgG1 or IgG4 heavy chain constant region include heavy chain constant regions comprising a chimeric hinge (e.g., an IgG1 or IgG4 upper hinge sequence, respectively, and an IgG2 lower hinge sequence). For example, reference to an IgG1 heavy chain constant region includes an IgG1 heavy chain constant region that comprises an IgG2 lower hinge sequence, and reference to an IgG4 heavy chain constant region includes an IgG4 heavy chain constant region that comprises an IgG2 lower hinge sequence.

In some embodiments, the bispecific antibody comprises a heavy chain constant region comprising an amino acid sequence selected from the group consisting of SEQ ID NOs: 16, 17, 18 and 19. In some embodiments, the bispecific antibody comprises a heavy chain constant region comprising the amino acid sequence of SEQ ID NO: 16 and a heavy chain constant region comprising the amino acid sequence of SEQ ID NO: 17. In some embodiments, the bispecific antibody comprises a heavy chain constant region comprising the amino acid sequence of SEQ ID NO: 18 and a heavy chain constant region comprising the amino acid sequence of SEQ ID NO: 19.

In various embodiments, the bispecific antibody comprises a first heavy chain containing the HCVR of the first antigen-binding domain and a second heavy chain containing the HCVR of the second antigen-binding domain, wherein the first heavy chain comprises residues 1-452 of the amino acid sequence of SEQ ID NO: 1 and the second heavy chain comprises residues 1-448 of the amino acid sequence of SEQ ID NO: 2. In some embodiments, the antibody further comprises a common light chain containing the LCVR of the first and second antigen-binding domains, wherein the common light chain comprises the amino acid sequence of SEQ ID NO: 3.

In various embodiments of any of the pharmaceutical compositions discussed above or herein, the antibody comprises a first heavy chain containing the HCVR of the first antigen-binding domain and a second heavy chain containing the HCVR of the second antigen-binding domain, wherein the first heavy chain comprises the amino acid sequence of SEQ ID NO: 1 and the second heavy chain comprises the amino acid sequence of SEQ ID NO: 2. In some embodiments, the antibody further comprises a common light chain containing the LCVR of the first and second antigen-binding domains, wherein the common light chain comprises the amino acid sequence of SEQ ID NO: 3.

The first antigen-binding domain and the second antigen-binding domain may be directly or indirectly connected to one another to form a bispecific antibody of the present invention. Alternatively, the first antigen-binding domain and the second antigen-binding domain may each be connected to a separate multimerizing domain. The association of one multimerizing domain with another multimerizing domain facilitates the association between the two antigen-binding domains, thereby forming a bispecific antigen-binding molecule. A “multimerizing domain” is any macromolecule, protein, polypeptide, peptide, or amino acid that has the ability to associate with a second multimerizing domain of the same or similar structure or constitution. For example, a multimerizing domain may be a polypeptide comprising an immunoglobulin C_{H}3 domain. A non-limiting example of a multimerizing component is an Fc portion of an immunoglobulin (comprising a C_{H}2-C_{H}3 domain), e.g., an Fc domain of an IgG selected from the isotypes IgG1, IgG2, IgG3, and IgG4, as well as any allotype within each isotype group.

Bispecific antibodies of the present disclosure will typically comprise two multimerizing domains, e.g., two Fc domains that are each individually part of a separate antibody heavy chain. The first and second multimerizing domains may be of the same IgG isotype such as, e.g., IgG1/IgG1, IgG2/IgG2, IgG4/IgG4. Alternatively, the first and second multimerizing domains may be of different IgG isotypes such as, e.g., IgG1/IgG2, IgG1/IgG4, IgG2/IgG4, etc.

In certain embodiments, the multimerizing domain is an Fc fragment or an amino acid sequence of from 1 to about 200 amino acids in length containing at least one cysteine residue. In other embodiments, the multimerizing domain is a cysteine residue, or a short cysteine-containing peptide. Other multimerizing domains include peptides or polypeptides comprising or consisting of a leucine zipper, a helix-loop motif, or a coiled-coil motif.

### Binding Properties

The term “binding” in the context of the binding of an antibody (e.g., a bispecific antibody) to either, e.g., a predetermined antigen, such as a cell surface protein or fragment thereof, typically refers to an interaction or association between a minimum of two entities or molecular structures, such as an antibody-antigen interaction.

For instance, binding affinity typically corresponds to a K_{D} value of about 10⁻⁷ M or less, such as about 10⁻⁸ M or less, such as about 10⁻⁹ M or less when determined by, for instance, surface plasmon resonance (SPR) technology in a BIAcore 3000 instrument using the antigen as the ligand and the antibody, Ig, antibody-binding fragment, or Fc-containing protein as the analyte (or antiligand). Cell-based binding strategies, such as fluorescent-activated cell sorting (FACS) binding assays, are also routinely used, and FACS data correlates well with other methods such as radioligand competition binding and SPR (Benedict, C A, J Immunol Methods. 1997, 201(2):223-31; Geuijen, C A, et al. J Immunol Methods. 2005, 302(1-2):68-77).

Accordingly, the antibody or antigen-binding protein of the invention binds to the predetermined antigen or cell surface molecule (receptor) having an affinity corresponding to a K_{D} value that is at least ten-fold lower than its affinity for binding to a non-specific antigen (e.g., BSA, casein). According to the present invention, the affinity of an antibody corresponding to a K_{D} value that is equal to or less than ten-fold lower than a non-specific antigen may be considered non-detectable binding, however such an antibody may be paired with a second antigen binding arm for the production of a bispecific antibody of the invention.

The term “K_{D}” (M) refers to the dissociation equilibrium constant of a particular antibody-antigen interaction, or the dissociation equilibrium constant of an antibody or antibody-binding fragment binding to an antigen. There is an inverse relationship between K_{D} and binding affinity, therefore the smaller the K_{D} value, the higher, i.e. stronger, the affinity. Thus, the terms “higher affinity” or “stronger affinity” relate to a higher ability to form an interaction and therefore a smaller K_{D} value, and conversely the terms “lower affinity” or “weaker affinity” relate to a lower ability to form an interaction and therefore a larger K_{D} value. In some circumstances, a higher binding affinity (or K_{D}) of a particular molecule (e.g. antibody) to its interactive partner molecule (e.g. antigen X) compared to the binding affinity of the molecule (e.g. antibody) to another interactive partner molecule (e.g. antigen Y) may be expressed as a binding ratio determined by dividing the larger K_{D} value (lower, or weaker, affinity) by the smaller K_{D} (higher, or stronger, affinity), for example expressed as 5-fold or 10-fold greater binding affinity, as the case may be.

The term “k_{d}” (sec-1 or 1/s) refers to the dissociation rate constant of a particular antibody-antigen interaction, or the dissociation rate constant of an antibody or antibody-binding fragment. Said value is also referred to as the k_{off} value.

The term “kₐ” (M-1×sec-1 or 1/M) refers to the association rate constant of a particular antibody-antigen interaction, or the association rate constant of an antibody or antibody-binding fragment.

The term “K_{A}” (M-1 or 1/M) refers to the association equilibrium constant of a particular antibody-antigen interaction, or the association equilibrium constant of an antibody or antibody-binding fragment. The association equilibrium constant is obtained by dividing the kₐ by the k_{d}.

The term “EC50” or “EC₅₀” refers to the half maximal effective concentration, which includes the concentration of an antibody which induces a response halfway between the baseline and maximum after a specified exposure time. The EC₅₀ essentially represents the concentration of an antibody where 50% of its maximal effect is observed. In certain embodiments, the EC₅₀ value equals the concentration of an antibody of the invention that gives half-maximal binding to cells expressing, e.g., CD3 or a tumor-associated antigen (e.g., CD20), as determined by e.g. a FACS binding assay. Thus, reduced or weaker binding is observed with an increased EC₅₀, or half maximal effective concentration value.

In one embodiment, decreased binding can be defined as an increased EC₅₀ antibody concentration which enables binding to the half-maximal amount of target cells.

In another embodiment, the EC₅₀ value represents the concentration of an antibody of the invention that elicits half-maximal depletion of target cells by T cell cytotoxic activity. Thus, increased cytotoxic activity (e.g. T cell-mediated tumor cell killing) is observed with a decreased EC₅₀, or half maximal effective concentration value.

### Sequence Variants

The antibodies (e.g., bispecific antibodies) of the present disclosure may comprise one or more amino acid substitutions, insertions and/or deletions in the framework and/or CDR regions of the heavy and light chain variable domains as compared to the corresponding germline sequences from which the individual antigen-binding domains were derived. Such mutations can be readily ascertained by comparing the amino acid sequences disclosed herein to germline sequences available from, for example, public antibody sequence databases. The antigen-binding molecules of the present invention may comprise antigen-binding domains which are derived from any of the exemplary amino acid sequences disclosed herein, wherein one or more amino acids within one or more framework and/or CDR regions are mutated to the corresponding residue(s) of the germline sequence from which the antibody was derived, or to the corresponding residue(s) of another human germline sequence, or to a conservative amino acid substitution of the corresponding germline residue(s) (such sequence changes are referred to herein collectively as “germline mutations”). A person of ordinary skill in the art, starting with the heavy and light chain variable region sequences disclosed herein, can easily produce numerous antibodies and antigen-binding fragments which comprise one or more individual germline mutations or combinations thereof. In certain embodiments, all of the framework and/or CDR residues within the V_{H} and/or V_{L} domains are mutated back to the residues found in the original germline sequence from which the antigen-binding domain was originally derived. In other embodiments, only certain residues are mutated back to the original germline sequence, e.g., only the mutated residues found within the first 8 amino acids of FR1 or within the last 8 amino acids of FR4, or only the mutated residues found within CDR1, CDR2 or CDR3. In other embodiments, one or more of the framework and/or CDR residue(s) are mutated to the corresponding residue(s) of a different germline sequence (i.e., a germline sequence that is different from the germline sequence from which the antigen-binding domain was originally derived). Furthermore, the antigen-binding domains may contain any combination of two or more germline mutations within the framework and/or CDR regions, e.g., wherein certain individual residues are mutated to the corresponding residue of a particular germline sequence while certain other residues that differ from the original germline sequence are maintained or are mutated to the corresponding residue of a different germline sequence. Once obtained, antigen-binding domains that contain one or more germline mutations can be easily tested for one or more desired property such as, improved binding specificity, increased binding affinity, improved or enhanced antagonistic or agonistic biological properties (as the case may be), reduced immunogenicity, etc. Bispecific antigen-binding molecules comprising one or more antigen-binding domains obtained in this general manner are encompassed within the present invention.

The present invention also includes antibodies comprising variants of any of the HCVR, LCVR, and/or CDR amino acid sequences disclosed herein having one or more conservative substitutions. For example, the present invention includes antibodies having HCVR, LCVR, and/or CDR amino acid sequences with, e.g., 10 or fewer, 8 or fewer, 6 or fewer, 4 or fewer, etc. conservative amino acid substitutions relative to any of the HCVR, LCVR, and/or CDR amino acid sequences set forth herein.

pH-Dependent Binding

The present invention includes antibodies (e.g., bispecific antibodies) with pH-dependent binding characteristics. For example, an antibody of the present invention may exhibit reduced binding to, e.g., a tumor antigen such as CD20 at acidic pH as compared to neutral pH. Alternatively, antibodies of the invention may exhibit enhanced binding to, e.g., a tumor antigen such as CD20 at acidic pH as compared to neutral pH. The expression “acidic pH” includes pH values less than about 6.2, e.g., about 6.0, 5.95, 5.9, 5.85, 5.8, 5.75, 5.7, 5.65, 5.6, 5.55, 5.5, 5.45, 5.4, 5.35, 5.3, 5.25, 5.2, 5.15, 5.1, 5.05, 5.0, or less. The expression “neutral pH” means a pH of about 7.0 to about 7.4. The expression “neutral pH” includes pH values of about 7.0, 7.05, 7.1, 7.15, 7.2, 7.25, 7.3, 7.35, and 7.4.

In certain instances, “reduced binding . . . at acidic pH as compared to neutral pH” is expressed in terms of a ratio of the K_{D} value of the antibody binding to its antigen at acidic pH to the K_{D} value of the antibody binding to its antigen at neutral pH (or vice versa). For example, an antibody or antigen-binding fragment thereof may be regarded as exhibiting “reduced binding to, e.g., CD20 at acidic pH as compared to neutral pH” for purposes of the present invention if the antibody or antigen-binding fragment thereof exhibits an acidic/neutral K_{D} ratio of about 3.0 or greater. In certain exemplary embodiments, the acidic/neutral K_{D} ratio for an antibody or antigen-binding fragment of the present invention can be about 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0, 20.0. 25.0, 30.0, 40.0, 50.0, 60.0, 70.0, 100.0 or greater.

Antibodies with pH-dependent binding characteristics may be obtained, e.g., by screening a population of antibodies for reduced (or enhanced) binding to a particular antigen at acidic pH as compared to neutral pH. Additionally, modifications of the antigen-binding domain at the amino acid level may yield antibodies with pH-dependent characteristics. For example, by substituting one or more amino acids of an antigen-binding domain (e.g., within a CDR) with a histidine residue, an antibody with reduced antigen-binding at acidic pH relative to neutral pH may be obtained.

### Fc Variants

According to certain embodiments of the present invention, antibodies and bispecific antigen-binding molecules are provided comprising an Fc domain comprising one or more mutations which enhance or diminish antibody binding to the FcRn receptor, e.g., at acidic pH as compared to neutral pH. For example, the present invention includes antibodies comprising a mutation in the C_{H}2 or a C_{H}3 region of the Fc domain, wherein the mutation(s) increases the affinity of the Fc domain to FcRn in an acidic environment (e.g., in an endosome where pH ranges from about 5.5 to about 6.0). Such mutations may result in an increase in serum half-life of the antibody when administered to an animal. Non-limiting examples of such Fc modifications include, e.g., a modification at position 250 (e.g., E or Q); 250 and 428 (e.g., L or F); 252 (e.g., L/Y/F/W or T), 254 (e.g., S or T), and 256 (e.g., S/R/Q/E/D or T); or a modification at position 428 and/or 433 (e.g., H/L/R/S/P/Q or K) and/or 434 (e.g., H/F or Y); or a modification at position 250 and/or 428; or a modification at position 307 or 308 (e.g., 308F, V308F), and 434. In one embodiment, the modification comprises a 428L (e.g., M428L) and 434S (e.g., N434S) modification; a 428L, 2591 (e.g., V2591), and 308F (e.g., V308F) modification; a 433K (e.g., H433K) and a 434 (e.g., 434Y) modification; a 252, 254, and 256 (e.g., 252Y, 254T, and 256E) modification; a 250Q and 428L modification (e.g., T250Q and M428L); and a 307 and/or 308 modification (e.g., 308F or 308P).

For example, the present invention includes antibodies and bispecific antigen-binding molecules comprising an Fc domain comprising one or more pairs or groups of mutations selected from the group consisting of: 250Q and 248L (e.g., T250Q and M248L); 252Y, 254T and 256E (e.g., M252Y, S254T and T256E); 428L and 434S (e.g., M428L and N434S); and 433K and 434F (e.g., H433K and N434F). All possible combinations of the foregoing Fc domain mutations, and other mutations within the antibody variable domains disclosed herein, are contemplated within the scope of the present invention.

### Preparation of Antibodies

Antigen-binding domains specific for particular antigens can be prepared by any antibody generating technology known in the art. Once obtained, two different antigen-binding domains, specific for two different antigens (e.g., CD3 and CD20), can be appropriately arranged relative to one another to produce a bispecific antibody of the present disclosure using routine methods. In certain embodiments, one or more of the individual components (e.g., heavy and light chains) of the bispecific antibodies of the disclosure are derived from chimeric, humanized or fully human antibodies. Methods for making such antibodies are well known in the art. For example, one or more of the heavy and/or light chains of the bispecific antibodies of the present disclosure can be prepared using VELOCIMMUNE™ technology. Using VELOCIMMUNE™ technology (or any other human antibody generating technology), high affinity chimeric antibodies to a particular antigen (e.g., CD3 or CD20) are initially isolated having a human variable region and a mouse constant region. The antibodies are characterized and selected for desirable characteristics, including affinity, selectivity, epitope, etc. The mouse constant regions are replaced with a desired human constant region to generate fully human heavy and/or light chains that can be incorporated into the bispecific antigen-binding molecules of the present invention.

Genetically engineered animals may be used to make human bispecific antigen-binding molecules. For example, a genetically modified mouse can be used which is incapable of rearranging and expressing an endogenous mouse immunoglobulin light chain variable sequence, wherein the mouse expresses only one or two human light chain variable domains encoded by human immunoglobulin sequences operably linked to the mouse kappa constant gene at the endogenous mouse kappa locus. Such genetically modified mice can be used to produce fully human bispecific antibodies comprising two different heavy chains that associate with an identical light chain that comprises a variable domain derived from one of two different human light chain variable region gene segments. (See, e.g., US 2011/0195454). Fully human refers to an antibody, or antigen-binding fragment or immunoglobulin domain thereof, comprising an amino acid sequence encoded by a DNA derived from a human sequence over the entire length of each polypeptide of the antibody or antigen-binding fragment or immunoglobulin domain thereof. In some instances, the fully human sequence is derived from a protein endogenous to a human. In other instances, the fully human protein or protein sequence comprises a chimeric sequence wherein each component sequence is derived from human sequence. While not being bound by any one theory, chimeric proteins or chimeric sequences are generally designed to minimize the creation of immunogenic epitopes in the junctions of component sequences, e.g. compared to any wild-type human immunoglobulin regions or domains.

### Bioequivalents

The present invention encompasses antigen-binding molecules having amino acid sequences that vary from those of the exemplary molecules disclosed herein but that retain the ability to bind the same antigen or antigens. Such variant molecules may comprise one or more additions, deletions, or substitutions of amino acids when compared to parent sequence, but exhibit biological activity that is essentially equivalent to that of the described antibodies (e.g., bispecific antibodies).

The present invention includes antigen-binding molecules that are bioequivalent to any of the exemplary antibodies set forth herein. Two antigen-binding proteins, or antibodies, are considered bioequivalent if, for example, they are pharmaceutical equivalents or pharmaceutical alternatives whose rate and extent of absorption do not show a significant difference when administered at the same molar dose under similar experimental conditions, either single dose or multiple dose. Some antigen-binding proteins will be considered equivalents or pharmaceutical alternatives if they are equivalent in the extent of their absorption but not in their rate of absorption and yet may be considered bioequivalent because such differences in the rate of absorption are intentional and are reflected in the labeling, are not essential to the attainment of effective body drug concentrations on, e.g., chronic use, and are considered medically insignificant for the particular drug product studied.

In one embodiment, two antigen-binding proteins are bioequivalent if there are no clinically meaningful differences in their safety, purity, and potency.

In one embodiment, two antigen-binding proteins are bioequivalent if a patient can be switched one or more times between the reference product and the biological product without an expected increase in the risk of adverse effects, including a clinically significant change in immunogenicity, or diminished effectiveness, as compared to continued therapy without such switching.

In one embodiment, two antigen-binding proteins are bioequivalent if they both act by a common mechanism or mechanisms of action for the condition or conditions of use, to the extent that such mechanisms are known.

Bioequivalence may be demonstrated by in vivo and in vitro methods. Bioequivalence measures include, e.g., (a) an in vivo test in humans or other mammals, in which the concentration of the antibody or its metabolites is measured in blood, plasma, serum, or other biological fluid as a function of time; (b) an in vitro test that has been correlated with and is reasonably predictive of human in vivo bioavailability data; (c) an in vivo test in humans or other mammals in which the appropriate acute pharmacological effect of the antibody (or its target) is measured as a function of time; and (d) in a well-controlled clinical trial that establishes safety, efficacy, or bioavailability or bioequivalence of an antigen-binding protein.

Bioequivalent variants of the exemplary antibodies (e.g., bispecific antibodies) set forth herein may be constructed by, for example, making various substitutions of residues or sequences or deleting terminal or internal residues or sequences not needed for biological activity. For example, cysteine residues not essential for biological activity can be deleted or replaced with other amino acids to prevent formation of unnecessary or incorrect intramolecular disulfide bridges upon renaturation. In other contexts, bioequivalent antigen-binding proteins may include variants of the exemplary antibodies and bispecific antigen-binding molecules set forth herein comprising amino acid changes which modify the glycosylation characteristics of the molecules, e.g., mutations which eliminate or remove glycosylation.

### Therapeutic Formulation and Delivery

The present invention provides pharmaceutical compositions comprising the antibodies (e.g., bispecific antibodies) of the present disclosure. Specific pharmaceutical compositions of exemplary bispecific antibodies of the present disclosure are provided in, e.g., WO 2021/119135.

Various delivery systems are known and can be used to administer the pharmaceutical composition of the invention, e.g., encapsulation in liposomes, microparticles, microcapsules, recombinant cells capable of expressing the mutant viruses, receptor mediated endocytosis (see, e.g., Wu et al., 1987, J. Biol. Chem. 262:4429-4432). Methods of introduction include, but are not limited to, intradermal, intramuscular, intraperitoneal, intravenous, subcutaneous, intranasal, epidural, and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In some embodiments, the therapeutic proteins of the invention are administered via intravenous infusion or subcutaneous injection.

A pharmaceutical composition of the present invention can be delivered subcutaneously or intravenously with a standard needle and syringe. In addition, with respect to subcutaneous delivery, a pen delivery device readily has applications in delivering a pharmaceutical composition of the present invention. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

Numerous reusable pen and autoinjector delivery devices have applications in the subcutaneous delivery of a pharmaceutical composition of the present invention. Examples include, but are not limited to AUTOPEN™ (Owen Mumford, Inc., Woodstock, UK), DISETRONIC™ pen (Disetronic Medical Systems, Bergdorf, Switzerland), HUMALOG MIX 75/25™ pen, HUMALOG™ pen, HUMALIN 70/30™ pen (Eli Lilly and Co., Indianapolis, Ind.), NOVOPEN™ I, II and III (Novo Nordisk, Copenhagen, Denmark), NOVOPEN JUNIOR™ (Novo Nordisk, Copenhagen, Denmark), BD™ pen (Becton Dickinson, Franklin Lakes, N.J.), OPTIPEN™, OPTIPEN PRO™, OPTIPEN STARLET™, and OPTICLIK™ (sanofi-aventis, Frankfurt, Germany), to name only a few. Examples of disposable pen delivery devices having applications in subcutaneous delivery of a pharmaceutical composition of the present invention include, but are not limited to the SOLOSTAR™ pen (sanofi-aventis), the FLEXPEN™ (Novo Nordisk), and the KWIKPEN™ (Eli Lilly), the SURECLICK™ Autoinjector (Amgen, Thousand Oaks, Calif.), the PENLET™ (Haselmeier, Stuttgart, Germany), the EPIPEN (Dey, L.P.), and the HUMIRA™ Pen (Abbott Labs, Abbott Park Ill.), to name only a few.

The injectable preparations may include dosage forms for intravenous, subcutaneous, intracutaneous and intramuscular injections, drip infusions, etc. These injectable preparations may be prepared by methods publicly known.

The pharmaceutical compositions of the present disclosure may be included in a kit comprising a container containing the bispecific antibody and a label with instructions to administer the bispecific antibody according to any one or more of the dosing regimens discussed herein. In some case, the label also includes instructions for administration of steroids and/or antihistamines to mitigate the risk of CRS and/or IRR.

### Therapeutic Uses of the Antigen-Binding Molecules

The present invention includes methods comprising administering to a subject in need thereof a therapeutically effective amount of the bispecific antibodies of the present disclosure for the treatment of B-cell malignancies. The bispecific antibodies may be contained in a composition comprising a pharmaceutically acceptable carrier or diluent. The expressions “a subject” or “a subject in need thereof” mean a human or non-human animal that exhibits one or more symptoms or indicia of cancer (e.g., a subject expressing a tumor or suffering from any of the cancers mentioned herein below).

In some embodiments, the bispecific anti-CD3 x anti-CD20 antibodies are useful for treating a B-cell malignancy including non-Hodgkin lymphoma, Hodgkin lymphoma, chronic lymphocytic leukemia, acute lymphoblastic leukemia, small lymphocytic lymphoma, diffuse large B-cell lymphoma, follicular lymphoma, mantle cell lymphoma, marginal zone lymphoma, Waldenstrom macroglobulinemia, primary mediastinal B-cell lymphoma, lymphoblastic lymphoma, or Burkitt lymphoma. In some embodiments, the cancer is follicular lymphoma. In some embodiments, the cancer is diffuse large B-cell lymphoma (DLBCL). In some embodiments, the cancer is mantle cell lymphoma. In some embodiments, the cancer is marginal zone lymphoma.

Non-Hodgkin Lymphoma (NHL) is the most common hematological malignancy. Among a heterogeneous group of NHLs, 85-90% are of B-cell origin and include follicular lymphoma (FL), diffuse large B-cell lymphoma (DLBCL), mantle cell lymphoma (MCL), marginal zone lymphoma (MZL), and several other B-NHLs. Anti-CD20 antibodies in combination with chemotherapy are the standard of care for the treatment of B-NHLs; however, despite initial responses, many patients relapse, often with progressively shorter response durations in subsequent lines of therapy and poor outcomes. Thus, in some embodiments, the antigen-binding molecule is a bispecific anti-CD3×anti-CD20 that binds to CD3+ T cells and CD20+B cells, targeting CD20+ tumor cells via T-cell mediated cytotoxicity. In some cases, the anti-CD3 x CD20 bispecific antibody is for treatment of a B-cell cancer (e.g., a NHL) in a subject that has failed prior therapy with an anti-CD20 monospecific antibody.

For patients with less than a complete response to CAR-T therapy, the outcomes are generally poor, and there are no standard-of-care therapeutic options. Thus, in some cases, the anti-CD3 x CD20 bispecific antibody of the present invention is for treatment of a B-cell cancer (e.g., a NHL such as DLBCL) in a subject that has failed prior CAR-T therapy or is not responsive to prior CAR-T therapy (e.g., anti-CD19 CAR-T therapy).

In an embodiment, odronextamab is indicated for the treatment of adult patients with relapsed or refractory follicular lymphoma (FL) after at least two prior systemic therapies.

In an embodiment, odronextamab is indicated for the treatment of adult patients diagnosed with follicular lymphoma or DLBCL who have not been previously treated with any systemic anti-lymphoma therapy.

For relapsed or refractory follicular lymphoma (R/R FL), administration may be via an intravenous (IV) infusion. Treatment consists of step-up dosing in cycle 1, weekly dosing in cycles 2-4 followed by maintenance dosing every 2 weeks until disease progression, as for example, shown in Table 1, below.

A single treatment cycle (for FL) consists of 21 days.

Cycle 1: Step Up—Administer odronextamab as a 4 hour infusion. The recommended starting dose of odronextamab is 0.2 mg on day 1. If tolerated, administer 0.5 mg on day 2. If tolerated, administer a dose of 2 mg on day 8 and 2 mg on day 9. If tolerated, administer a dose of 10 mg on day 15 and 10 mg on day 16. If tolerated, administer cycle 2.
Cycles 2-4: 80 mg Weekly—Administer 80 mg as a 4 hour infusion on cycle 2, day 1. If tolerated, infusion time can be reduced to 1 hour for all subsequent doses. Administer a dose of 80 mg on day 1, day 8 and day 15.
Maintenance: 160 mg Every 2 Weeks—After cycle 4, administer odronextamab at a dose of 160 mg as a 1 hour infusion every two weeks. If a patient is in complete remission for 9 months, administer a dose of 160 mg every 4 weeks, or 320 mg every 8 weeks.

TABLE 1
Recommended Odronextamab Dose and Schedule for the Treatment of R/R FL

| | | Dose of IV | |
|---|---|---|---|
| Day of Treatment Cycle | | Odronextamab (mg) | Administration Instructions |
| Cycle 1 | Day 1 | 0.2 | Administer as a 4 hour infusion |
| (Step Up) | Day 2^{a} | 0.5 | |
| | Day 8 | 2 | |
| | Day 9^{b} | 2 | |
| | Day 15 | 10 | |
| | Day 16^{c} | 10 | |
| Cycles 2-4 | Day 1 | 80 | Administer 80 mg as a 4 hour |
| | Day 8 | 80 | infusion on cycle 2, day 1. If |
| | Day 15 | 80 | tolerated, infusion time can be |
| | | | reduced to 1 hour for all |
| | | | subsequent doses. |
| Maintenance | Begin 2 weeks | 160 | Administer as a 1 hour infusion. |
| (Every 2 Weeks) | after Cycle 4, | | If a patient is in complete |
| | Day 15. | | remission for 9 months, |
| | | | administer a dose of 160 mg |
| | | | every 4 weeks. |

^{a}Dose on Day 2 can administered on Day 2, 3 or 4.
^{b}Dose on Day 9 can administered on Day 9, 10 or 11.
^{c}Dose on Day 16 can administered on Day 16, 17 or 18.

In an embodiment, odronextamab is indicated for the treatment of adult patients with relapsed or refractory diffuse large B-cell lymphoma (DLBCL) after at least two prior systemic therapies.

For diffuse large B-cell lymphoma (DLBCL), administration may be via an intravenous (IV) infusion. Treatment consists of step-up dosing in cycle 1, weekly dosing in cycles 2-4 followed by maintenance dosing every 2 weeks until disease progression, as for example, shown in Table 2, below.

A single treatment cycle (for DLBCL) consists of 21 days.

Cycle 1: Step Up—Administer odronextamab as a 4 hour infusion. The recommended starting dose of odronextamab is 0.2 mg on day 1. If tolerated, administer 0.5 mg on day 2. If tolerated, administer a dose of 2 mg on day 8 and 2 mg on day 9. If tolerated, administer a dose of 10 mg on day 15 and 10 mg on day 16. If tolerated, administer cycle 2.
Cycles 2-4: 160 mg Weekly—Administer 160 mg as a 4 hour infusion on cycle 2, day 1. If tolerated, infusion time can be reduced to 1 hour for all subsequent doses. Administer a dose of 160 mg on day 1, day 8 and day 15.
Maintenance: 320 mg Every 2 Weeks—After cycle 4, administer odronextamab at a dose of 320 mg as a 1 hour infusion every two weeks. If a patient is in complete remission for 9 months, administer a dose of 320 mg every 4 weeks.

TABLE 2
Recommended Odronextamab Dose and Schedule for the Treatment of R/R DLBCL

| | | Dose of IV | |
|---|---|---|---|
| Day of Treatment Cycle | | Odronextamab (mg) | Administration Instructions |
| Cycle 1 | Day 1 | 0.2 | Administer as a 4 hour infusion |
| (Step Up) | Day 2^{a} | 0.5 | |
| | Day 8 | 2 | |
| | Day 9^{b} | 2 | |
| | Day 15 | 10 | |
| | Day 16^{c} | 10 | |
| Cycles 2-4 | Day 1 | 160 | Administer 80 mg as a 4 hour |
| | Day 8 | 160 | infusion on cycle 2, day 1. If |
| | Day 15 | 160 | tolerated, infusion time can be |
| | | | reduced to 1 hour for all |
| | | | subsequent doses. |
| Maintenance | Begin 2 weeks | 320 | Administer as a 1 hour infusion. |
| (Every 2 Weeks) | after Cycle 4, | | If a patient is in complete |
| | Day 15. | | remission for 9 months, |
| | | | administer a dose of 160 mg |
| | | | every 4 weeks. |

^{a}Dose on Day 2 can administered on Day 2, 3 or 4.
^{b}Dose on Day 9 can administered on Day 9, 10 or 11.
^{c}Dose on Day 16 can administered on Day 16, 17 or 18.

In the indications referenced above, or herein, administration of premedications, as detailed in Table 3A below, may be desirable to reduce the risk of cytokine release syndrome (CRS) and/or infusion-related reactions (IRR). Table 3B describes specific options for management of CRS.

TABLE 3A
Premedications

| | | Administration Relative to |
|---|---|---|
| Day of treatment cycle | Premedications | Odronextamab Infusion |
| Cycle 1: | 10 mg dexamethasone oral or | 12-24 hours prior to infusion |
| Days 1, 2, 8, 9, 15, 16 | equivalent dose of steroid | Repeat if the second infusion was |
| Cycle 2: | | on non-consecutive days. |
| Day 1 | 20 mg dexamethasone IV | 1-3 hours prior to infusion |
| | Anti-histamine (e.g. | 30-60 minutes prior to infusion |
| | diphenhydramine 25 mg oral or | |
| | IV) | |
| Cycle 1: | Acetaminophen 650 mg oral | 30-60 minutes prior to infusion |
| Day 3, 10, 17 | 10 mg dexamethasone oral or | 24 hours after infusion |
| Cycle 2: | equivalent dose of steroid | |
| Day 2 | | |
| Cycle 2: | 10 mg dexamethasone IV | 1-3 hours prior to infusion |
| Day 8 | Anti-histamine (e.g. | 30-60 minutes prior to infusion |
| | diphenhydramine 25 mg oral or | |
| | IV) | |
| | Acetaminophen 650 mg oral | 30-60 minutes prior to infusion |

TABLE 3B
Grading and Management Guidelines for CRS

| CRS Grade | Corticosteroids | Tocilizumab | Other |
|---|---|---|---|
| Grade 1 CRS | Administer | If not improving after 3 | Interrupt |
| Fever ≥38° C. | dexamethasone 10 | days, consider | administration of |
| | mg IV (or equivalent | managing as Grade 2 | bispecific antibody, |
| | corticosteroid) every | | and resume when |
| | 24 hours until | | symptoms resolve. |
| | resolution | | |
| Grade 2 CRS | Administer | Administer tocilizumab | Interrupt |
| Fever ≥38° C. | dexamethasone 10 | 8 mg/kg intravenously | administration of |
| Hypoxia requiring low | mg IV (or equivalent | over 1 hour (not to | bispecific antibody, |
| flow oxygen (≤6 | corticosteroid) every | exceed 800 mg) | and resume when |
| L/min.) by nasal | 12-24 hours | If no clinical | symptoms resolve. |
| cannula, or | If no improvement or | improvement in the | |
| hypotension | rapid progression, | signs and symptoms | |
| responsive to fluids | increase | of CRS after the first | |
| | dexamethasone to 10- | dose, repeat | |
| | 20 mg IV (or | tocilizumab every 8 | |
| | equivalent | hours as needed | |
| | corticosteroid) every | Limit to a maximum of | |
| | 6-12 hours | 3 doses in a 24-hour | |
| | | period, maximum total | |
| | | of 4 doses | |
| Grade 3 CRS | Administer | Per Grade 2 | Interrupt |
| Fever ≥38° C. | corticosteroid) every | | and resume when |
| Hypoxia requiring | 6-12 hours | | symptoms resolve. |
| positive pressure | dexamethasone 10-20 | | administration of |
| ventilation and/or | mg IV (or equivalent | | bispecific antibody, |
| hypotension requiring | | | |
| use of multiple | | | |
| vasopressoers | | | |

Premedications may be continued beyond cycle 2, day 8 until the dose is tolerated without experiencing IRR and/or CRS. Earlier use of anti-IL6 therapy (e.g., tocilizumab) is recommended for patients >65 years of age, or for patients with co-morbidities.

### Combination Therapies

The present invention provides methods which comprise administering a pharmaceutical composition comprising any of the exemplary antibodies (e.g., bispecific antibodies) described herein in combination with one or more additional therapeutic agents. Exemplary additional therapeutic agents that may be combined with or administered in combination with an antigen-binding molecule of the present invention include, e.g., an anti-tumor agent (e.g. chemotherapeutic agents as disclosed elsewhere herein). In certain embodiments the additional therapeutic agent is a regimen comprising radiotherapy or a hematopoietic stem cell transplant. In certain embodiments, the additional therapeutic agent may be an immunomodulatory agent. In certain embodiments, the additional therapeutic agent may be a monoclonal antibody, an antibody drug conjugate, a bispecific antibody conjugated to an anti-tumor agent, an immune checkpoint inhibitor (e.g., PD-1 or CTLA-4, or combinations thereof), or combinations thereof.

The additional therapeutically active component(s) may be administered just prior to, concurrent with, or shortly after the administration of an antigen-binding molecule of the present invention; (for purposes of the present disclosure, such administration regimens are considered the administration of an antigen-binding molecule “in combination with” an additional therapeutically active component).

The present invention includes pharmaceutical compositions in which an antigen-binding molecule of the present invention is co-formulated with one or more of the additional therapeutically active component(s) as described elsewhere herein.

### Sequences

A summary of the sequences and the corresponding SEQ ID NOs referenced herein is shown in Table 4, below. The anti-CD3 x anti-CD20 antibody comprising the heavy chains and common light chain of SEQ ID NOs: 1-3, the HCVRs and LCVR of SEQ ID NOs: 4-6, and the CDRs of SEQ ID NOs: 7-15, is also referred to herein as odronextamab.

TABLE 4
Summary of Sequences

| SEQ ID NO: | Description |
|---|---|
| 1 | Anti-CD20 Heavy Chain |
| 2 | Anti-CD3 Heavy Chain |
| 3 | Common Anti-CD20 and Anti-CD3 Light Chain |
| 4 | Anti-CD20 HCVR |
| 5 | Anti-CD3 HCVR |
| 6 | Common Anti-CD20 and Anti-CD3 LCVR |
| 7 | Anti-CD20 HCDR1 |
| 8 | Anti-CD20 HCDR2 |
| 9 | Anti-CD20 HCDR3 |
| 10 | Anti-CD3 HCDR1 |
| 11 | Anti-CD3 HCDR2 |
| 12 | Anti-CD3 HCDR3 |
| 13 | Common Anti-CD20 and Anti-CD3 LCDR1 |
| 14 | Common Anti-CD20 and Anti-CD3 LCDR2 |
| 15 | Common Anti-CD20 and Anti-CD3 LCDR3 |
| 16 | IgG4 Heavy Chain Constant Region |
| 17 | IgG4 Heavy Chain Constant Region with H435R/Y436F |
| 18 | IgG1 Heavy Chain Constant Region |
| 19 | IgG1 Heavy Chain Constant Region with H435R/Y436F |

### EXAMPLES

The following examples are put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1: Clinical Evaluation of Anti-CD3 x Anti-CD20 Bispecific Antibody in Patients with CD20+ B-Cell Malignancies

This is an open-label, multi-center, dose escalation study of odronextamab (anti-CD3 x anti-CD20) administered as an IV infusion. The study consists of a dose escalation portion for B-NHL and CLL cohorts, and disease-specific expansions for DLBCL after failure of CAR-T therapy, aggressive lymphoma (excluding prior CAR-T therapy), and follicular lymphoma grade 1-3a.

The treatment duration will comprise 12 once a week (QW) doses followed by dosing every two weeks (Q2W) through the time of disease progression or other protocol-defined reason for treatment discontinuation. In addition, if a patient has demonstrated a complete response (CR) and has shown a durable response for at least 9 months after the initial determination of CR, then the frequency of study drug administration at the assigned dose will be decreased from Q2W to every four weeks (Q4W) intervals.

Patients will receive odronextamab at an initial dose of 0.7 mg during treatment week 1 (administered as a split dose of 0.2 mg/0.5 mg), followed by an intermediate dose-1 of 4 mg at treatment week 2 (administered as a split dose of 2 mg/2 mg), an intermediate dose-2 of 20 mg at treatment week 3 (administered as a split dose of 10 mg/10 mg) and then the assigned full QW dose at treatment week 4 through 12 followed by Q2W dosing. The initial dose (0.7 mg) and intermediate dose-1 of 4 mg and intermediate dose-2 of 20 mg are always administered as a split infusion over 2 days, preferably consecutive but no more than 3 days apart, even if these doses were delayed beyond treatment week 3. Administration of the QW dose will only proceed if the full initial and full intermediate dose 1 and 2 were received and tolerated. If a patient does not experience grade 3 CRS with the initial dose and the intermediate doses, then at treatment week 4 and beyond, QW dose of odronextamab will be administered as a single infusion. However, if a patient experiences grade 3 CRS with the initial dose or the intermediate doses, the first odronextamab QW dose will be administered as a split infusion over 2 days.

Premedication are required prior to the start of odronextamab infusion for each split initial dose, each split intermediate dose, each split QW dose (if applicable), and the first administration of the QW dose as a single infusion; premedications will be administered as detailed in Example 3. If no IRR/CRS of any grade are experienced following the first QW dose administered as a single infusion, investigators may initiate tapering of the dexamethasone premedication over subsequent administrations of odronextamab at the week 5 dose, as detailed in Example 3. The implementation of enhanced tocilizumab use will depend on the rate of grade CRS events observed at any time.

Patients will receive odronextamab QW at the assigned dose during a 4-week induction period, followed by an additional 8 QW doses, and Q2W treatment at the assigned dose until the time of disease progression or other protocol-defined reason for treatment discontinuation. If a patient has demonstrated a CR and has shown a durable response for at least 9 months after the initial determination of CR, then the frequency of study drug administration at the assigned dose will be decreased from Q2W to Q4W intervals. Patients must be receiving the full dose of Q2W dosing for at least 3 preceding doses before switching from Q2W to Q4W dosing.

### Inclusion Criteria

A patient must meet the following criteria to be eligible for inclusion in the study:
1. Have documented CD20+ B-cell malignancy, with active disease not responsive to prior therapy, for whom no standard of care options exists, and for whom treatment with an anti-CD20 antibody may be appropriate:
   B-NHL confirmed by NCI working group criteria, 2007
   CLL confirmed by the International Workshop on Chronic Lymphocytic Leukemia (IWCLL) working group criteria, 2008
      Patients with small lymphocytic lymphoma (SLL) will be enrolled in the CLL arm and follow B-NHL assessments
   NOTE: A patient with CD20-negative lymph node (NHL) biopsy performed as standard of care just prior to enrollment, remains eligible for the study provided the patient had previously documented CD20+ disease AND was previously treated with rituximab or other CD20-directed antibody therapy within approximately 6 months.
2. Patients with B-NHL must have had prior treatment with an anti-CD20 antibody therapy. Patients with CLL are not required to have received prior treatment with an anti-CD20 antibody therapy, provided the patient has failed either a BTK inhibitor or PI3K inhibitor and the treating physician deems it appropriate for the patient to be entered into a phase 1 trial. Refractory is defined as no response (SD/PD) or relapse within months of last treatment.
   For inclusion in the FL grade 1-3a expansion cohort, patients must have received at least 2 prior lines of systemic therapy, including an anti-CD20 antibody and an alkylating agent; patients require failure of combination of lenalidomide plus rituximab where approved or not appropriate to receive this treatment according to the investigator.
   For the inclusion in the disease-specific expansion cohort enrolling DLBCL patients after failure of CAR-T therapy, the patient must have recovered from the toxicities of the lymphodepletion therapy and CAR-T infusion. There is no requirement for the prior CAR-T therapy to be the most recent line of therapy before study enrollment.
   Patients with aggressive lymphoma assigned to the aggressive lymphoma expansion cohort must have received at least one prior line of therapy consisting of an anti-CD20 antibody. Patients with prior CAR-T therapy will not be included in this cohort.
   Patients with lymphoma eligible for the aggressive lymphoma expansion cohort include the following subtypes based on the WHO classification:
      DLBCL not otherwise specified (NOS)
      Germinal center B-cell type
      Activated B-cell type
   (Note: DLBCL includes both de novo DLBCL and transformed DLBCL arising from indolent lymphoma/CLL)
   Primary mediastinal (thymic) large B-cell lymphoma
   T-cell/histiocyte-rich large B-cell lymphoma
   Epstein-Barr virus (EBV)+ DLBCL, NOS
   High-grade B-cell lymphoma, with MYC and BCL2 and/or BCL6 rearrangements
   High-grade B-cell lymphoma, NOS
   B-cell lymphoma, unclassifiable, with features intermediate between DLBCL and classical Hodgkin lymphoma
   Follicular lymphoma, grade 3b
3. All patients (B-cell NHL and CLL) must have at least one bi-dimensionally measurable lesion ≥1.5 cm on the longest diameter) documented by CT or MRI scan, if CT scan is not feasible.
4. Patients with CLL must have white blood cell (WBC)≤200×109/L. As of protocol amendment 16, enrollment is closed for patients with CLL.
5. Age≥18 years
6. Eastern Cooperative Oncology Group (ECOG) performance status ≤1
7. Life expectancy of at least 6 months
8. Adequate bone marrow function documented by:
   a. Platelet counts ≥75×10⁹/L
   b. Hb level ≥9 g/dL
   c. ANC≥1×10⁹/L
   NOTE: Patients with cell counts below thresholds listed above may be considered for enrollment if, in the opinion of the investigator, the reason is believed to be due to bone marrow infiltration or splenic sequestration by underlying disease.
   Patients with bone marrow involvement or splenic sequestration should meet the following hematologic parameters:
      Platelet count ≥25×109/L. A patient may not have received platelet transfusion therapy within 3 days prior to first dose of odronextamab in order to meet the platelet eligibility criterion.
      Hemoglobin ≥7.0 g/dL
      Absolute neutrophil count (ANC)≥0.5×109/L. A patient may not have received granulocyte colony stimulating factor within 2 days prior to first dose of odronextamab in order to meet the ANC eligibility criterion.
9. Adequate organ function documented by:
   Aspartate aminotransferase (AST) and alanine aminotransferase (ALT)≤2.5×ULN
   Total bilirubin ≤1.5×ULN
   NOTE: Patients with Gilbert's syndrome do not need to meet this requirement provided their total bilirubin is unchanged from their baseline.
   Calculated creatinine clearance by Cockcroft-Gault 50 mL/min
   NOTE: Patients may be considered for enrollment if, in the opinion of the investigator, the abnormal laboratory results are due to underlying disease.
   NOTE: Patients with borderline creatinine clearance by Cockcroft-Gault may be considered for enrollment if a measured creatinine clearance (based on 24-hour urine or other reliable method) is ≥50 mL/min.
10. Willingness to undergo mandatory tumor biopsy pre-treatment, if in the opinion of the investigator, the patient has an accessible lesion that can be biopsied without significant risk to the patient.
11. Willing and able to comply with clinic visits and study-related procedures
12. Provide signed informed consent or legally acceptable representative

Exclusion Criteria

A patient who meets any of the following criteria will be excluded from the study:
1. Primary central nervous system (CNS) lymphoma or known or suspected CNS involvement by non-primary CNS NHL
2. History of or current relevant CNS pathology such as:
   Epilepsy, seizure, paresis, aphasia, apoplexia, severe brain injuries, cerebellar disease, organic brain syndrome, psychosis, or
   Evidence for presence of inflammatory lesions and/or vasculitis on cerebral MRI
3. Standard anti-neoplastic chemotherapy (non-biologic) within 5-times the half-life or within 28 days, whichever is shorter, prior to first administration of study drug.
4. Standard radiotherapy within 14 days of first administration of study drug.
   NOTE: Palliative radiotherapy to a symptomatic lymph node/lesion is allowed provided the irradiated lesion(s) or node(s) is not included as a target lesion for tumor assessments
5. Allogeneic stem cell transplantation
6. Treatment with rituximab, alemtuzumab, or other investigational or commercial biologic agent within 12 weeks prior to first administration of study drug.
   NOTE: for patients with aggressive lymphoma for which immediate treatment is required, the wash-out period may be reduced to 28 days.
7. Immunosuppressive therapy (other than biologic) within 28 days of first administration of study drug.
8. Treatment with an investigational non-biologic agent within 28 days of first administration of study drug.
9. History of allergic reactions attributed to compounds of similar chemical or biologic composition of study drug.
10. History of hypersensitivity to any compound in the tetracycline antibiotics group.
11. Concurrent active malignancy for which the patient is receiving treatment.
12. Known active bacterial, viral, fungal, mycobacterial or other infection or any major episode of infection requiring hospitalization or treatment with IV anti-infectives within 4 weeks of first administration.
13. Evidence of significant concurrent disease or medical condition that could interfere with the conduct of the study, or put the patient at significant risk including, but not limited to, significant cardiovascular disease (eg, New York Heart Association Class III or IV cardiac disease, myocardial infarction within the previous 6 months, unstable arrhythmias or unstable angina) and/or significant pulmonary disease (eg, obstructive pulmonary disease and history of symptomatic bronchospasm).
   NOTE: Patients with a medical history of cardiac disease should be evaluated by ECHO or multi-gated acquisition scan (MUGA) prior to first administration of odronextamab to ensure adequate cardiac reserves and function.
14. Ongoing systemic corticosteroid treatment, with the exception of corticosteroid use for other (non-tumor and non-immunosuppressive) indications up to a maximum of 10 mg/day of prednisone or equivalent.
15. Infection with human immunodeficiency virus (HIV) or chronic infection with hepatitis B virus (HBV) or hepatitis C virus (HCV).
   Patients with hepatitis B (HepBsAg+) who have controlled infection (serum hepatitis B virus DNA that is below the limit of detection AND receiving anti-viral therapy for hepatitis B) are permitted upon consultation with the physician managing the infection.
16. Known hypersensitivity to both allopurinol and rasburicase.
17. Pregnant or breast-feeding women.
18. Women of childbearing potential* or men who are unwilling to practice highly effective contraception prior to the initial study drug treatment, during the study, and for at least 6 months after the last dose. Highly effective contraceptive measures include stable use of combined (estrogen and progestogen containing) hormonal contraception (oral, intravaginal, transdermal) or progestogen-only hormonal contraception (oral, injectable, implantable) associated with inhibition of ovulation initiated 2 or more menstrual cycles prior to screening; intrauterine device; intrauterine hormone-releasing system; bilateral tubal ligation; vasectomized partner; and or sexual abstinence†, ‡. * Postmenopausal women must be amenorrheic for at least 12 months in order not to be considered of childbearing potential. Pregnancy testing and contraception are not required for women with documented hysterectomy or tubal ligation.† Sexual abstinence is considered a highly effective method only if defined as refraining from heterosexual intercourse during the entire period of risk associated with the study treatments. The reliability of sexual abstinence needs to be evaluated in relation to the duration of the clinical trial and the preferred and usual lifestyle of the patient.‡ Periodic abstinence (calendar, symptothermal and post-ovulation methods), withdrawal (coitus interruptus), spermicides only, and lactational amenorrhea method (LAM) are not acceptable methods of contraception. Female condom and male condom should not be used together.
19. Administration of live vaccination within 28 days of first administration of study drug.

TABLE 5
CRS Toxicity Grading

| CRS | | | | |
|---|---|---|---|---|
| Parameter | Grade 1 | Grade 2 | Grade 3 | Grade 4 |
| Fever¹ | Temperature ≥ 38° C. | Temperature ≥ 38° C. | Temperature ≥ 38° C. | Temperature ≥ 38° C. |

With

| | | | | |
|---|---|---|---|---|
| Hypotension | None | Not requiring | Requiring a vasopressor | Requiring multiple |
| | | vasopressors | with or without | vasopressors |
| | | | vasopressin | (excluding vasopressin) |

And/or²

| | | | | |
|---|---|---|---|---|
| Hypoxia | None | Requiring low-flow | Requiring high-flow nasal | Requiring positive |
| | | nasal cannula³ or | cannula³, facemask, | pressure (eg CPAP, |
| | | blow-by | nonrebreather mask, or | BiPAP, intubation and |
| | | | Venturi mask | mechanical ventilation) |

CPAP = continuous positive airway pressure, bilevel positive airway pressure, BiPAP = bilevel positive airway pressure.
Organ toxicities associated with CRS may be graded according to CTCAE v4.03 but they do not influence CRS grading.
¹Fever is defined as temperature ≥ 38° C. not attributable to any other cause. In patients who have CRS then receive antipyretic or anti-cytokine therapy such as tocilizumab or steroids, fever is no longer required to grade subsequent CRS severity. In this case, CRS grading is driven by hypotension and/or hypoxia.
²CRS grade is determined by the more severe event: hypotension or hypoxia not attributable to any other cause. For example, a patient with temperature of 39.5° C., hypotension requiring 1 vasopressor, and hypoxia requiring low-flow nasal cannula is classified as grade 3 CRS.
³Low-flow nasal cannula is defined as oxygen delivered at ≤6 L/minute. Low flow also includes blow-by oxygen delivery, sometimes used in pediatrics. High-flow nasal cannula is defined as oxygen delivered at >6 L/minute.

### Example 2: Clinical Evaluation of Anti-CD3 x Anti-CD20 Bispecific Antibody in Patients with Relapsed or Refractory B-Cell Non-Hodgkin Lymphoma

This is a phase 2, open-label, multi-cohort, multi-center study of odronextamab (anti-CD3 x anti-CD20) administered as an IV infusion in patients with B-NHL that have relapsed after or are refractory to prior systemic therapy. The study consists of 5 disease-specific cohorts, each with independent parallel enrollment. Patients in the DLBCL cohort were randomized 1:1 to either Arm 1 or Arm 2 in the initial step of this cohort with 2 different odronextamab dose regimens. Cohort assignments were based on the patient's diagnosis and treatment history at the time of study enrollment.

The treatment period comprised 12 weekly doses followed by every 2 weeks (Q2W) dosing until the time of disease progression or other protocol-defined reason for treatment discontinuation. In addition, if a patient demonstrated a CR and showed a durable response for at least 9 months after the initial determination of CR, then the frequency of study drug administration at the assigned dose was decreased from Q2W to once every 4 weeks (Q4W) intervals, based on local investigator evaluation. Patients must have received the assigned QW full (nominal) dose at the Q2W dosing schedule for at least 3 preceding doses before switching from Q2W to Q4W dosing.

Odronextamab was administered as a single agent intravenously (IV) at an initial split dose of 0.7 mg (0.2/0.5 mg), followed by an intermediate split dose-1 of 4 mg and then an intermediate split dose-2 of 20 mg. Subsequently, dosing in each disease-specific cohort was as follows:
FL grade 1-3a cohort: 80 mg QW followed by 160 mg Q2W
DLBCL cohort Arm 1: 160 mg QW followed by 320 mg Q2W
DLBCL cohort Arm 2: 320 mg QW followed by 320 mg Q2W
MCL after BTK inhibitor therapy: 160 mg QW followed by 320 mg Q2W
MZL: 80 mg QW followed by 160 mg Q2W
Other B-NHL: 160 mg QW followed by 320 mg Q2W

Patients in the DLBCL cohort were randomized 1:1 to either Arm 1 or Arm 2 in the initial step of this cohort with 2 different odronextamab dose regimens. Patients in Arm 1 received a QW full dose of 160 mg during QW dosing followed by 320 mg during Q2W dosing. Patients in Arm 2 received a QW full dose of 320 mg during QW dosing followed by 320 mg during Q2W dosing.

The study population for each of the 5 separate independent enrolling disease-specific cohorts consisted of:
FL grade 1-3a cohort: patients with FL that have relapsed or are refractory to at least 2 prior lines of systemic therapy, including an anti-CD20 antibody and an alkylating agent; patients must have failed combination lenalidomide and rituximab treatment where approved or deemed not appropriate to receive this treatment according to the investigator.
DLBCL cohort: patients with DLBCL that have relapsed or are refractory to at least 2 prior lines of systemic therapy, including an anti-CD20 antibody and an alkylating agent. Patients with de novo DLBCL or DLBCL that is transformed from a lower grade neoplasm (eg, FL or CLL) may be enrolled. Patients with DLBCL transformation from prior CLL can only be enrolled in the absence of a leukemic CLL component. For patients with transformed DLBCL, prior systemic therapies administered for the lower grade neoplasm will not be considered among the prior lines of therapy for the purpose of determining eligibility.
MZL cohort: patients with MZL that have relapsed or are refractory to at least 2 prior lines of systemic therapy.
Other B-NHL cohort: patients with other B-NHL subtypes (excluding WM, SLL, CLL, Burkitt lymphoma and Burkitt-like lymphoma with 11q aberration) that have relapsed or are refractory to at least 2 prior lines of systemic therapy.
MCL after BTK inhibitor therapy cohort: patients with MCL that have failed prior Bruton tyrosine kinase (BTK) inhibitor therapy I.

The initial dose (0.7 mg [0.2/0.5 mg]), intermediate dose-1 of 4 mg, and intermediate dose-2 of 20 mg were always administered as a split infusion over 2 days, preferably consecutive but no more than 3 days apart, even if these doses were delayed beyond treatment week 3. Administration of the QW full (nominal) dose proceeded if the full initial, full intermediate dose-1 and intermediate dose-2 were received and tolerated.

In patients who did not experience grade 3 cytokine release syndrome (CRS) with the initial dose, intermediate dose-1 and intermediate dose-2, the full QW dose of odronextamab was administered as single infusion. Patients received the full initial dose prior to the intermediate dose-1; patients received the full intermediate dose-1 prior to the intermediate dose-2 and received the full intermediate dose-2 prior to receiving the full single infusion. Doses beyond treatment week 4, odronextamab was administered as a single infusion.

When administration of the first full QW dose was tolerated as a single infusion, subsequent doses were administered as a single infusion over 1 to 4 hours depending on previous tolerability.

The treatment duration comprised 12 QW doses followed by Q2W dosing until the time of disease progression or other protocol-defined reason for treatment discontinuation. In addition, if a patient demonstrated a CR and showed a durable response for at least 9 months after the initial determination of CR, then the frequency of study drug administration at the assigned dose was decreased from Q2W to Q4W intervals, based on local investigator evaluation. Patients must have received the assigned full QW dose at the Q2W dosing schedule for at least 3 preceding doses before switching from Q2W to Q4W dosing. Patients were followed for efficacy until the time of disease progression or start of non-protocol anti-lymphoma therapy.

Inclusion Criteria

Each patient must meet the following criteria to be eligible for inclusion in the study:
1. Age 18 years or greater
2. For the FL grade 1-3a cohort only: Central histopathologic confirmation of the FL grade 1 to 3a diagnosis must be obtained before study enrollment. Patients with FL grade 3b are ineligible for this cohort but may be included in the “Other B-NHL” cohort. Follicular lymphoma subtyping is based on the World Health Organization (WHO) classification.
3. Disease-specific cohorts:
   Patients should, in the judgment of the investigator, require systemic therapy for lymphoma at the time of study enrollment and should be deemed not appropriate for any other approved therapy with established benefit for that indication. Refractory is defined as no response (SD/PD) or relapse within months of last treatment.
      FL grade 1-3a cohort: patients with FL grade 1-3a that has relapsed after or is refractory to at least 2 prior lines of systemic therapy, including an anti-CD20 antibody and an alkylating agent; patients must have failed combination lenalidomide and rituximab treatment where approved or deemed not appropriate to receive this treatment according to the investigator.
      DLBCL cohort: patients with DLBCL that has relapsed after or is refractory to at least 2 prior lines of systemic therapy, including an anti-CD20 antibody and an alkylating agent. Patients with de novo DLBCL or DLBCL that is transformed from a lower grade neoplasm (eg, FL or CLL) may be enrolled. Patients with DLBCL transformation from prior CLL can only be enrolled in the absence of a leukemic CLL component. For patients with transformed DLBCL, prior systemic therapies administered for the lower grade neoplasm will not be considered among the prior lines of therapy for the purpose of determining eligibility.
      The following subtypes based on the WHO classification are eligible:
         DLBCL not otherwise specified (NOS)
         Germinal center B-cell type
         Activated B-cell type
      MCL after BTK inhibitor therapy cohort: patients with MCL that have failed prior Bruton tyrosine kinase (BTK) inhibitor therapy.
      MZL cohort: patients with MZL that has relapsed or is refractory to at least 2 prior lines of systemic therapy.
      The following subtypes based on the WHO classification are eligible:
         Extranodal MZL of mucosa-associated lymphoid tissue (MALT lymphoma)
         Nodal marginal zone lymphoma
         Splenic marginal zone lymphoma
      Other B-NHL cohort: patients with B-NHL other than FL grade 1-3a, DLBCL, MCL, or MZL) that has relapsed or is refractory to at least 2 prior lines of systemic therapy. The following subtypes based on the WHO classification are eligible:
         Primary mediastinal (thymic) large B-cell lymphoma
         T-cell/histiocyte-rich large B-cell lymphoma
         Epstein-Barr virus (EBV)+ DLBCL, NOS
         High-grade B-cell lymphoma, with MYC and BCL2 and/or BCL6 rearrangements
         High-grade B-cell lymphoma, NOS
         B-cell lymphoma, unclassifiable, with features intermediate between DLBCL and classical Hodgkin lymphoma
         Follicular lymphoma, grade 3b
      Patients with Waldenström macroglobulinemia (WM, lymphoplasmacytic lymphoma), small lymphocytic leukemia (SLL) and chronic lymphocytic leukemia (CLL), Burkitt lymphoma and Burkitt-like lymphoma with 11q aberration are excluded.
4. Measurable disease on cross sectional imaging (defined as at least 1 bi-dimensionally measurable nodal lesion of 1.5 cm in the greatest transverse diameter (GTD) regardless of the short axis diameter) documented by diagnostic imaging (computed tomography [CT], or magnetic resonance imaging [MRI]).
5. Eastern Cooperative Oncology Group (ECOG) performance status 0 or 1.
6. Adequate bone marrow function as documented by:
   a. Platelet count ≥50×10⁹/L. A patient may not have received platelet transfusion therapy within 7 days prior to first dose of odronextamab in order to meet the platelet eligibility criterion.
   b. Hemoglobin ≥9.0 g/dL
   c. Absolute neutrophil count (ANC)≥1.0×10⁹/L. A patient may not have received granulocyte colony stimulating factor within 2 days prior to first dose of odronextamab in order to meet the ANC eligibility criterion.
      Patients with bone marrow involvement or splenic sequestration should meet the following hematologic parameters:
         Platelet count ≥25×10⁹/L. A patient may not have received platelet transfusion therapy within 3 days prior to first dose of odronextamab in order to meet the platelet eligibility criterion.
         Hemoglobin ≥7.0 g/dL
         Absolute neutrophil count (ANC)≥0.5×10⁹/L. A patient may not have received granulocyte colony stimulating factor within 2 days prior to first dose of odronextamab in order to meet the ANC eligibility criterion.
7. Adequate hepatic function:
   a. Total bilirubin ≤1.5×upper limit of normal (ULN) (≤3×ULN if attributed to lymphoma infiltration of liver)
   b. Alanine aminotransferase (ALT) and aspartate aminotransferase (AST)≤2.5×ULN (≤5×ULN if attributed to lymphoma infiltration of liver)
   c. Alkaline phosphatase (ALP)≤2.5×ULN (≤5×ULN if attributed to lymphoma infiltration of liver)
   NOTES:
      Irrespective of the presence of lymphoma infiltration of the liver, a patient with an AST>2.5×ULN and/or ALT>2.5×ULN concurrent with a total bilirubin >1.5×ULN will be excluded.
      Patients with known Gilbert syndrome will be excluded if the total bilirubin value is >4×upper limit of normal (ULN) for the local general population.
8. Serum creatinine ≤1.5×ULN, or calculated creatinine clearance by Cockcroft-Gault formula ≥50 mL/min.
   NOTE: Patients with a calculated creatinine clearance <50 mL/min may be considered for enrollment if a measured creatinine clearance (based on 24-hour urine collection or other reliable method) is ≥50 mL/min.
9. Willingness to undergo tumor biopsy at baseline. If an investigator has determined that a baseline tumor biopsy cannot be obtained safely, the sponsor may grant an exception to the requirement for biopsy only after discussion with and approval by the medical monitor.
10. Ability to understand the purpose and risks of the study and provide signed and dated informed consent and authorization to use protected health information (in accordance with national and local subject privacy regulations).
11. Willing and able to comply with clinic visits and study-related procedures
12. Provide informed consent signed by study patient or legally acceptable representative
13. Able to understand and complete study-related questionnaires
Exclusion Criteria

A patient who meets any of the following criteria was/will be excluded from the study:
1. Primary central nervous system (CNS) lymphoma or known involvement by non-primary CNS NHL (suspected CNS lymphoma should be evaluated by lumbar puncture, as appropriate, in addition to the mandatory head CT or MRI).
2. Treatment with any systemic anti-lymphoma therapy within 5 half-lives or within 28 days prior to first administration of study drug, whichever is shorter.
3. History of allogeneic stem cell transplantation.
4. Prior treatment with any chimeric antigen receptor T-cell (CAR-T) therapy.
5. Continuous systemic corticosteroid treatment with more than 10 mg per day of prednisone or anti-inflammatory equivalent within 72 hours of start of study drug.
6. History of neurodegenerative condition or CNS movement disorder. Patients with a history seizure within 12 months prior to study enrollment are excluded.
7. Vaccination within 28 days prior to first study drug administration with a vector that has replicative potential.
8. Another malignancy except B-NHL in the past 5 years, with the exception of non-melanoma skin cancer that has undergone potentially curative therapy or in situ cervical carcinoma, or any other tumor that has been deemed to be effectively treated with definitive local control and with curative intent.
9. Evidence of significant concurrent disease or medical condition that could interfere with the conduct of the study or put the patient at significant risk, including but not limited to significant cardiovascular disease (eg, New York Heart Association Class III or IV cardiac disease, myocardial infarction within the previous 6 months, unstable arrhythmias, or unstable angina) and/or significant pulmonary disease (eg, obstructive pulmonary disease and history of symptomatic bronchospasm).
10. Cardiac ejection fraction <40% by echocardiogram or multigated acquisition (MUGA) scan.
11. Any infection requiring hospitalization or treatment with IV anti-infectives within 2 weeks of first administration of study drug.
12. Uncontrolled infection with human immunodeficiency virus (HIV), hepatitis B or hepatitis C infection; or other uncontrolled infection.
   a. Patients with HIV who have controlled infection (undetectable viral load and CD4 count above 350 cells/microliter either spontaneously or on a stable antiviral regimen) are permitted.
   b. Patients with hepatitis B (HepBsAg+) who have controlled infection (serum hepatitis B virus DNA polymerase chain reaction [PCR] that is below the limit of detection AND receiving anti-viral therapy for hepatitis B) are permitted.
   c. Patients who are hepatitis C virus antibody positive (HCV Ab+) who have controlled infection (undetectable HCV RNA by PCR either spontaneously or in response to a successful prior course of anti-HCV therapy) are permitted.
13. History of severe allergic reaction attributed to compounds with a similar chemical or biologic composition as that of the study drug or excipient. A severe allergic reaction is defined for this purpose as that requiring hospitalization and/or treatment with epinephrine.
14. Known hypersensitivity to both allopurinol and rasburicase
15. Women of childbearing potential (WOCBP) with a positive serum β-hCG pregnancy test are ineligible for this study.
16. Pregnant or breastfeeding women.
17. Women of childbearing potential* or men who are unwilling to practice highly effective contraception prior to the initial dose/start of the first treatment, during the study, and for at least 6 months after the last dose. Sperm donation is prohibited during the study and for 6 months after the last dose of study drug. Highly effective contraceptive measures include:
   a. stable use of combined (estrogen and progestogen containing) hormonal contraception (oral, intravaginal, transdermal) or progestogen-only hormonal contraception (oral, injectable, implantable) associated with inhibition of ovulation initiated 2 or more menstrual cycles prior to screening
   b. intrauterine device (IUD); intrauterine hormone-releasing system (IUS)
   c. bilateral tubal ligation
   d. vasectomized partner (provided that the male vasectomized partner is the sole sexual partner of the study participant and that the partner has obtained medical assessment of surgical success for the procedure).
   e. and/or sexual abstinences†, ‡. * Women of childbearing potential are defined as women who are fertile following menarche until becoming post-menopausal, unless permanently sterile. Permanent sterilization methods include hysterectomy, bilateral salpingectomy, and bilateral oophorectomy.A post-menopausal state is defined as no menses for 12 months without an alternative medical cause. A high follicle stimulating hormone (FSH) level in the postmenopausal range may be used to confirm a post-menopausal state in women not using hormonal contraception or hormonal replacement therapy. However, in the absence of 12 months of amenorrhea, a single FSH measurement is insufficient to determine the occurrence of a post-menopausal state. The above definitions are according to Clinical Trial Facilitation Group (CTFG) guidance.Pregnancy testing and contraception are not required for women with documented hysterectomy or tubal ligation.† Sexual abstinence is considered a highly effective method only if defined as refraining from heterosexual intercourse during the entire period of risk associated with the study drugs. The reliability of sexual abstinence needs to be evaluated in relation to the duration of the clinical trial and the preferred and usual lifestyle of the patient.‡ Periodic abstinence (calendar, symptothermal, post-ovulation methods), withdrawal (coitus interruptus), spermicides only, and lactational amenorrhea method (LAM) are not acceptable methods of contraception. Female condom and male condom should not be used together.
18. Prior treatment with an anti-CD20 x anti-CD3 bispecific therapy.

TABLE 6
CRS Toxicity Grading

| CRS | | | | |
|---|---|---|---|---|
| Parameter | Grade 1 | Grade 2 | Grade 3 | Grade 4 |
| Fever¹ | Temperature ≥ 38° C. | Temperature ≥ 38° C. | Temperature ≥ 38° C. | Temperature ≥ 38° C. |

With

| | | | | |
|---|---|---|---|---|
| Hypotension | None | Not requiring | Requiring a vasopressor | Requiring multiple |
| | | vasopressors | with or without | vasopressors |
| | | | vasopressin | (excluding vasopressin) |

And/or²

| | | | | |
|---|---|---|---|---|
| Hypoxia | None | Requiring low-flow | Requiring high-flow nasal | Requiring positive |
| | | nasal cannula³ or | cannula³, facemask, | pressure (eg CPAP, |
| | | blow-by | nonrebreather mask, or | BiPAP, intubation and |
| | | | Venturi mask | mechanical ventilation) |

CPAP = continuous positive airway pressure, bilevel positive airway pressure, BiPAP = bilevel positive airway pressure.
Organ toxicities associated with CRS may be graded according to CTCAE v4.03 but they do not influence CRS grading.
¹Fever is defined as temperature ≥ 38° C. not attributable to any other cause. In patients who have CRS then receive antipyretic or anti-cytokine therapy such as tocilizumab or steroids, fever is no longer required to grade subsequent CRS severity. In this case, CRS grading is driven by hypotension and/or hypoxia.
²CRS grade is determined by the more severe event: hypotension or hypoxia not attributable to any other cause. For example, a patient with temperature of 39.5° C., hypotension requiring 1 vasopressor, and hypoxia requiring low-flow nasal cannula is classified as grade 3 CRS.
³ Low-flow nasal cannula is defined as oxygen delivered at ≤6 L/minute. Low flow also includes blow-by oxygen delivery, sometimes used in pediatrics. High-flow nasal cannula is defined as oxygen delivered at >6 L/minute.

Results for Follicular Lymphoma

As discussed herein, odronextamab is a hinge-stabilized, human IgG4-based CD20xCD3 bispecific antibody that binds CD20 on B cells and CD3 on T cells, triggering T-cell-mediated cytotoxicity of malignant B-cells. In a phase I study (discussed in Example 4), patients with FL grade 1-3a receiving prior lines of therapy and treated with odronextamab doses of mg, had an ORR of 91% and CR rate of 72%. Responses were durable with a 4 y progression-free survival (PFS) rate of 54%. The phase II dose in R/R FL patients was determined as 80 mg weekly. Below are results from a pre-specified analysis of the FL grade 1-3a cohort from the phase II study, which incorporated a step-up regimen designed to maintain efficacy while minimizing acute toxicity including cytokine release syndrome (CRS).

In the phase II study, adult patients with FL grade 1-3a who had relapsed or were refractory to prior lines of therapy including an anti-CD20 antibody and alkylator were enrolled. IV odronextamab was administered in 21d cycles with steroid prophylaxis and weekly step-up dosing during Cycle 1 (C1) to mitigate risk of acute toxicity. The initial step-up regimen consisted of 1 mg split over C1 Day 1 (D1) and C1D2, and 20 mg split over C1D8 and C1D9, followed by the 80 mg full dose on C1D15 ( 1/20 regimen). The 1/20 regimen was revised during the study to further mitigate CRS risk by adding an intermediary step-up dose. The modified regimen consisted of 0.7 mg split over C1D1 (0.2 mg) and C1D2 (0.5 mg), 4 mg split over C1D8 and C1D9, and 20 mg split over C1D15 and C1D16, followed by the 80 mg full dose on C2D1 (0.7/4/20 regimen). 80 mg weekly continued until the end of C4. After C4, maintenance treatment with 160 mg odronextamab occurred every 2 weeks until disease progression or unacceptable toxicity. The primary endpoint was ORR assessed by independent central review (ICR) according to Lugano 2014 criteria. CRS was assessed using 2019 ASTCT criteria.

As of Apr. 20, 2022, 96 patients were evaluable for safety; 85 for efficacy. Median age 59 years (range 22-84), 52% male, 58% FLIPI 3-5, 15.6% had bulky disease, median prior lines of therapy 3 (range 2-13). 74% were refractory to their last therapy, 79% refractory to prior anti-CD20 therapy and 48% had progression of disease within 2 y (POD24). Median duration of study follow-up was 17.3 months. ORR and CR rate confirmed by ICR were 81% ( 69/85) and 75% (64/85), respectively. ORR and CR rate were consistent across high-risk subgroups, including patients aged 65 years, POD24, FLIPI 3-5 and patients refractory to their last line of therapy; ORR and CR rate were also consistent for the subgroup of patients treated with the 0.7/4/20 step-up regimen. Responses were durable with both a median duration of response and a median duration of CR of 18.2 months. Median PFS was 20.2 mos and median OS was not reached (95% CI 23.0 mos—not estimable).

TEAEs occurred in 95 (99%) patients, considered treatment related in 86 (90%). In the overall safety evaluable population, the most common TEAEs (>30% all grades) were CRS (51%), pyrexia (32%), anemia (31%), and infusion-related reaction (31%). Following implementation of the 0.7/4/20 step-up regimen in C1, grade 1 CRS was observed in 39% of patients; no grade ≥2 CRS was reported. All CRS events resolved and only 1 patient received tocilizumab for CRS management. No ICANS were reported following revisions to step-up dosing compared with 3% in the 1/20 regimen. Treatment-related grade 5 AEs were reported for 2 (2%) patients; treatment-related AEs led to discontinuation in 6 (6%) patients.

Consistent with the phase I study, in the phase II study odronextamab demonstrated compelling efficacy in patients with FL grade 1-3a receiving prior lines of therapy, with 75% of patients achieving CR confirmed by ICR. Durability of responses and favorable survival outcomes are clinically important in the context of heavily pretreated, highly R/R FL, where prognosis is typically poor. The overall tolerability profile with the 0.7/4/20 step-up regimen was favorable, with only grade 1 CRS observed during C1.

Results for Diffuse Large B-Cell Lymphoma

As discussed herein, odronextamab is a hinge-stabilized, human IgG4-based CD20xCD3 bispecific antibody that binds CD20 on B cells and CD3 on T cells and, triggering T-cell-mediated cytotoxicity of malignant B-cells. In a phase I study (discussed in Example 4) odronextamab demonstrated encouraging activity in patients with DLBCL receiving prior lines of therapy. DLBCL patients treated with odronextamab at doses of ≥80 mg had an ORR of 53% and a CR rate of 53%. Responses were durable with 88% probability of an ongoing response at 12 months. The phase II dose for expansion in R/R DLBCL patients was determined as 160 mg weekly. Below are results from a pre-specified analysis of the 160 mg DLBCL cohort from the phase II study, which incorporated a step-up regimen designed to maintain efficacy while minimizing acute toxicity including cytokine release syndrome (CRS).

In the phase II study, adult patients with DLBCL who had relapsed or were refractory to prior lines of therapy including an anti-CD20 antibody and an alkylator were enrolled. IV odronextamab was administered in 21-day cycles with steroid prophylaxis and weekly step-up dosing during Cycle 1 (C1) to mitigate risk of acute toxicity. The initial step-up regimen consisted of 1 mg split over C1 Day 1 (D1) and C1D2, and 20 mg split over C1D8 and C1D9, followed by the 160 mg full dose on C1D15 ( 1/20 regimen). The 1/20 regimen was revised during the study to further mitigate CRS risk by adding an intermediary step-up dose. The modified regimen consisted of 0.7 mg split over C1D1 (0.2 mg) and C1D2 (0.5 mg), 4 mg split over C1D8 and C1D9, and 20 mg split over C1D15 and C1D16, followed by the 160 mg full dose on C2D1 (0.7/4/20 regimen). 160 mg weekly continued until the end of C4. After C4, maintenance treatment was 320 mg odronextamab every 2 weeks until disease progression or unacceptable toxicity. The primary endpoint was ORR assessed by independent central review (ICR) according to Lugano 2014 criteria. CRS was assessed using 2019 ASTCT criteria.

As of Apr. 20, 2022, 121 patients with DLBCL were evaluable for safety; 90 for efficacy. Median age 67 years (range 24-88), male 60%, Ann Arbor stage III-IV 80%, IPI score 58%, median prior lines of therapy 2 (range 2-8). 56% were primary refractory and 65% were double refractory. Median duration of study follow-up was 17.1 months. ORR and CR rate confirmed by ICR were 53% (48/90) and 37% (33/90), respectively. ORR and CR rates were consistent across high-risk subgroups and in the subgroup of patients treated with the 0.7/4/20-step-up regimen. CRs were durable; median duration of CR was not reached (95% CI: 10.2 months—not estimable) and the probability of an ongoing CR at 9 months was 70%.

TEAEs occurred in 117 (97%) patients, considered treatment related in 102 (84%). In the overall safety-evaluable population, the most common TEAEs (>30% all grades) were CRS (53%), pyrexia (41%), and anemia (34%). Following implementation of the 0.7/4/20 step-up regimen in C1, no grade ≥3 CRS events were observed. Only grades 1 and 2 CRS were observed in 35% and 13% of DLBCL patients, respectively. All CRS events resolved with supportive measures; 20% of pts received tocilizumab and none required vasopressors or mechanical ventilation for CRS management. ICANS were reported in only 2 pts (3%) following revisions to step-up dosing, and both were low grade, ICANS occurred in 6% pts with the 1/20 regimen. Treatment-related grade 5 AEs occurred in 2 patients (2%), and treatment-related AEs led to odronextamab discontinuation in 8 patients (7%).

In the phase II study, odronextamab demonstrated clinically meaningful efficacy, durable CRs, and favorable safety in a hard-to-treat, highly aggressive patient population with R/R DLBCL. The results of this phase 2 study confirm the activity observed in phase I, which collectively demonstrate that odronextamab has compelling activity both before and after CAR-T therapy and a tolerable safety profile.

The 0.7/4/20 odronextamab step-up dose regimen for C1 mitigates the risk for high grade CRS, which has been observed consistently with other bispecifics and CAR-T therapies. Additionally, with the 0.7/4/20 regimen only low grade ICANS have been reported.

### Example 3: Premedications Applied to Odronextamab IV Administration

The following premedications will be applied to odronextamab administration from the initial dose through the first once-a-week (QW) dose as a single infusion, as discussed in the Examples herein.

If the patient experiences an infusion-related reaction (IRR) and/or cytokine release syndrome (CRS) of any grade with first QW dose, premedications will be continued until the full QW dose is tolerated without IRR or CRS:
1. 12 to 24 hours prior to planned start time of first split infusion and prior to planned start of second split infusion if administered on non-consecutive days for each odronextamab dose:
   a. Dexamethasone 20 mg PO or equivalent dose of steroid;
2. Premedication on each day of split infusion of odronextamab and on the day of full QW dose as a single infusion:
   a. Dexamethasone 20 mg IV 1 to 3 hours prior to start of infusion on the day of treatment
   b. Diphenhydramine 25 mg IV or PO 30 to 60 min before (can be replaced with another equivalent antihistamine)
   c. Acetaminophen 650 mg PO 30 to 60 min before, unless the patient has received it within the past 4 hours prior to infusion with odronextamab, or is allergic to acetaminophen
3. 24 (±4) hours from the end of second split infusion or end of the first full QW dose as a single infusion:
   a. Dexamethasone 20 mg PO or equivalent dose of steroid

First dose administration following full QW dose administered as a single infusion without experiencing an IRR and/or CRS of any grade with 20 mg of dexamethasone IV:
a. Dexamethasone 10 mg IV 1 to 3 hours prior to start of infusion on the day of treatment
b. Diphenhydramine 25 mg IV or PO 30 to 60 min before (can be replaced with another equivalent antihistamine)
c. Acetaminophen 650 mg PO 30 to 60 min before, unless the patient has received it within the past 4 hours prior to infusion with odronextamab, or is allergic to acetaminophen

For subsequent doses no premedication is required if the single infusion is tolerated without experiencing an IRR and/or CRS of any grade with the reduced 10 mg of dexamethasone IV.

Anti-IL6 therapy (e.g., tocilizumab) is administered 8 mg/kg infused over 1 hour, dose not to exceed 800 mg

### Example 4: Odronextamab, a Human CD20xCD3 Bispecific Antibody, in Relapsed or Refractory B-Cell Non-Hodgkin Lymphoma

Odronextamab is a hinge-stabilized, fully human IgG4-based CD20xCD3 bispecific antibody that binds CD3+ T cells and malignant B cells. Following are the findings from a first-in-human study of odronextamab in patients with relapsed/refractory (R/R) B-cell non-Hodgkin lymphoma (B-NHL).

This phase 1 study enrolled patients with CD20-positive relapsed/refractory (R/R) B-cell malignancies previously treated with CD20-directed antibody therapy. Eligible patients, aged ≥18 years, and with an ECOG performance status of received odronextamab intravenously, consisting of step-up dosing in cycle 1, followed by weekly treatment at a target dose level ranging from 0.1-320 mg during cycles 2-4. Maintenance treatment was every 2 weeks until progression. Primary endpoints were safety, pharmacokinetics, and the maximum tolerated dose, as well as preliminary anti-tumor activity.

A total of 145 patients were enrolled (dose escalation, n=94; dose expansion, n=51). Patients received a median of three prior lines of therapy (range: 1-11), 42 (29.0%) received prior chimeric antigen receptor (CAR) T cell therapy, and 119 (82.1%) were refractory to the last line of therapy. The median duration of follow-up was 4.2 months (interquartile range 1.6-11.5). During dose escalation, odronextamab was administered up to the maximum dose of 320 mg weekly with no dose-limiting toxicities observed. A recommended dose of odronextamab 80 mg was selected for dose-expansion in patients with R/R follicular lymphoma (FL) grade 1-3a, and odronextamab 160 mg was selected for expansion in patients with R/R diffuse large B-cell lymphoma (DLBCL). The most frequent adverse events were pyrexia, cytokine release syndrome (CRS), chills, anaemia and fatigue. CRS and neurotoxicity were predominantly low grade and did not result in treatment discontinuation.

The objective response rate (ORR) for patients with all B-NHL subtypes treated with odronextamab across all doses was 51%, and 37% had a complete response. In patients with FL 1-3a treated with odronextamab doses ≥5 mg (n=32), the ORR was 91%, and 72% had a complete response. In DLBCL patients without prior CAR T treated with odronextamab doses ≥80 mg (n=15), the ORR was 53%, of which all were complete responses. In DLBCL patients with prior CAR T treated with odronextamab doses ≥80 mg (n=30), the ORR was 33%, and 27% had a complete response. Responses appeared durable with 60%, 88% and 100% of complete responses ongoing at 12 months in patients with FL 1-3a, DLBCL without CAR T, and DLBCL following prior CAR T, respectively.

Study Design and Treatment

Patients were eligible for enrolment if they were aged ≥18 years with documented B-NHL, had at least one measurable lesion, prior treatment with an anti-CD20 antibody, an Eastern Cooperative Oncology Group Performance Status (ECOG PS) of 0 or 1, and had adequate haematologic and organ function. Patients with primary central nervous system (CNS) lymphoma, or prior allogeneic stem cell transplantation were excluded.

A traditional 3+3 dose-escalation design was implemented during dose escalation. Patients received odronextamab intravenously according to a step-up dosing schedule in cycle 1, followed by weekly treatment at a target dose level ranging from 0.1-320 mg during cycles 2-4 (each cycle was 21 days). Maintenance treatment was every 2 weeks until disease progression or unacceptable toxicity. Prophylactic measures to mitigate the risk for cytokine release syndrome (CRS), including steroid prophylaxis, split dosing and step-up dosing were implemented during the course of dose escalation.

On-site availability of tocilizumab was confirmed prior to step-up dosing. Odronextamab was administered intravenously over 4 hours during cycle 1 (days 1, 2, 8, 9, 15, 16), with the first full dose administered on day 1 of cycle 2. The infusion duration was decreased to 60 minutes for subsequent doses during cycles 2-4 and during maintenance treatment. Patients were monitored in the inpatient setting for approximately 24 hours beyond the end of each infusion during cycle 1 and day 1 of cycle 2. Subsequent doses were administered in the outpatient setting. Premedications were administered to further mitigate the risk for CRS during cycle 1; full details are provided in Example 3. In brief, prophylactic steroids were given 1 day prior to, on the day of, and 1 day after the infusion. In addition, acetaminophen and diphenhydramine were given 30-60 min before the infusion. Steroid premedication was tapered for the day 8 dose in cycle 2 and all premedications were discontinued for subsequent doses. Treatment at the target dose level occurred on days 1, 8 and 15 of cycles 2-4, and was followed by maintenance dosing every 2 weeks thereafter.

Patients were permitted to receive supportive care medications as needed for the management of CRS (including antipyretics, corticosteroids, intravenous fluids, vasopressors, tocilizumab, oxygen and mechanical ventilation), hypouricemic agents for management of tumour-lysis syndrome (TLS), and anti-infective prophylaxis as per local institutional guidelines, including Pneumocystis jirovecii pneumonia prophylaxis, IVIG supplementation, and appropriate antiviral prophylaxis for patients with prior herpes simplex virus, cytomegalovirus or hepatitis B virus infections. Transfusion of blood products and granulocyte colony-stimulating factor use was permitted as per standard of care.

Study End Points and Assessments

The primary objective was to assess safety, tolerability, and dose-limiting toxicities (DLTs). Secondary objectives were to evaluate antitumour activity, pharmacokinetics (PK), and immunogenicity.

The primary endpoint was safety, assessed by the incidence of adverse events (AEs) and DLTs, to determine the maximum tolerated dose (MTD) and/or phase 2 dose of odronextamab. Key secondary endpoints included pharmacokinetics, immunogeniticyt, and anti-tumour activity as measured by objection response rate (ORR), duration of response (DoR), and progression-free survival (PFS). The treatment-emergent period was defined as time from first administration of study drug to 90 days after last dose of study drug. Relative dose intensity was defined as actual dose intensity (administered dose per unit of time) divided by the planned dose intensity. The severity of AEs was graded using the National Cancer Institute-Common Terminology Criteria for Adverse Events Version 4.03, and CRS was graded according to criteria, depending on the patient enrolment date. AEs were deemed related to study drug per investigator assessment.

The antitumour activity of odronextamab was measured as objective response rate (ORR), assessed every 12 weeks according to the revised response criteria for malignant lymphoma of the NCI-International Working Group, using the Lugano classification.

Blood samples for odronextamab concentration measurement were collected with dense PK sampling during weeks 1 to 12 and less dense sampling thereafter. Odronextamab concentrations in serum were measured using a validated enzyme-linked immunosorbent assay with a lower limit of quantification of 0.003 mg/L. Descriptive statistics were used to summarize odronextamab concentrations in serum over time.

A preliminary exposure-response (E-R) analysis with logistic regression was conducted using best overall response data from patients with FL or DLBCL with no prior chimeric antigen receptor T cell (CAR T) treatment following odronextamab IV regimens over a dose range from 2 to 320 mg during dose escalation. This analysis evaluated the relationship between exposure metrics (average concentration [C_{avg}] or area under the curve [AUC] over time, and predose concentration at week 12 [Cₘᵢₙ]), and ORR and complete response (CR) rate, over 12 weeks of treatment.

Statistical Analyses

A sample size of 102 patients with B-NHL for dose escalation was determined, based on the 3+3 design and number of dose levels. A total of 130 patients with B-NHL were planned for inclusion in expansion cohorts to further evaluate the safety and efficacy of recommended doses. No formal statistical hypotheses were implemented in this study. All B-NHL patients from the dose-escalation and dose-expansion portions of the study were pooled for analyses. For analysis of baseline characteristics and safety, all treated patients were included, and data were also evaluated by dose group. Efficacy endpoints were analyzed by B-NHL subtype and dose group in patients who had opportunity for response assessment at 12 weeks. For continuous variables, descriptive statistics were presented, including the number of patients reflected in the calculation (n), mean, median, standard deviation, minimum, and maximum. For categorical or ordinal variables, frequencies and percentages were displayed for each category. For time-to-event variables, medians and 95% confidence intervals (CI) were provided by the Kaplan-Meier method, if applicable. ORR (proportion of patients with best overall response of CR/PR per Lugano classification) and CR rate (proportion of patients with best overall response of CR per Lugano classification) were summarized with the two-sided 95% exact binomial CI using the Clopper-Pearson method. Patients who were not evaluable for best overall response were considered non-responders. Duration of response (time from first CR/PR to disease progression or death), duration of CR (time from first CR to disease progression or death) and PFS (time from start of treatment to disease progression or death) were analyzed by Kaplan-Meier estimation; patients without disease progression or death were censored at the last adequate tumour assessment on or before the data cutoff date.

Results

A total of 145 patients were enrolled; 94 into the dose-escalation portion and 51 into the dose-expansion portion of the study. Of 145 B-NHL patients, 98 (68%) had aggressive B-NHL (DLBCL, n=85; MCL, n=12; FL grade 3b, n=1) and 47 (32%) patients had indolent B-NHL (FL grade 1-3a, n=40; MZL, n=6; Waldenström macroglobulinaemia, n=1). Among the DLBCL patients, 35 were enrolled after failure of prior CD19-directed CAR T therapy.

Patient demographics are summarized as follows: Median age was 67 (interquartile range 57-73) years and the majority of patients (n=101; 70%) were male. At study entry, all patients had an ECOG PS of 0 (n=58; 40%) or 1 (n=87; 60%), 85% (n=123) had Ann Arbor Stage III-IV disease, and 34% (n=49) had bulky disease. The patients comprised a heavily pre-treated and refractory population, receiving a median of three prior lines of systemic therapy (interquartile range 2-5), including autologous stem cell transplant (ASCT; n=12; 8%) and CAR T therapy (n=42; 29%). Overall, 119 (82%) patients were refractory to last line of therapy; 123 (85%) were refractory to an anti-CD20 antibody in any line of therapy, and 100 (69%) were double refractory to an alkylator and an anti-CD20 antibody in any line of therapy.

At data cut-off, seven (5%) patients were continuing to receive treatment (all in the dose-expansion cohort), 33 (23%) completed treatment, and 105 (72%) patients had discontinued treatment, mainly due to progressive disease (n=63, 43%). Median duration of follow-up was 4.2 (interquartile range 1.6-11.5) months. During dose escalation, odronextamab was administered up to the maximum dose of 320 mg weekly; no DLTs were observed, MTD was not reached, and there was no dose-dependent increase in toxicity. Seven patients (5%) underwent re-treatment with odronextamab, of whom three had FL, three had DLBCL, and one had MCL. Six of the re-treated patients received doses <5 mg, and one patient received 160 mg; all cases of re-treatment occurred in patients enrolled in the dose-escalation phase of the study.

Patients received a median of 10 (interquartile range 4-20) doses of odronextamab with a median 13.1 (interquartile range 5.0-32.9) weeks of exposure. The median relative dose intensity was 1.0. Clinical activity was observed across all dose groups, increasing substantially in patients with FL receiving odronextamab ≥5 mg (n=32), and in patients with DLBCL receiving odronextamab ≥80 mg (n=45).

Following IV infusion, peak odronextamab serum concentrations were observed during the end of infusion or 4 hours post-infusion on week 1 day 2 and week 2 day 2 when doses were split. Median serum concentrations of odronextamab generally increased in a dose-dependent manner. Odronextamab showed non-linear PK properties with target-mediated drug disposition. At full doses ranging from 12 mg to 320 mg between week 3 and week 12, the drug accumulation ratio with weekly dosing was up to three-times for median peak concentrations and around two- to six-times for median trough concentrations.

A preliminary exposure-response (E-R) analysis evaluated the relationship between exposure metrics and ORR and CR rate over 12 weeks of treatment using data from 40 FL patients and 38 DLBCL patients without prior CAR T therapy enrolled during dose escalation. Compared with C_{avg} and AUC, Cₘᵢₙ at week 12 provided the best correlation with clinical response in patients with FL or DLBCL without prior CAR T. Positive relationships were observed between probability of ORR or CR and the Cₘᵢₙ, at week 12 in patients with FL and DLBCL and the highest CR rate was observed at doses ≥80 mg.

Immunogenicity against odronextamab was low. Among 137 patients with B-NHL who had available antidrug antibody (ADA) data, one patient who received odronextamab 2 mg had treatment-emergent ADA with very low titre, only at the final visit of safety follow-up (day 30 post-last dose).

All 145 patients experienced at least one treatment emergent adverse event (TEAE) of any grade, and 93% (n=135) of patients experienced at least one TEAE related to treatment. The most frequent TEAEs of any grade were pyrexia (n=107; 74%), CRS (n=89; 61%), chills (n=68; 47%), anaemia (n=55; 38%), and fatigue (n=47; 32%). Grade TEAEs occurred in 119 patients (82%); those occurring in >10% of patients included anaemia (n=36; 25%; treatment-related: n=22; 15%), lymphopenia (n=28; 19%; treatment-related: n=24; 17%), hypophosphataemia (n=27; 19%; treatment-related: n=18; 12%), neutropenia (n=27; 19%; treatment-related: n=21; 14%), and thrombocytopenia (n=20; 14%; treatment-related: n=11; 8%). Febrile neutropenia occurred in four patients (3%; treatment-related: n=3; 2%). In total, 71 (49%) patients experienced infections; 33 (23% of patients had grade ≥3 infections (grade 3, n=28; 19%; grade 4, n=1; 1%; grade 5, n=4; 3%), of which the most frequent was pneumonia (n=12; 8%).

TEAEs of special interest, including infusion-related reactions (IRRs), CRS, CNS/ICANS-like events, tumour lysis syndrome (TLS), and infections, occurred. The majority of CRS events were grade 1 (n=52; 36%) or 2 (n=27; 19%). Grade 3 CRS events occurred in nine (6%) patients, and during dose expansion one MCL patient (1%) had a grade 4 CRS event in the context of grade 5 TLS. CRS was predominantly confined to cycle 1 (step-up dosing) and resolved within a median of 2 days with supportive measures. All grade CRS events occurred prior to the optimization of CRS risk mitigation measures during cycle 1. Tocilizumab was used to manage grade ≥3 CRS in seven (5%) patients, and no patient had to discontinue treatment due to CRS. ICANS-like events were noted in 18 (12%) patients, and four (2.8%) patients had grade 3 events (confusional state, somnolence, encephalopathy, aphasia and cognitive disorder, each n=1). Most of the neurologic TEAEs were transient and resolved within a median of 3 days; none led to treatment discontinuation, and there were no grade 4 or 5 events.

Serious TEAEs occurred in 89 (61%) of 145 patients, most frequently CRS (n=41, 28%), pyrexia (n=11, 8%), pneumonia (n=9, 6%), and IRR (n=6, 4%). Sixty-eight (47%) patients had a serious TEAE in the first 4 weeks of treatment, and in 88 (61%) patients a serious TEAE necessitated prolonged or new hospitalisation.

Of the 145 patients, 70 (48%) patients had a TEAE leading to treatment interruption/delay, and 14 (10%) patients had a TEAE leading to a dose reduction. Twelve (8%) patients discontinued odronextamab due to a TEAE; ten TEAEs were considered treatment-related (grade 1 cytomegalovirus infection, n=1; grade 1 gait disorder, n=1; grade 3 pneumonia, n=2; grade 3 haemolysis, n=1; grade 3 toxoplasmosis, n=1; grade 3 fatigue, n=2; grade 4 elevated AST, n=1; grade 5 TLS, n=1), and three were unrelated to treatment (grade 3 neck abscess in a patient with prior spinal surgery, n=1; grade 3 device-related infection, n=1; leukaemia, n=1). Seventeen patients died on-treatment or within 30 days of the last dose; 12 of these deaths were due to disease progression, and four due to AEs, of which three were considered related to treatment (gastric perforation in a patient with gastric involvement by lymphoma [n=1; time between last dose of study drug and event onset=6 days], lung infection [n=1; 22 days], and TLS [n=1, 3 days]). One fatal event of cardiac arrest was considered unrelated to study drug by the investigator (occurring 22 days after the last dose of study drug). In one patient, the primary cause of death was recurrence/progression of esophagogastric cancer.

There were no additional safety signals among patients who underwent re-treatment with odronextamab (n=7). The most frequent TEAEs among these patients were nausea (n=4), anaemia (n=3) and back pain (n=3). One patient had grade 1 CRS, and four grade TEAEs were reported: hyponatraemia, hypophosphatemia, lymphopenia and intervertebral disc protrusion (all n=1). No serious TEAEs or TEAEs leading to treatment discontinuation were reported in patients undergoing re-treatment.

Overall, across all patients dosed, at all dose levels, no dose-safety relationship was observed.

Antitumour activity was observed across all dose groups (0.03-320 mg) and in all lymphoma subtypes. Of the 145 patients dosed, 142 were evaluable for efficacy with a response assessment at week 12. ORR was 51% (n=72; 95% CI: 42-59), and the CR rate was 37% (n=52; 95% CI: 29-45) for all patients. In FL grade 1-3a patients (n=40), the ORR and CR rate was 78% (n=31; 95% CI: 62-89%) and 63% (n=25; 95% CI: 46-77), respectively. Responses occurred early, with a median time to CR of 2.6 months (interquartile range 2.5-2.9). The median duration of response (DoR) was 12.7 months (95% CI: 6.1, not estimable [NE]; observed range: 1.2-53.0+; interquartile range 4.4-19.9), and the median duration of CR (DoCR) was 14.5 months (95% CI: 8.8, NE; observed range: 0.0+-53.0+; interquartile range 3.9-19.9). In R/R FL patients treated with odronextamab ≥5 mg (n=32), the ORR and CR rate was 91% (n=29; 95% CI: 75-98) and 72% (n=23; 95% CI: 53-86), respectively; median duration of response (DoR) was 15.8 months (95% CI: 6.4, NE; interquartile range 5.2-19.9), with the longest CR still ongoing at 53.0+ months. The estimated probability of maintaining a CR was 60% (95% CI: 34-79) at 12 months, and 54% (95% CI: 28-74) at 48 months. Median progression-free survival (PFS) was 17.1 months (95% CI: 7.5, NE).

In all DLBCL patients without prior CAR T (n=49) treated with odronextamab (0.03-320 mg), the ORR and CR rate was 39% (n=19; 95% CI: 25-54) and 24% (n=12; 95% CI 13-39), respectively. The median time to CR was 2.3 months (interquartile range 1.0-2.8), the median DoR was 4.4 months (95% CI: 2.9, NE; observed range: 1.5-41.4+; interquartile range 2.8-21.0), and the median DoCR was not reached (95% CI: 4.0, NE; observed range: 2.9-34.9+; interquartile range 4.2-21.4). In patients treated with odronextamab ≥80 mg (n=15), which was determined to be the active dose range for aggressive lymphoma, ORR was 53% (n=8; 95% CI: 27-79), and all the responses were complete responses; median DoR was not reached (95% CI: 2.9, NE; interquartile range 4.4-21.2), with the longest CR ongoing at 32.4+ months. The estimated probability of maintaining a CR at 12 and 24 months was 88% (95% CI: 39-98) and 66% (95% CI: 16-91), respectively. Median PFS was 11.5 months (95% CI: 0.5, NE).

Across all DLBCL patients with prior CAR T (n=33) treated with odronextamab (0.03-320 mg), the ORR and CR rate was 33% (n=11; 95% CI: 18-52) and 24% (n=8; 95% CI: 11-42), respectively. The median time to CR was 1.5 months (interquartile range 0.8-2.6), the median DoR was not reached (95% CI: 1.6, NE; observed range: 0.0+-20.5+; interquartile range 1.6-12.8), and nor was the median DoCR (95% CI: NE, NE; observed range: 0.0+-20.5+; interquartile range 2.6-15.8). In patients treated with odronextamab ≥80 mg (n=30), the ORR and CR rate was 33% (n=10; 95% CI: 17-53) and 27% (n=8; 95% CI: 12-46), respectively; median DoR was not reached (95% CI: 1.6, NE; interquartile range 1.6-12.8), with the longest CR ongoing at 20.5+ months. Importantly in this specific group of DLBCL patients treated with odronextamab ≥80 mg, the estimated probability of maintaining a CR at 12 months was 100% (95% CI: NE, NE). Median PFS was 2.0 months (95% CI: 0.9, 2.8).

No patients who were re-treated with odronextamab achieved additional responses.

### DISCUSSION

In this first-in-human study, odronextamab monotherapy demonstrated encouraging anti-tumour activity, robust durability of responses and an overall manageable safety profile in the setting of heavily pre-treated, highly refractory, hard-to-treat patients with B-NHL. Step-up dosing was effective at mitigating the risk of CRS, which occurred predominantly during cycle 1 and resolved within a median of 2 days with supportive care measures. The majority of patients achieved full target dose exposure with minimal treatment delays; no patient experienced DLTs and the MTD was not reached. In patients with R/R FL 1-3a, 80 mg was selected as the recommended dose for further expansion, and 160 mg was selected as the recommended dose for patients with R/R DLBCL. This is the first study to report long-term treatment outcomes for a CD20xCD3 bispecific antibody that includes patients treated during dose-escalation and dose-expansion portions of a trial.

Early phase studies often enroll a diverse and heterogenous pool of patients. Overall, the baseline characteristics of patients enrolled in the present study were consistent with those represented in contemporary trials of CD20xCD3 bispecific antibodies. The patient population included older, heavily pre-treated patients, the majority of whom were double-refractory to both prior alkylator and anti-CD20 therapy. However, one notable difference is that 29% of the patients enrolled in this study had failed prior CAR T therapy at study entry, which is considerably higher than the 2% and 9% rates reported in recent studies of glofitamab and epcoritamab, respectively. As such, the population included in this trial may embody a particularly poor prognosis as these patients will have been exposed both to potential long-term toxicities that are unique to CAR T therapies and their associated preconditioning regimens. The proportion of patients with prior ASCT was 8%, which is numerically lower than some recent studies investigating CD20xCD3 bispecifics; however, this may be in part explained by the elderly nature and high rate of refractoriness to chemotherapy noted in this patient population.

Acute immune reactions leading to transient cytokine release are a frequent side effect of T-cell-engaging therapies. Premedication with steroids and step-up dosing have been used to mitigate the occurrence of CRS following treatment with CD20xCD3 bispecific antibodies, whether the route of administration is intravenous or subcutaneous. In the present study, step-up dosing and additional prophylactic measures were systematically introduced over the course of the study and effectively mitigated the risk of CRS. In most cases, CRS was confined to cycle 1, low in grade (1 or 2), and resolved with supportive care without sequelae. No patients discontinued treatment, or experienced significant treatment delays, due to CRS. The frequency of grade CRS following intravenous treatment with odronextamab was 7%. All grade CRS events occurred prior to the optimization of the step-up dosing schedule to include three steps during cycle 1. The reported rate of grade CRS is similar to the incidence reported with other intravenously administered CD20xCD3 bispecific antibodies. In a phase 1 study of intravenously administered glofitamab in patients with R/R B-NHL, grade CRS was observed in 6% of patients treated at the RP2D.2 Reported rates for grade CRS with intravenous mosunetuzumab, plamotamab and IGM-2323 range between 1% and 6%. Epcoritamab has also been investigated in R/R B-NHL and no grade CRS events have been reported to date in a phase ½ study employing a subcutaneous route of administration.

CAR T therapy is a foundational treatment in the management of R/R B-NHLs, and emerging data suggest that this could become a new approach for some DLBCL patients at first relapse. However, many patients remain unsuitable candidates for CAR T therapy upon relapse due to having a rapidly progressive phenotype, and the requirements for apheresis, ex-vivo genetic manipulation and expansion of T-cells, and intensive preconditioning prior to dosing which may lead to prolonged cytopenias. Since both CD20xCD3 bispecifics and CAR T therapies re-direct T cells to trigger malignant B-cell killing, early toxicities such as CRS are observed with both classes of therapy. In this study, patients receiving odronextamab were hospitalized for observation during cycle 1 step-up dosing when CRS events most commonly occur. As data mature from ongoing studies implementing a modified step-up regimen (as detailed in Examples 1 and 2), the requirements for hospitalization may be reassessed, and administration of odronextamab entirely within the outpatient setting may be feasible.

In patients with B-NHL, neurotoxicity has been observed with T-cell-engaging therapies. The phase 1 study of glofitamab reported ICANS-like events in 5% of patients using a sponsor-derived list of 12 terms, and the reported incidence of neurologic symptoms in the phase ½ study of epcoritamab was 6%. Following treatment with odronextamab, CNS/ICANS-like events were primarily grade 1 or 2 and only four patients (3%) experienced a grade 3 event. No patients experienced grade 4 or 5 neurologic TEAEs, or discontinued odronextamab treatment due to neurologic TEAEs. Since there are no standardized criteria for reporting ICANS-like events, the utility of cross-study comparisons is somewhat limited.

There remains a very high unmet need for safe, effective, and accessible treatment options for patients with R/R B-NHL. This unmet need is amplified in the setting of patients who progress after CAR T therapy, where prognosis remains dismal with salvage therapy. This study provides evidence that odronextamab can confer durable, clinically relevant benefit in heavily pre-treated patients with R/R B-NHL, including in patients who have progressed after CAR T therapy.

In this study, treatment with odronextamab at therapeutically relevant doses ≥80 mg was associated with an ORR of 53% (all CRs) in patients with R/R DLBCL and no prior CAR T therapy, with 88% of CRs estimated to be ongoing at 12 months. Importantly, although ORR and CR rates (33% and 27%, respectively) were lower in the DLBCL patients who had progressed following prior CAR T therapy, 100% of complete remissions were estimated to remain ongoing at 12 months. These results may signal a clinically important advance in a post-CAR T setting where few options exist beyond salvage chemotherapy.

Early observations from dose-escalation studies with CD20xCD3 bispecifics have been reported in patients with R/R DLBCL. Recently, IV glofitamab has demonstrated an ORR and CR rate of 79% and 71%, respectively, in 14 patients treated at the recommended dose. Results have also been reported with subcutaneous epcoritamab, demonstrating an ORR and CR rate of 91% and 55%, respectively, in 11 patients treated at doses of 48 mg and higher. In the first-in-human study of plamotamab, the ORR among all patients with non-Hodgkin lymphoma was 43%, while that for the subgroup of patients with DLBCL was 38%. Preliminary data for IGM-2323 demonstrate an ORR of 31% in patients with DLBCL. Longer-term follow-up is required to assess the durability of responses and CRs for these agents.

CAR T therapy has recently become available as an option for FL patients who have progressed after at least two prior lines of therapy. The recent approval of axicabtagene ciloleucel in this patient population was supported by the results of the single-arm ZUMA-5 study. In this study, axicabtagene ciloleucel therapy demonstrated an ORR of 91%, and 60% were complete responses. The rates of ongoing remissions at 12 and 18 months were 76% and 74%, respectively.

Although CAR T therapies represent an important advance in the management of B-NHLs, not all FL patients may be eligible to receive this treatment due to the potential for severe toxicities, complexities in manufacturing and potential barriers to access in the community. In the present study, odronextamab demonstrated an ORR of 91%, and a CR rate of 72% at doses ≥5 mg in patients with R/R FL, the majority of whom were heavily pre-treated. Importantly, the rate of ongoing complete remission at 48 months was estimated to be 54%. This is the first study of a CD20xCD3 bispecific antibody to demonstrate persistence of remissions in over half of complete responders at 4 years. Odronextamab administration may offer an important off-the-shelf and convenient option for R/R FL patients who might be considered candidates for CAR T therapy after progressing on two or more lines of therapy. Other studies have reported early experience with CD20xCD3 bispecifics in R/R FL patients. Intravenous mosunetuzumab has recently reported ORR and CR rates of 79% and 58%, respectively, and early data from a first-in-human study of subcutaneous epcoritamab have reported ORR and CR rates of 90% and 50%, respectively, in 10 evaluable R/R FL patients. Longer term follow-up from these studies is awaited.

Patients treated in this study received a median of 10 doses of odronextamab therapy, inclusive of step-up doses. Although the treatment regimen was revised during dose expansion to allow treatment until progression, some patients were able to achieve durable responses with a shorter duration of treatment. The optimal duration of therapy for this class of agent is yet to be determined. It is unknown whether clinical outcomes differ between patients receiving limited cycles of therapy followed by an option for re-treatment or patients receiving continuation of therapy until progression. In this study, a subset of patients received limited cycles of treatment and seven were re-treated upon progression, mostly at lower doses. No patients experienced a response upon retreatment. A recent study suggests that loss of CD20 may be associated with acquired resistance to odronextamab. Although most of the patients received retreatment at low doses, acquired resistance including loss of CD20 may in some cases explain a lack of observed efficacy on retreatment, thus supporting the recommended treatment paradigm of continued dosing. Further studies are required to delineate mechanisms of resistance for the class of CD20xCD3 bispecifics that can inform future clinical studies.

The development of the CD20xCD3 bispecific antibody drug class heralds enormous potential to improve the lives of patients with B-NHLs, and is an intense area of focus in ongoing clinical trials. Odronextamab monotherapy continues to be investigated, as detailed in Examples 1 and 2. Although the results reported to date show very encouraging monotherapy activity with a manageable tolerability profile, chemotherapy-free regimens combining odronextamab with other immune modulators such as checkpoint inhibitors or co-stimulatory antibodies may present an important opportunity for improving outcomes for the hardest-to-treat patients with B-NHL. In summary, odronextamab has demonstrated promising clinical activity, manageable safety and durable responses in heavily pre-treated, highly refractory patients with B-NHLs. Odronextamab has the potential to provide off-the-shelf convenience for B-NHL patients, including those with rapidly progressive disease, and further investigation of odronextamab as monotherapy and in combination with other modalities is supported.

### Example 5: Clinical Evaluation of Anti-CD3 x Anti-CD20 Bispecific Antibody in Patients with CD20+ B-Cell Malignancies Previously Treated with CD20-Directed Antibody Therapy

This is an open-label, multi-center study of odronextamab administered as a subcutaneous (SC) injection in patients with relapsed/refractory B-cell malignancies. The SC study of odronextamab for B-NHL will include:
Dose finding cohorts for patients with follicular lymphoma (FL) grade 1-3a and diffuse large B-cell lymphoma (DLBCL)
Expansion cohorts for exploring dosing frequency for FL grade 1-3a and DLBCL

The total duration of study participation for each patient will vary based on the occurrence of 1 or more of the following: disease progression, withdrawal of consent, other study withdrawal criterion is met, or death.

The study will consist of 3 periods, including: Screening Period (up to 28 days): The screening period begins with the signing of the informed consent form (ICF) and ends when the patient has been confirmed to be eligible for the study and initiates treatment, or with the determination that the patient is ineligible and has been designated as a screen failure.

Treatment Period: The treatment period begins with the initiation of treatment and will last until the time of disease progression or other protocol-defined reason for treatment discontinuation. The frequency of dosing varies by cohort.

Follow-Up Period: Following treatment discontinuation, patients will be followed for safety and efficacy in the initial 90-days post last dose (safety follow-up period) or until they start another anti-lymphoma therapy, whichever is earlier. Patients who discontinue treatment for reasons other than disease progression will continue to be followed with extended follow-up after the safety follow-up (disease response assessments Q12W until end of year 1 followed by assessments every 6 months after that). After extended follow-up, all study patients will be followed at Q12W intervals for survival status until the time of death, loss to follow-up, patient withdrawal of consent for follow-up, or study termination by the sponsor, whichever is earlier.

Up to 102 patients are planned. The actual sample size will depend on the number of observed patients with DLTs and if any additional step-up dosing regimens are implemented. Patients of the same disease sub-type/regimen enrolled in dose finding portion at the recommended phase II dose (RP2D) will be counted in the corresponding expansion cohort for analysis.
In the dose finding portion, approximately 24 patients will be enrolled assuming a maximum of 2 dose-finding regimens are evaluated for FL and DLBCL each. Each dose finding regimen disease cohort will enroll up to 6 patients.
In the expansion portion, each cohort will enroll a total of 15 patients (including those added from dose finding cohort)
   In FL expansion, up to 24 patients with FL grade 1-3a will be enrolled in 2 cohorts (6 patients from FL dose finding cohort will be counted in the FL cohort 1 for analysis)
   In DLBCL expansion, up to 54 patients with DLBCL will be enrolled in 4 cohorts (6 patients from DLBCL dose finding cohort will be counted in the DLBCL cohort 1 for analysis)

Patients must have documented CD20+ B-cell malignancy, with active disease that has relapsed after or refractory to prior therapy, for whom no standard of care options exists, and for whom treatment with an anti-CD20 antibody may be appropriate.

Patients will receive odronextamab at the assigned dose in 21-day cycles. Cycle 1 will include weekly step-up doses until the step-up regimen is completed. The step-up regimen will include an initial dose and intermediate doses 1 and 2. Step-up dosing in cycle 1 will be followed by treatment cycles at full dose, every 21 days, until the time of disease progression or other protocol-defined reason for treatment discontinuation. During the treatment period, the dosing regimen (frequency of dosing) is variable by cohort. A cycle length is defined as 3 weeks (21 days), unless indicated otherwise. For Cycle 1, the cycle length could be extended until the step-up regimen is completed. The step-up regimen consists of an initial dose and intermediate doses 1 and 2.

For FL: The treatment period will comprise a 3-week step-up regimen with cycle 1 dosing on day 1, day 8, and day 15 or until the step-up regimen is completed followed by Q3W dosing from cycle 2-cycle 8 (21-day cycles with dosing on day 1 of each cycle), followed by extended treatment from cycle 9 onwards. Extended treatment cycles will continue until disease progression or other protocol-defined reason for treatment discontinuation, and will have the following dosing frequency from cycle 9 onwards in the FL dose finding and expansion cohorts (FL cohort 1 and FL cohort 2):
FL Dose finding cohort and FL cohort 1: 21-day cycles with 400 mg SC on day 1 of each cycle.
FL cohort 2: 8-week cycles with 400 mg SC on day 1 of each cycle

For DLBCL (with and without prior CAR-T): The treatment period will comprise of a 3-week step-up regimen during cycle 1 with dosing on day 1, day 8, and day 15 or until the step-up regimen is completed, followed by full dose on cycle 2 day 1, day 8, and day 15, followed by extended treatment cycles per the following cohorts (DLBCL dose finding cohort and expansion cohorts [DLBCL cohort 1-4]), from cycle 3 onwards, until disease progression or other protocol-defined reason for treatment discontinuation:
DLBCL dose finding cohort and DLBCL cohort 1: 21-day cycles with 400 mg SC on day 1 of each cycle DLBCL cohort 2: 21-day cycles with 600 mg SC on cycle day 1.
DLBCL cohort 3: 21-day cycles with 400 mg SC on cycle 3 day 1, day 8, and day 15, followed by 400 mg SC on day 1 of each cycle from cycle 4 onwards
DLBCL cohort 4 (DLBCL after failure of CAR-T): 21-day cycles at the RP2D (selected from cohorts 1-3). This cohort will be opened if there are less than 15 patients with DLBCL post CAR-T in expansion cohorts 1-3 combined.

Inclusion Criteria

A patient must meet the following criteria to be eligible for inclusion in the study:
1. Have documented CD20+ B-cell malignancy, with active disease not responsive to prior therapy, for whom no standard of care options exists, and for whom treatment with an anti-CD20 antibody may be appropriate: Confirmed diagnosis of B-NHL requiring therapy as defined by WHO classification 2017
   NOTE: A patient with CD20-negative lymph node (NHL) biopsy performed as standard of care just prior to enrollment, remains eligible for the study provided the patient had previously documented CD20+ disease AND was previously treated with rituximab or other CD20-directed antibody therapy within approximately 6 months.
2. Patients with B-NHL must have had prior treatment with an anti-CD20 antibody therapy. Refractory is defined as no response (SD/PD) or relapse within months of last treatment.
   For inclusion in the FL grade 1-3a expansion cohort, patients must have received at least 2 prior lines of systemic therapy, including an anti-CD20 antibody and an alkylating agent; patients require failure of combination of lenalidomide plus rituximab where approved or not appropriate to receive this treatment according to the investigator.
   For the inclusion in the disease-specific expansion cohort enrolling DLBCL patients after failure of CAR-T therapy, the patient must have recovered from the toxicities of the lymphodepletion therapy and CAR-T infusion. There is no requirement for the prior CAR-T therapy to be the most recent line of therapy before study enrollment. Prior to enrollment, there must be verbal communication and documentation between the investigator and the medical monitor regarding prior toxicities associated with CAR-T therapy and their resolution.
   Patients with aggressive lymphoma assigned to the aggressive lymphoma expansion cohort must have received at least one prior line of therapy consisting of an anti-CD20 antibody. Patients with prior CAR-T therapy will not be included in this cohort.
   Patients with FL grade 1-3a and DLBCL must have received at least 2 prior lines of systemic therapy, including an anti-CD20 antibody and an alkylating agent.
3. All patients must have at least one bi-dimensionally measurable lesion 1.5 cm on the longest diameter) documented by CT or MRI scan, if CT scan is not feasible.
4. Age ≥18 years
5. Eastern Cooperative Oncology Group (ECOG) performance status ≤1
6. Life expectancy of at least 6 months
7. Adequate bone marrow function documented by:
   a. Platelet counts ≥75×10⁹/L
   b. Hb level ≥9 g/dL
   c. ANC≥1×10⁹/L
   NOTE: Patients with cell counts below thresholds listed above may be considered for enrollment.
      Patients with bone marrow involvement or splenic sequestration should meet the following hematologic parameters:
         Platelet count ≥25×10⁹/L. A patient may not have received platelet transfusion therapy within 3 days prior to first dose of odronextamab in order to meet the platelet eligibility criterion.
         Hemoglobin ≥7.0 g/dL
         Absolute neutrophil count (ANC)≥0.5×10⁹/L. A patient may not have received granulocyte colony stimulating factor within 2 days prior to first dose of odronextamab in order to meet the ANC eligibility criterion.
8. Adequate organ function documented by:
   Aspartate aminotransferase (AST) and alanine aminotransferase (ALT)≤2.5×ULN
   Total bilirubin ≤1.5×ULN
   NOTE: Patients with Gilbert's syndrome do not need to meet this requirement provided their total bilirubin is unchanged from their baseline.
   Calculated creatinine clearance by Cockcroft-Gault 50 mL/min
   NOTE: Patients may be considered for enrollment if, in the opinion of the investigator, the abnormal laboratory results are due to underlying disease.
   NOTE: Patients with borderline creatinine clearance by Cockcroft-Gault may be considered for enrollment if a measured creatinine clearance (based on 24-hour urine or other reliable method) is ≥50 mL/min.
9. Willingness to undergo mandatory tumor biopsy pre-treatment, if in the opinion of the investigator, the patient has an accessible lesion that can be biopsied without significant risk to the patient.

Exclusion Criteria

A patient who meets any of the following criteria will be excluded from the study:
1. Primary central nervous system (CNS) lymphoma or known or suspected CNS involvement by non-primary CNS NHL
2. History of or current relevant CNS pathology such as:
   Epilepsy, seizure, paresis, aphasia, apoplexia, severe brain injuries, cerebellar disease, organic brain syndrome, psychosis, or
   Evidence for presence of inflammatory lesions and/or vasculitis on cerebral MRI
3. Standard anti-neoplastic chemotherapy (non-biologic) within 5-times the half-life or within 28 days, whichever is shorter, prior to first administration of study drug.
4. Standard radiotherapy within 14 days of first administration of study drug.
   NOTE: Palliative radiotherapy to a symptomatic lymph node/lesion is allowed provided the irradiated lesion(s) or node(s) is not included as a target lesion for tumor assessments
5. Allogeneic stem cell transplantation
6. Treatment with rituximab, alemtuzumab, or other investigational or commercial biologic agent within 12 weeks prior to first administration of study drug.
7. Immunosuppressive therapy (other than biologic) within 28 days of first administration of study drug.
8. Treatment with an investigational non-biologic agent within 28 days of first administration of study drug.
9. History of allergic reactions attributed to compounds of similar chemical or biologic composition of study drug.
10. History of hypersensitivity to any compound in the tetracycline antibiotics group.
11. Concurrent active malignancy for which the patient is receiving treatment.
12. Known active bacterial, viral, fungal, mycobacterial or other infection or any major episode of infection requiring hospitalization or treatment with IV anti-infectives within 4 weeks of first administration.
13. Evidence of significant concurrent disease or medical condition that could interfere with the conduct of the study, or put the patient at significant risk including, but not limited to, significant cardiovascular disease (eg, New York Heart Association Class III or IV cardiac disease, myocardial infarction within the previous 6 months, unstable arrhythmias or unstable angina) and/or significant pulmonary disease (eg, obstructive pulmonary disease and history of symptomatic bronchospasm).
   NOTE: Patients with a medical history of cardiac disease should be evaluated by ECHO or multi-gated acquisition scan (MUGA) prior to first administration of odronextamab to ensure adequate cardiac reserves and function.
14. Ongoing systemic corticosteroid treatment, with the exception of corticosteroid use for other (non-tumor and non-immunosuppressive) indications up to a maximum of 10 mg/day of prednisone or equivalent.
15. Infection with human immunodeficiency virus (HIV) or chronic infection with hepatitis B virus (HBV), hepatitis C virus (HCV), or cytomegalovirus (CMV) infection (as noted by detectable levels on blood PCR assay).
   a. Patients with hepatitis B (HepBsAg+) who have controlled infection (serum hepatitis B virus DNA that is below the limit of detection AND receiving anti-viral therapy for hepatitis B) are permitted upon consultation with the physician managing the infection.
   b. Patients who show detectable levels of CMV at screening will need to be treated with appropriate antiviral therapy and demonstrate at least 2 undetectable levels of CMV by PCR assay (at least 7 days apart) before being re-considered for eligibility.
16. Known hypersensitivity to both allopurinol and rasburicase.
17. Pregnant or breast-feeding women.
18. Women of childbearing potential* or men who are unwilling to practice highly effective contraception prior to the initial study drug treatment, during the study, and for at least 6 months after the last dose.
19. Administration of live vaccination within 28 days of first administration of study drug.

Two extended treatment dosing regimens for FL and three dosing regimens for DLBCL are being investigated in this study to evaluate the frequency of SC dosing. The proposed SC dosing regimens include a step up-dosing (2 mg/26 mg/100 mg) in cycle 1, 400 mg in cycle 2 (full dose), and 400 mg or 600 mg in later cycles (Table 7 and Table 8).

TABLE 7
Odronextamab SC Regimens in FL Grade 1-3a Expansion Cohorts

| | | | | | | | | | | Cycle* 9 |
|---|---|---|---|---|---|---|---|---|---|---|
| | | | | | | | | | | and above |
| | Cycle* 1 | | | Cycle* 2 | | | Cycle* 3-8 | | | (W25+) |
| | Dosing Day | | | | | | | | | |
| FL Cohort | D 1 | D 8 | D 15 | D 1 | D 8 | D 15 | D 1 | D 8 | D 15 | D 1 |
| FL Cohort 1 | 2 mg | 26 mg | 100 mg | 400 mg | — | — | 400 mg | — | — | 400 mg |
| | | | | | | | | | | Q3W |
| FL Cohort 2 | 2 mg | 26 mg | 100 mg | 400 mg | — | — | 400 mg | — | — | 400 mg |
| | | | | | | | | | | Q8W |

*1 cycle = 21 days unless indicated

TABLE 8
Odronextamab SC Regimens in DLBCL Expansion Cohorts

| | | | | | | | | | | Cycle* 4 and | | |
|---|---|---|---|---|---|---|---|---|---|---|---|---|
| | Cycle* 1 | | | Cycle* 2 | | | Cycle* 3 | | | above (W10+) | | |
| DLBCL | Dosing Day | | | | | | | | | | | |
| Cohort | D 1 | D 8 | D 15 | D 1 | D 8 | D 15 | D 1 | D 8 | D 15 | D 1 | D 8 | D 15 |
| DLBCL | 2 mg | 26 mg | 100 mg | 400 mg | 400 mg | 400 mg | 400 mg | — | — | 400 mg | — | — |
| Cohort 1 | | | | | | | | | | | | |
| DLBCL | 2 mg | 26 mg | 100 mg | 400 mg | 400 mg | 400 mg | 600 mg | — | — | 600 mg | — | — |
| Cohort 2 | | | | | | | | | | | | |
| DLBCL | 2 mg | 26 mg | 100 mg | 400 mg | 400 mg | 400 mg | 400 mg | 400 mg | 400 mg | 400 mg | — | — |
| Cohort 3 | | | | | | | | | | | | |
| DLBCL | 2 mg | 26 mg | 100 mg | 400 mg | 400 mg | 400 mg | RP2D | | | | | |
| Cohort 4 | | | | | | | | | | | | |
| (after failure | | | | | | | | | | | | |
| of CAR-T) | | | | | | | | | | | | |

*1 cycle = 21 days

If in either SC-1N or SC-2N cohort (combined), two or more patients experience with grade≥3 CRS, depending on the dose following which the events of CRS occur, the step-up regimen will be modified as follows (Table 9 and Table 10).

TABLE 9
Dose Finding for FL Grade 1-3a (SC Administration)
Treatment period

| | Cycle 1 | | | | | Cycle 3+ |
|---|---|---|---|---|---|---|
| | Step-up Regimen* | | | Cycle 2 | | Q3W |
| Cohort | D 1 | D 8 | D 15 | D 1 | D 8 | D 1 |
| | 2 mg | 26 mg | 100 mg | 400 mg | n/a | 400 mg |

Alternate step-up regimens (below) will be selected if two or more patients with grade ≥3 CRS†

| | | | | | | |
|---|---|---|---|---|---|---|
| SC-1Na | 2 mg | 26 mg | 50 mg | 400 mg | n/a | 400 mg |
| SC-1Nb | 2 mg | 26 mg | 50 mg | 200 mg | 400 mg | 400 mg |
| SC-1Nc | 2 mg | 10 mg | 50 mg | 200 mg | 400 mg | 400 mg |
| SC-1Nd | 1 mg | 10 mg | 50 mg | 200 mg | 400 mg | 400 mg |

*The initial (cycle 1) step-up regimen may be extended with weekly step-up doses until the step-up regimen is completed.
†If SC-1N or SC-2N (combined) results in two or more patients with grade ≥3 CRS, then the step-up regimen will be modified for dose finding cohorts in both subtypes.

TABLE 10
Dose Finding for DLBCL (SC Administration)
Treatment period

| | Cycle 1 | | | | | | Cycle 3+ |
|---|---|---|---|---|---|---|---|
| | Step-up Regimen* | | | Cycle 2 | | | Q3W |
| Cohort | D 1 | D 8 | D 15 | D 1 | D 8 | D 15 | D 1 |
| SC-2N | 2 mg | 26 mg | 100 mg | 400 mg | 400 mg | 400 mg | 400 mg |

Alternate step-up regimens (below) will be selected if two or more patients with grade ≥3 CRS†

| | | | | | | | |
|---|---|---|---|---|---|---|---|
| SC-2Na | 2 mg | 26 mg | 50 mg | 400 mg | 400 mg | 400 mg | 400 mg |
| SC-2Nb | 2 mg | 26 mg | 50 mg | 200 mg | 400 mg | 400 mg | 400 mg |
| SC-2Nc | 2 mg | 10 mg | 50 mg | 200 mg | 400 mg | 400 mg | 400 mg |
| SC-2Nd | 1 mg | 10 mg | 50 mg | 200 mg | 400 mg | 400 mg | 400 mg |

*The initial (cycle 1) step-up regimen may be extended with weekly step-up doses until the step-up regimen is completed.
†If SC-2N or SC-1N (combined) results in two or more patients with grade ≥3 CRS, then the step-up regimen will be modified for both sub-type dose finding cohorts.

In preclinical studies in cynomolgus monkeys, SC administration of odronextamab was associated with lower serum cytokine levels and induced sustained B-cell depletion to a similar level as IV administration. Thus, it is anticipated that SC administration of odronextamab in patients may simplify the drug administration process, improve overall patient convenience, and enhance tolerability by further attenuating cytokine release.

TABLE 11
CRS Toxicity Grading

| CRS | | | | |
|---|---|---|---|---|
| Parameter | Grade 1 | Grade 2 | Grade 3 | Grade 4 |
| Fever¹ | Temperature ≥ 38° C. | Temperature ≥ 38° C. | Temperature ≥ 38° C. | Temperature ≥ 38° C. |

With

| | | | | |
|---|---|---|---|---|
| Hypotension | None | Not requiring | Requiring a vasopressor | Requiring multiple |
| | | vasopressors | with or without | vasopressors |
| | | | vasopressin | (excluding vasopressin) |

And/or²

| | | | | |
|---|---|---|---|---|
| Hypoxia | None | Requiring low-flow | Requiring high-flow nasal | Requiring positive |
| | | nasal cannula³ or | cannula³, facemask, | pressure (eg CPAP, |
| | | blow-by | nonrebreather mask, or | BiPAP, intubation and |
| | | | Venturi mask | mechanical ventilation) |

CPAP = continuous positive airway pressure, bilevel positive airway pressure, BiPAP = bilevel positive airway pressure.
Organ toxicities associated with CRS may be graded according to CTCAE v4.03 but they do not influence CRS grading.
¹Fever is defined as temperature ≥ 38° C. not attributable to any other cause. In patients who have CRS then receive antipyretic or anti-cytokine therapy such as tocilizumab or steroids, fever is no longer required to grade subsequent CRS severity. In this case, CRS grading is driven by hypotension and/or hypoxia.
²CRS grade is determined by the more severe event: hypotension or hypoxia not attributable to any other cause. For example, a patient with temperature of 39.5° C., hypotension requiring 1 vasopressor, and hypoxia requiring low-flow nasal cannula is classified as grade 3 CRS.
³Low-flow nasal cannula is defined as oxygen delivered at ≤6 L/minute. Low flow also includes blow-by oxygen delivery, sometimes used in pediatrics. High-flow nasal cannula is defined as oxygen delivered at >6 L/minute.

### Example 6: Premedications Applied to Odronextamab SC Administration

The following premedications apply to odronextamab from the initial dose through the first full dose. If the patient has CRS of any grade with the first full dose, premedications are continued until the full dose is tolerated without experiencing CRS:
1. 12 to 24 hours prior to planned start time of SC injection during step-up dosing and first full dose of odronextamab:
   a. Dexamethasone 10 mg oral (PO) or equivalent dose of steroid
2. On the day of SC injection during step-up dosing and first full dose of odronextamab:
   a. Dexamethasone 20 mg IV or PO 1 to 3 hours prior to SC injection on the day of treatment
   b. Diphenhydramine 25 mg IV or PO 30 to 60 minutes before (can be replaced with another equivalent antihistamine)
   c. Acetaminophen 650 mg PO 30 to 60 minutes before, unless the patient has received it within the past 4 hours prior to injection with odronextamab, or is allergic to acetaminophen
3. 24 (±4) hours after odronextamab injection during step-up dosing and first full dose of odronextamab:
   a. Dexamethasone 10 mg PO or equivalent dose of steroid

First dose administration following full dose administered without experiencing CRS of any grade with 20 mg of dexamethasone IV or PO
4. Premedication for day of SC injection of odronextamab following first full dose:
   a. Dexamethasone 10 mg IV or PO 1 to 3 hours prior to SC injection on the day of treatment
   b. Diphenhydramine 25 mg IV or PO 30 to 60 minutes before (can be replaced with another equivalent antihistamine)
   c. Acetaminophen 650 mg PO 30 to 60 minutes before, unless the patient has received it within the past 4 hours prior to SC injection with odronextamab, or is allergic to acetaminophen

For subsequent doses, no premedication is required if the SC injection is tolerated without experiencing CRS of any grade with the reduced 10 mg of dexamethasone IV or PO.

### Example 7: Modeling and Simulation in Support of Odronextamab Subcutaneous Dose Selection for Adult Patients with Indolent or Aggressive Non-Hodgkin Lymphoma

Odronextamab is a hinge-stabilized, human CD20xCD3 IgG4-based bispecific antibody that binds CD20-expressing cells and CD3 on T cells, eliciting T-cell-mediated cytotoxicity independent of T-cell receptor-mediated recognition. Studies of intravenous (IV) odronextamab in patients with previously treated CD20-positive B-cell malignancies are ongoing and showed promising efficacy. The IV regimen includes step-up dosing in Cycle 1 with doses of 0.7/4/20 mg administered over 3 weeks (each dose split over 2 days), to mitigate the risk of cytokine release syndrome (CRS). Odronextamab is subsequently administered during Cycles 2-4 at 80 mg once weekly (QW) for indolent non-Hodgkin lymphoma (NHL) and at 160 mg QW for aggressive NHL. For further improvement of overall tolerability, patient convenience, and reduced hospital resource utilization, subcutaneous (SC) administration of odronextamab is being explored. This analysis, based on modeling and simulation, was performed to determine SC regimens for clinical investigation.

The analysis included 6 steps: 1) A population pharmacokinetic (PK) model was developed to estimate IV odronextamab PK parameters with observed concentration data from patients with B-cell NHL participating in the clinical trials described in Examples 1 and 2; 2) A projected absorption rate constant (based on data from Regeneron IgG4-based antibodies) and a projected SC bioavailability parameter (based on a study of SC odronextamab in cynomolgus monkeys) were added for the model to be used for SC PK profile simulation; 3) An exposure-response analysis was performed to identify target exposure parameters for clinical efficacy with IV regimens in patients with indolent and aggressive NHL; 4) SC PK profiles were simulated to identify regimens achieving target exposures for efficacy without exceeding maximum concentration (Cₘₐₓ) of the highest evaluated IV doses; 5) A quantitative systems pharmacology (QSP) model was developed to predict interleukin (IL)-6 profiles following SC step-up dosing; and 6) Odronextamab SC regimens were simulated for selection of those with IL-6 levels not predicted to exceed those of IV regimens.

Based on the modeling and simulation results, the proposed SC regimen selected for step-up dosing was 2/26/100 mg in Cycle 1 (no split dosing) for both indolent and aggressive NHL. The simulated median Cₘₐₓ during the first week was lower with 2 mg SC than with 0.7 mg IV with split dosing (0.2/0.5 mg). The predicted time to Cₘₐₓ was ˜4 days with SC, much later than with IV (where Cₘₐₓ occurs near the end of the infusion). A longer time to Cₘₐₓ may reduce the risk of CRS, which mostly occurs within 1-2 days after split-dose IV administration. The proposed SC dose is 400 mg every 3 weeks (Q3W) after Cycle 1 for indolent NHL, and 400 mg QW in Cycle 2 and 400 mg Q3W after Cycle 2 for aggressive NHL. Additional SC regimens for indolent and aggressive NHL were proposed for testing based on clinical observations.

According to the simulation results, the proposed SC regimens maintain the required efficacious odronextamab concentrations over the initial 4 cycles of treatment when most tumor responses are expected to occur. Simulated IL-6 profiles, obtained using the SC QSP model, showed the peak IL-6 values during Cycle 1 with the proposed SC step-up dosing regimen (2/26/100 mg) would not exceed those of 0.7/4/20 mg IV.

SC administration of odronextamab may be simpler and more convenient than IV dosing. PK and IL-6 modeling and simulation analyses enabled the identification of SC regimens for clinical evaluation, which may improve overall tolerability while preserving efficacy for the treatment of patients with B-cell NHL.

### Example 8: Selection of Odronextamab Pediatric Dosing Regimens for Aggressive Non-Hodgkin Lymphoma Via a Modeling and Simulation Approach

Odronextamab is a hinge-stabilized, human CD20xCD3 IgG4-based bispecific antibody that binds CD20-expressing cells and CD3 on T cells, targeting CD20+ cells via T-cell-mediated cytotoxicity, independent of T-cell receptor-mediated recognition. Clinical studies of odronextamab in adult patients with relapsed/refractory CD20+ B-cell malignancies are ongoing. Encouraging efficacy has been reported in patients. In the US, approximately 800 new cases of pediatric non-Hodgkin lymphoma (NHL) are diagnosed each year. Improved treatment options are needed for pediatric patients, particularly those with relapsed/refractory disease following chemoimmunotherapy, who have poor outcomes. A study of odronextamab in relapsed/refractory pediatric patients with B-cell NHL (B-NHL) is being planned. The objective of this analysis was to determine intravenous (IV) regimens for testing in pediatric patients that can achieve similar exposures to those achieved in adults.

The analysis was conducted in 6 steps: 1) A population pharmacokinetic (PK) model was developed with data from adults receiving IV odronextamab and assessed effects of body weight (WT) on drug clearance (CL) and volume of distribution (V); 2) A virtual pediatric population was created with demographics derived from the 2017-2018 National Health and Nutrition Examination Survey database; 3) Pediatric model parameters were extrapolated from those of adults by applying effects of WT and age on CL and V as appropriate, and a maturation effect on non-specific elimination was considered in addition to weight effects for infants younger than 1 year; 4) Odronextamab PK profiles were simulated in virtual pediatric patients aged 6 months to 18 years in 4 weight bands (≥40 kg, 20-39 kg, 10-19 kg, 6-9 kg) using the extrapolated pediatric population model parameters; 5) Odronextamab drug amounts were adjusted in each weight band for each weekly (QW) dosing period, and every 2 weeks (Q2W) dosing period with the aim of matching adult patient exposures; and 6) Pediatric regimens were selected for testing based on 2 criteria. Firstly, for safety, median maximal concentration (Cₘₐₓ) during Cycle 1 step-up dosing within 80-125% of the adult value. Secondly, for efficacy, median minimal concentration (Cₘᵢₙ) at steady state during Cycle 2 onwards not less than the adult value.

Odranextamab concentrations in serum following IV doses ranging from 0.03-320 mg in patients with relapsed/refractory B-NHL showed a bi-exponential decay, which was adequately described by the two-compartment model with parallel first-order elimination and modified Michaelis-Menten elimination terms. The latter is not only concentration dependent, but also time dependent. Body weight was found to be a significant covariant on linear CL and V. With PK parameter estimates, simulations were performed for odranextamab IV dosing regimens by weight bands for pediatric patients as shown in Table 12. As with the adult regimen, step-up dosing with split doses was implemented during cycle 1 to limit Cₘₐₓ values and mitigate the risk of CRS. Children with a body weight ≥40 kg would receive the adult regimen, while those weighing <40 kg would receive reduced doses.

The proposed regimens were predicted to achieve odronextamab exposures similar to those with the adult regimen during step-up doses (weeks 1-3) as well as weekly and biweekly treatment periods. No significant difference in Cₘₐₓ was predicted with the step-up doses; the ratios of median Cₘₐₓ for the pediatric WT groups relative to adults fell within 0.831-1.24. The median Cₘᵢₙ values at steady state were not below those observed in adults, and no significant differences in Cₘᵢₙ were observed across the weight groups.

Additional exploratory simulations were performed to assess uniform weight-based dosing (mg/kg) for the overall pediatric population. This approach was inferior to the regimens tiered by WT bands due to above-target concentrations among individuals weighing 20 kg and below-target concentrations in those weighing 6-9 kg.

The modeling and simulation approach allowed the identification of dosing regimens that might be suitable for use in children with B-NHL. Based on predicted PK profiles, pediatric dosing regimens tiered by WT bands should preserve the efficacy of odronextamab observed in adults, without increasing the risk of acute toxicity.

TABLE 12
Proposed Weight-Tier Dosing Regimen Based on Simulations in Virtual Patients

| | | | | | QW Dose | |
|---|---|---|---|---|---|---|
| | | Dose (Split) | Dose (Split) | Dose (Split) | (mg) | Q2W Dose |
| | Weight Range | (mg) | (mg) | (mg) | Weeks | (mg) > |
| Age | (kg) | Week 1 | Week 2 | Week 3 | 4-12 | Week 12 |
| ≥18 years | 40-164 | 0.7 (0.2/0.5) | 4 (2/2) | 20 (10/10) | 160 | 320 |
| <18 years | 40-164 | 0.7 (0.2/0.5) | 4 (2/2) | 20 (10/10) | 160 | 320 |
| <18 years | 20-39 | 0.4 (0.1/0.3) | 2 (1/1) | 12 (6/6) | 90 | 150 |
| <18 years | 10-19 | 0.3 (0.1/0.2) | 1.6 (0.8/0.8) | 8 (4/4) | 60 | 100 |
| <18 years | 6-9 | 0.24 (0.07/0.17) | 1.2 (0.6/0.6) | 6 (3/3) | 45 | 75 |

Doses administered on Weeks 1-3 were split over Day 1 and Day 2 of each week. Split doses (mg) are shown in parenthesis.
QW, once weekly; Q2W, once every 2 weeks; WT, body weight.

### Example 9: Intravenous Odronextamab Step-Up Regimen for Reducing the Risk of High-Grade Cytokine Release Syndrome

Odronextamab is a hinge-stabilized, human CD20xCD3 IgG4-based bispecific antibody that binds CD20-expressing cells and CD3 on T cells, targeting CD20+ cells via T-cell-mediated cytotoxicity, independent of T-cell receptor-mediated recognition. Transient cytokine release is common with T-cell engaging bispecific antibodies, particularly in the early weeks of treatment. This is associated with an increased risk of cytokine release syndrome (CRS), a serious and potentially life-threatening adverse event. As the intensity of cytokine release is positively associated with drug exposures, especially in the first 3 weeks of treatment, administration of lower doses prior to a full treatment dose (i.e. step-up dosing) is an approach to mitigate the risk of CRS.

Intravenous (IV) odronextamab has shown encouraging efficacy in patients with B-cell non-Hodgkin lymphoma (B-NHL). In general, CRS was low grade (Gr), with incidence of Gr in 7% of patients across B-NHL histologies despite implementation of a step-up dosing regimen (1 mg/20 mg in Weeks 1-2 with split doses over 2 days) and steroid prophylaxis. The aim of this work was to determine the step-up regimen to reduce the incidence of Gr≥3 CRS.

The following step-up dosing conditions were assessed: 1) The effect of dose on peak odronextamab concentrations in Weeks 1-3 including split dosing and addition of more intermediate doses via pharmacokinetics model-based simulations; 2) The projected time profiles of interleukin (IL)-6 (a surrogate cytokine associated with CRS) with a quantitative systems pharmacology (QSP) model under various dosing scenarios; 3) The effect of type and timing of premedication on the occurrence of CRS; and 4) The baseline cytokine levels as a risk factor for Gr CRS.

Based on the evaluations of multiple factors as described above, the step-up dosing regimen consists of 0.7 mg (0.2/0.5 mg split over 2 days) at Week 1; 4 mg (2/2 split) at Week 2, and 20 mg (10/10 split) at Week 3. Early administration of premedication (12-24 hours prior to first split dose and on the day of each split dose vs. only on the day of each split dose) significantly reduced baseline (pre-treatment) cytokine levels.

Compared with the previously tested regimen, the new regimen decreased the total initial dose (Week 1) by 30% (from 1 mg to 0.7 mg), with a more marked reduction of 60% on Day 1 (from 0.5 mg to 0.2 mg). This resulted in a mean observed peak drug concentration reduction of ˜50% on Day 1 and a median peak IL-6 concentration post-treatment of 21.1 pg/mL (range 0.6-1163), corresponding to a 17% reduction compared with that from 0.5 mg on Day 1 (median 25.5 pg/mL [range 0.8-3560]).

The dose reduction on Week 1 Day 1 and the addition of the 4 mg intermediate dose at Week 2 reduced the intensity of cytokine release compared with the previously tested 1 mg (0.5/0.5)/20 mg (10/10) doses, according to the QSP simulation. This new regimen has been tested in clinical studies in patients with follicular lymphoma and diffuse large B-cell lymphoma. As of April 2022, there have been no Gr≥3 CRS in the first 100 patients treated with this regimen.

Importantly, although the odronextamab IV step-up dosing regimen showed a lower concentration of odronextamab compared with the original regimen during Weeks 1-3, the concentrations were similar after the first full dose was administered, with similar trough concentrations after Week 4. This indicates that the same therapeutic levels are achieved with both regimens, which is beneficial for the treatment of disease.

This odronextamab dosing regimen was associated with lower risk of CRS and lower levels of baseline cytokine levels compared with the original regimen. The drug exposure was lower in the first 3 weeks when the risk of CRS is greater, but it was comparable to that of the original regimen after the full dose of treatment was received, ensuring that odronextamab dose levels required for efficacy are maintained.

### Example 10: Quantitative Systems Pharmacology Modeling Framework for Evaluation of Cytokine Release Mediated by Intravenous Odronextamab Monotherapy in Patients with B-Cell Non-Hodgkin Lymphoma

Odronextamab is a hinge-stabilized, human CD20xCD3 IgG4-based bispecific antibody that binds CD20-expressing cells and CD3 on T cells, targeting CD20+ cells via T-cell-mediated cytotoxicity, independent of T-cell receptor-mediated recognition. T-cell activation following odronextamab administration can cause a temporary increase in the levels of circulating cytokines, and this has been associated with a risk of cytokine release syndrome (CRS). CRS is a recognized safety concern with all T-cell engaging bispecific antibodies, which mostly occurs during the first cycle of treatment.

This study was performed to evaluate cytokine profiles, especially interleukin (IL)-6 (a surrogate cytokine associated with CRS) in patients with B-cell non-Hodgkin lymphoma (B-NHL) receiving intravenous (IV) odronextamab monotherapy. Quantitative systems pharmacology (QSP) modeling is an emerging mathematical modeling approach that is based on and integrates our understanding of disease biology and the mechanism of action of the drug of interest. A range of questions arising from drug research and clinical development may be evaluated and addressed using the QSP approach. The objective of this work was to determine the step-up dosing regimen for achieving improved safety profiles using a QSP model.

Odronextamab pharmacokinetics (PK) data, cellular dynamics of T cells and B cells, and disease characteristics of patients with B-NHL were used to develop the QSP model. In order to describe the IL-6 profiles, a semi-mechanistic model was adapted and modified. A time-variant negative feedback loop was incorporated to account for the observed attenuation of IL-6 release following repeated doses. The model parameters were created with in vivo and in vitro preclinical data and relevant literature information and calibrated with clinically observed IL-6 data from patients treated with odronextamab monotherapy, observed odronextamab concentrations in serum, B-cell counts, CD8+ T-cell counts, and tumor size data obtained from a clinical study.

The calibrated model was used to assess time profiles of IL-6 release associated with different odronextamab dosing regimens. These assessments were performed using a virtual population of patients with B-NHL (n=300 per dosing regimen).

The calibrated QSP model was used to simulate IL-6 profiles with different proposed step-up dosing scenarios, including testing different dose levels in Weeks 1-4, with or without splitting the dose in each week, the ratio of splitting the dose over 2 days in each week, and the step-up dosing period from 2-4 weeks prior to giving a full dose of the treatment. Simulations suggested that splitting the dose at Week 1, Week 2, and Week 3 reduces the likelihood of IL-6 release in the early weeks of treatment. The highest IL-6 peak is expected to occur in Week 1 and an uneven split ratio (0.2/0.5 mg) of a 0.7 mg dose over 2 days of Week 1 would mediate the highest IL-6 release on day 1 while maintaining a similar or lower IL-6 release on day 2. As IL-6 release in Week 2 could be high, a low dose of 4 (2/2 split) mg is predicted to release less IL-6 than doses >4 mg. After comparison of simulation results for various dosing scenarios, a step-up dosing regimen with 0.7 (0.2/0.5) mg at Week 1, 4 (2/2) mg at Week 2, and 20 (10/10) mg at Week 3 was identified for testing in clinical trials.

In addition, the QSP model included variability in PK parameters of odronextamab and could predict PK profiles of odronextamab in the virtual B-NHL population, which showed comparable exposures with observed odronextamab concentrations following the previously tested step-up dosing regimen of 1 mg (Week 1), 20 mg (Week 2), and 160 mg (Week 3). The model was also able to predict the CD8+ T-cell and B-cell profiles over time following IV odronextamab split-dosing regimen.

This QSP model was developed to address the safety concern related to CRS following treatment with odronextamab. The work demonstrated that the QSP modeling is a powerful tool that enabled determination of odronextamab step-up dosing to minimize the risk of higher grade (i.e. grade 2 and 3) CRS in lymphoma patients treated with odronextamab.

### Example 11: Evaluation of Dynamics of IL-6 Release During Step-Up Dosing of Subcutaneous Administration of Odronextamab Via a Quantitative Systems Pharmacology Modeling Approach

Odronextamab is a hinge-stabilized, human CD20xCD3 IgG4-based bispecific antibody that binds CD20-expressing cells and CD3 on T cells, eliciting T-cell-mediated cytotoxicity independent of T-cell receptor-mediated recognition. T-cell activation following odronextamab administration can result in a transient but clinically significant increase in circulating cytokine concentrations. This may lead to cytokine release syndrome (CRS), 1 of the most important safety concerns with T-cell engaging therapies. As most cytokine release occurs during the early weeks of treatment, a step-up dosing strategy prior to administration of a full treatment dose appears to be a successful strategy to mitigate the level of cytokine release and attenuate the occurrence of CRS.

This study was performed to evaluate interleukin (IL)-6 profiles following odronextamab subcutaneous (SC) injection during cycle 1 step-up dosing (Weeks 1-3) in patients with B-cell non-Hodgkin lymphoma (B-NHL). A quantitative systems pharmacology (QSP) model, which is an emerging mathematical modeling approach that can help improve our understanding of drug-disease interactions and can be used to evaluate hypothetical scenarios in preclinical and clinical settings, was developed and applied to identify SC step-up doses of odronextamab suitable for investigating in clinical studies.

A QSP model that can describe IL-6 profiles (a surrogate cytokine associated with CRS) following intravenous (IV) odronextamab was previously developed and calibrated using pharmacodynamic data from a clinical study (Example 10). The IV QSP model was updated by adding relevant components (e.g. drug absorption compartment, lymph node compartment) to enable the description of IL-6 profiles following SC administration of odronextamab. Model parameters were calibrated using IL-6 concentration data of other CD20xCD3 bispecific antibodies under SC administration reported in the literature.

Odronextamab SC step-up doses of interest were proposed and IL-6 profiles of these regimens were simulated with the SC QSP model. In each dosing scenario, IL-6 profiles were simulated in 300 virtual patients. The cytokine release criterion for regimen selection was that the peak IL-6 concentrations of a SC step-up regimen should be lower than peak IL-6 concentrations observed with the previously tested IV regimens.

Simulation results indicated that the predicted IL-6 profiles were similar with or without split dosing; the former was effectively used in the IV regimen for management of CRS. The predicted highest peak IL-6 value in Week 1 with SC dose of 2 mg was considerably lower than that of IV dosing of 0.7 mg (split 0.2 mg and 0.5 mg over 2 days in Week 1). The predicted highest IL-6 concentration with 2 mg SC was 150 pg/mL and that for 0.7 mg IV was 2,100 pg/mL, indicating a reduction of more than 90%. The predicted IL-6 profiles in Week 4 were similar with 160 mg IV or 400 mg SC doses. Based on the comparison of IL-6 profiles of multiple SC dosing scenarios, SC step-up doses of 2 mg in Week 1, 26 mg in Week 2, and 100 mg in Week 3 were selected, followed by a full dose of 400 mg. The proposed SC regimen is being tested in the clinical study described in Example 5 for safety and efficacy evaluations.

The QSP modeling supported the selection of the odronextamab SC step-up dosing regimen to be evaluated in a clinical study in patients with B-NHL. The selected step-up regimen for SC odronextamab allows adequate step-up to effective therapeutic doses while simplifying and improving overall convenience. The simulations suggest that no split dosing is necessary for SC administration, which may further reduce overall hospital resource burden and requirements for in-patient monitoring.

### Example 12: Odronextamab in Patients with Relapsed/Refractory (R/R) Follicular Lymphoma (FL) Grade 1-3a: Results from a Prespecified Analysis of the Pivotal Phase II Study

Results from a phase 2 study of odronextamab monotherapy in patients with relapsed/refractory (refractory to or relapsed after ≥2 prior lines of therapy, including an anti-CD20 antibody and an alkylator) follicular lymphoma (grades 1-3a), with an ECOG performance status of 0 or 1, are presented below.

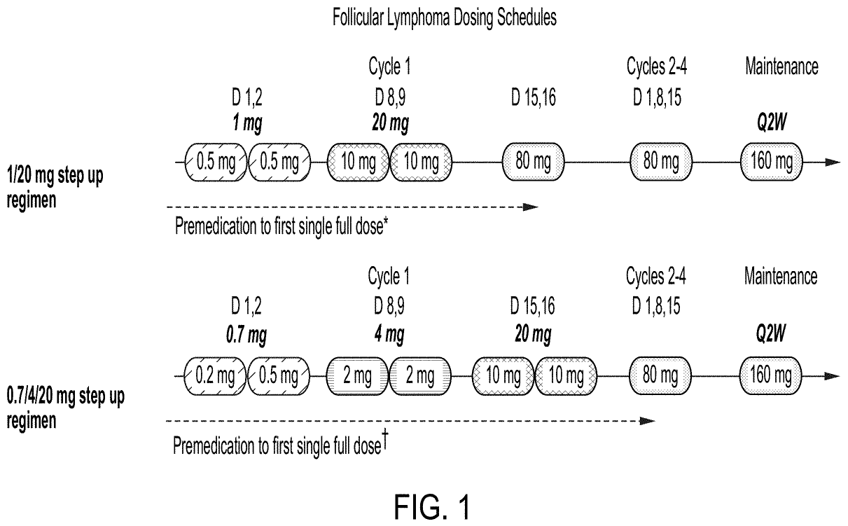

The study was initiated with a 1/20 mg step up dosing regimen that was then modified to a 0.7/4/20 mg step up dosing regimen, as shown in FIG. 1, to further mitigate the risk of cytokine release syndrome (CRS). In the modified dosing regimen, the initial dose (0.7 mg) was split into two dose fractions of 0.2 mg and 0.5 mg administered over two days during week 1 of the dosing regimen, followed by a first intermediate dose (4 mg) split into two dose fractions of 2 mg each administered over two days during week 2 of the dosing regimen, and followed by a second intermediate dose (20 mg) split into two dose fractions of 10 mg each administered over two days during week 3 of the dosing regimen. A full dose (80 mg) was then administered weekly during weeks 4 to 12 of the dosing regimen, followed by every other week dosing of a maintenance dose (160 mg) of odronextamab.

The baseline characteristics of the treated patients (N=131) included 30.5% with prior autologous stem cell transplant (ASCT), 13.7% previously treated with phosphoinositide 3-kinase (PI3K), 13.7% previously treated with an immunomodulatory drug, 71.0% that were refractory to the last line of therapy, 74.8% that were refractory to anti-CD20 antibody therapy, 43.5% that were double refractory to alkylator/anti-CD20 antibody therapy, and 48.1% that showed progression of disease within 24 months of starting first-line therapy.

Disposition of the 131 patients is shown in Table 13, below.

TABLE 13
Disposition of FL Patients

| Cycle 1 step-up regimen (1/20 mg)/(0.7/4/20 mg) | 51.9%/48.1% | |
|---|---|---|
| Median duration of exposure, weeks (range) | 22.1 | (0.4-137.0) |
| Median number of doses (range) | 19 | (1-61) |
| Median number of cycles (range) | 9.1 | (0.1-66.5) |

| | |
|---|---|
| Completed cycle 1 | 95.4% |
| Completed ≥4 cycles | 80.9% |
| Treatment ongoing | 42.7% |
| Treatment discontinued | 57.3% |
| Disease progression | 19.8% |
| Patient or physician decision/withdrawal of consent | 17.6% |
| Adverse event | 9.9% |
| Death | 9.9% |

The objective response rate for the 121 efficacy evaluable patients is shown in Table 14, below, which includes a comparison between the 1/20 step up dosing regimen and the 0.7/4/20 step up dosing regimen. Consistent efficacy was observed at week 12 regardless of the cycle 1 step up regimen, and a majority of patients achieved a complete response.

TABLE 14
Objective Response Rate for FL Patients

| | Independent Central Review |
|---|---|
| Best Overall Response | (N = 121) |
| Objective Response Rate (ORR)* | 81.8% [95% CI; 73.8-88.2%] |
| Complete Response | 75.2% |
| Partial Response | 6.6% |
| Stable Disease | 5.8% |
| Progressive Disease | 4.1% |
| Week 12 Response Assessment | 1/20 Step-Up Dosing Regimen |
| ORR | 72.1% [95% CI; 59.9-82.3%] |
| Complete Response | 61.8% |
| Week 12 Response Assessment | 0.7/4/20 Step-Up Dosing Regimen |
| ORR | 75.5% [95% CI; 61.7-86.2%] |
| Complete Response | 71.7% |

*complete responses + partial responses; CI, confidence interval

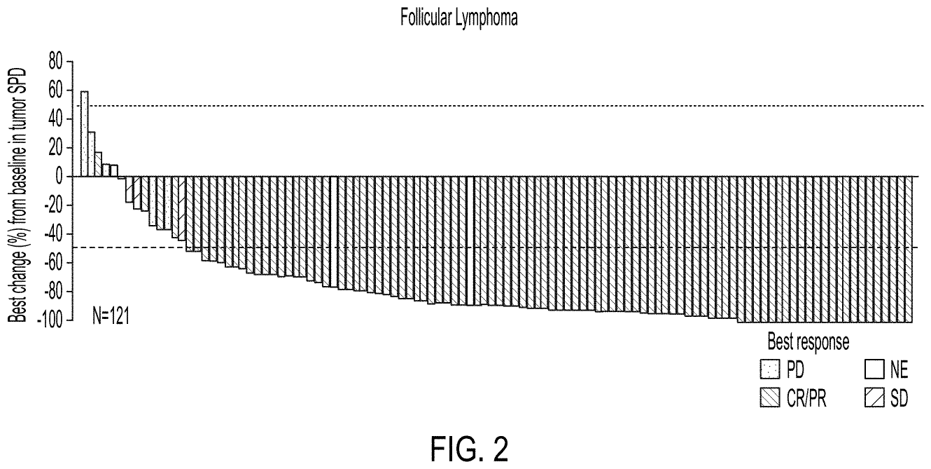

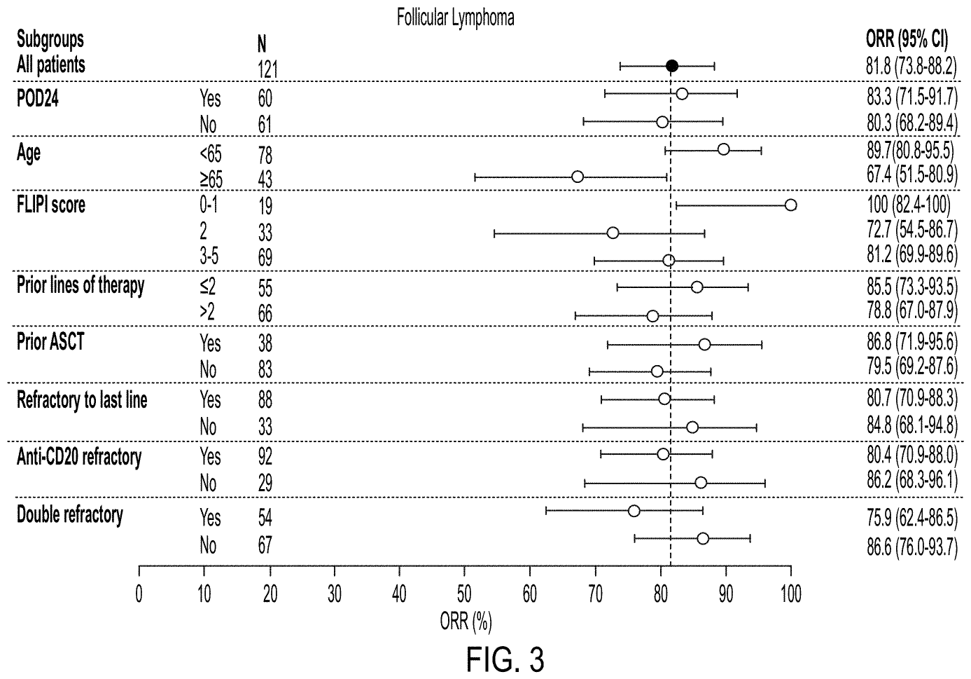

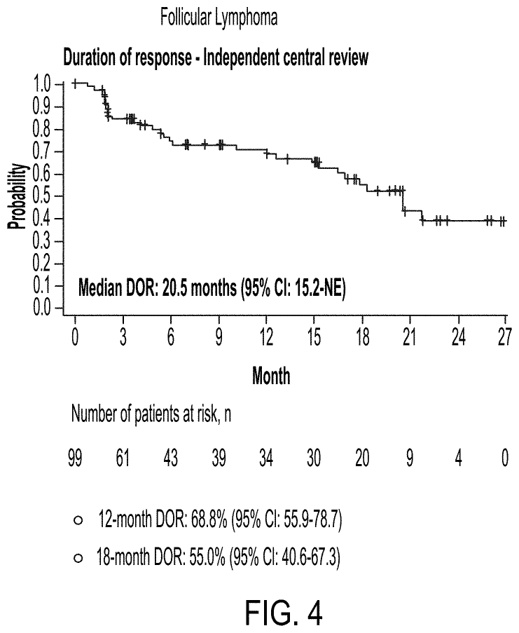

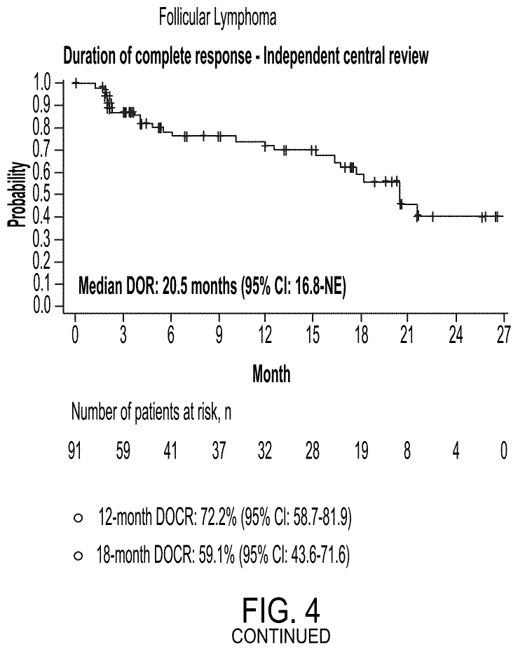

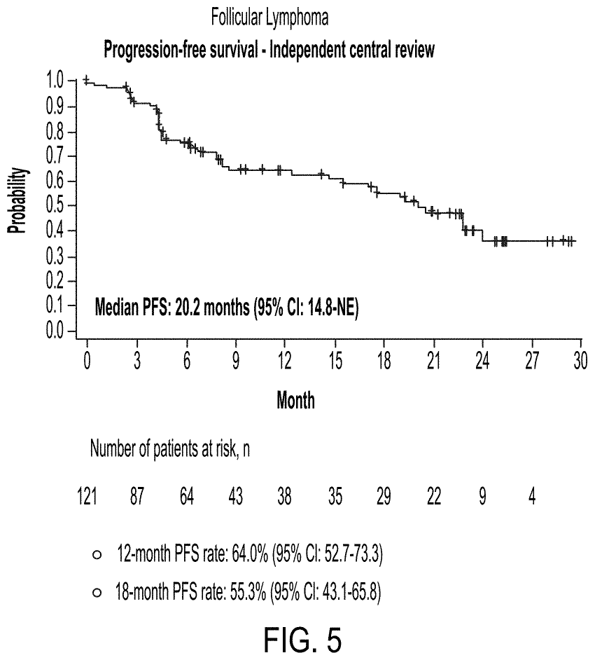

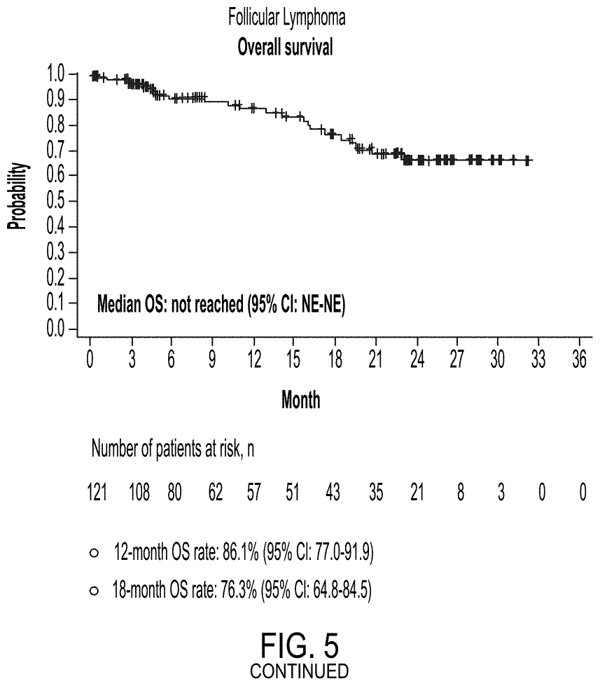

Additional efficacy data for these 121 efficacy evaluable patients is shown in FIGS. 2, 3, 4 and 5, confirming that the majority of relapsed/refractory FL patients has substantial tumor shrinkage (FIG. 2), that there was consistent antitumor activity in high-risk subgroups (FIG. 3), that the observed responses appeared durable (FIG. 4), and that the treatment had a positive impact on progression-free survival and overall survival (FIG. 5).

The safety profile of odronextamab administration in the study, including a comparison of rates/grades of CRS and other adverse events in patients receiving the 1/20 step up dosing regimen or the 0.7/4/20 step up dosing regimen is shown in Tables 15, 16 and 17, below. As noted in Table 16, the 0.7/4/20 step-up dosing regimen reduced the incidence of grade 2 and grade 3 CRS, while approximately half of patients with relapsed/refractory follicular lymphoma had CRS (mostly grade 1). All CRS events resolved within a median of 2 days (range 1-51), and no patients required mechanical ventilation or ICU admission for the management of CRS.

TABLE 15
Odronextamab Safety Profile (N = 131)

| Treatment Emergent AEs, n (%) | All Events | | TRAEs | |
|---|---|---|---|---|
| TEAE | 131 | (100%) | 118 | (90.1%) |
| Grade ≥3 TEAE | 102 | (77.9%) | 73 | (55.7%) |
| Serious AE | 81 | (61.8%) | 53 | (40.5%) |
| Grade 5 TEAE | 17 | (13.0%) | 3 | (2.3%)* |
| Related to COVID-19 | 7 | (4.5%) | — | |
| Leading to treatment discontinuation | 15 | (11.5%) | 10 | (7.6%)** |

*Grade 5 TRAEs: pneumonia, PML, systemic mycosis (n = 1 each)
**TRAEs leading to treatment discontinuation: IRR (n = 3), ALT increase. arthralgia, CRS, epilepsy, PML, tremor, viral bronchitis, weight decrease (n = 1 each)
AE, adverse event; ALT, alanine aminotransferase; CRS, cytokine release syndrome; IRR, infusion related reaction; PML, Progressive multifocal leukoencephalopathy; TEAE, treatment-emergent adverse event; TRAE, treatment-related AE

TABLE 16
Cytokine Release Syndrome

| | | |
|---|---|---|
| | 1/20 Regimen | 0.7/4/20 Regimen |
| n (%) | (N = 68) | (N = 63) |

| | | | | |
|---|---|---|---|---|
| CRS any Grade | 38 | (55.9%) | 36 | (57.1%) |
| Grade 1 | 22 | (32.4%) | 28 | (44.4%) |
| Grade 2 | 12 | (17.6%) | 7 | (11.1%) |
| Grade 3 | 4 | (5.9%) | 1 | (1.6%) |

| | | |
|---|---|---|
| Grade 4 | 0 | 0 |
| Grade 5 | 0 | 0 |

| | | | | |
|---|---|---|---|---|
| Corticosteroids | 11 | (16.2%) | 17 | (27.0%) |
| Tocilizumab | 9 | (13.2%) | 12 | (19.0%) |
| Vasopressors | 4 | (5.9%) | 1 | (1.6%) |

TABLE 17
All Events of Interest

| | 1/20 Regimen | | 0.7/4/20 Regimen | |
|---|---|---|---|---|
| n (%) | (N = 68) | | (N = 63) | |
| ICANS, any grade | 1 | (1.5%) | 0 | |
| Grade ≥3 | 0 | | | |
| Infusion related reaction, any grade | 21 | (30.9%) | 16 | (25.4%) |
| Grade ≥3 | 4 | (5.9%) | 2 | (3.2%) |
| Infection, any grade | 51 | (75.0%) | 35 | (55.6%) |
| Grade ≥3 | 28 | (41.2%) | 14 | (22.2%) |
| Tumor lysis syndrome, any grade | 1 | (1.5%) | 0 | |
| Grade ≥3 | 1 | (1.5%) | | |

ICANS, immune effector cell-associated neurotoxicity syndrome

Overall, these results demonstrate that odronextamab monotherapy in heavily pretreated relapsed/refractory follicular lymphoma patients was efficacious (ORR of 81.8%, CR of 75.2%, and 92% of responders were complete responders, and a durable response with median progression-free survival of 20.2 months). In addition, the odronextamab monotherapy showed a manageable safety profile, especially with the modified 0.7/4/20 step-up dosing regimen.

As of the 18 Sep. 2022 data cut, of the 166 patients treated with the revised step-up regimen of 07/4/20 mg, 86 (51.9%) patients experienced CRS, highest grade of CRS observed was grade 1 in 62 (37.3%) patients, grade 2 in 21 (12.7%) patients and grade 3 in 3 (1.8%) of patients. The revised (0.7/4/20) step-up dosing regimen was successful in reducing further the incidence and severity of CRS events. Incidence of any grade CRS events decreased from 183/300 (61%) patients with the 1/20 regimen to 86/166 (51.8%) patients with the revised regimen and grade 3 or higher CRS events decreased from 8.7% with the 1/20 regimen to 1.8% with the revised regimen. In addition, the overall incidence of grade 2 or higher CRS events decreased from 28.3% with the 1/20 regimen to 14.5% with the revised regimen.

### Example 13: Odronextamab in Patients with Relapsed/Refractory (R/R) Diffuse Large B-Cell Lymphoma (DLBCL): Results from a Prespecified Analysis of the Pivotal Phase II Study

Results from a phase 2 study of odronextamab monotherapy in patients with relapsed/refractory (refractory to or relapsed after ≥2 prior lines of therapy, including an anti-CD20 antibody and an alkylator) diffuse large B-cell lymphoma (per WHO 2016 classification), with an ECOG performance status of 0 or 1, are presented below.

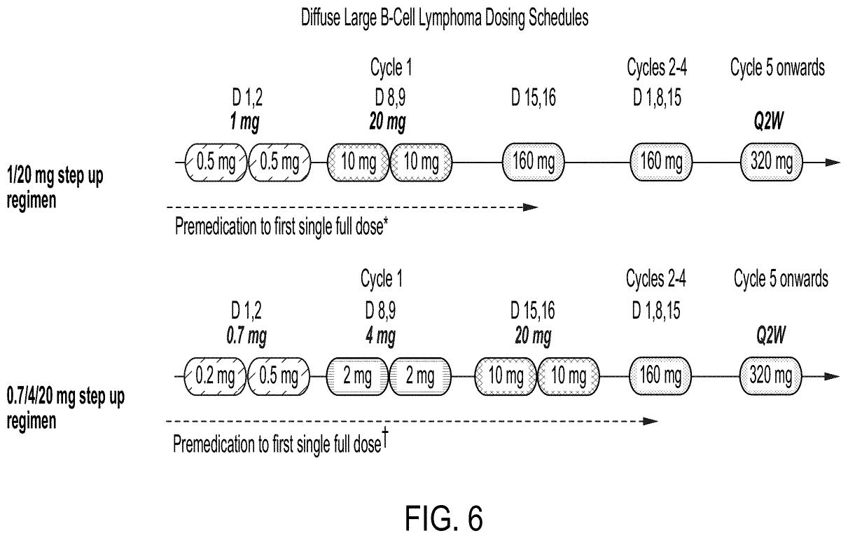

The study was initiated with a 1/20 mg step up dosing regimen that was then modified to a 0.7/4/20 mg step up dosing regimen, as shown in FIG. 6, to further mitigate the risk of cytokine release syndrome (CRS). In the modified dosing regimen, the initial dose (0.7 mg) was split into two dose fractions of 0.2 mg and 0.5 mg administered over two days during week 1 of the dosing regimen, followed by a first intermediate dose (4 mg) split into two dose fractions of 2 mg each administered over two days during week 2 of the dosing regimen, and followed by a second intermediate dose (20 mg) split into two dose fractions of 10 mg each administered over two days during week 3 of the dosing regimen. A full dose (160 mg) was then administered weekly during weeks 4 to 12 of the dosing regimen, followed by every other week dosing of a maintenance dose (320 mg) of odronextamab.

The baseline characteristics of the treated patients (N=140) included 15.7% with prior autologous stem cell transplant (ASCT), 57.1% primary refractory, 90.7% that were refractory to any prior line of therapy, 86.4% that were refractory to the last line of therapy, 78.6% that were refractory to anti-CD20 antibody therapy in any line of therapy, and 65.7% that were double refractory to alkylator/anti-CD20 antibody therapy in any line of therapy.

Disposition of the 140 patients is shown in Table 18, below.

TABLE 18
Disposition of DLBCL Patients

| Cycle 1 step-up regimen (1/20 mg)/(0.7/4/20 mg) | 47.9%/52.1% | |
|---|---|---|
| Median duration of exposure, weeks (range) | 14.9 | (0.9-118.9) |
| Median number of doses (range) | 15 | (1-52) |
| Median number of cycles (range) | 5 | (0-57) |

| | |
|---|---|
| Completed cycle 1 | 91.4% |
| Completed ≥4 cycles | 58.6% |
| Treatment ongoing | 24.3% |
| Treatment discontinued | 75.7% |
| Disease progression | 41.4% |
| Patient or physician decision/withdrawal of consent | 10.7% |
| Adverse event | 9.3% |
| Death | 12.9% |

The objective response rate for the 130 efficacy evaluable patients is shown in Table 19, below, which includes a comparison between the 1/20 step up dosing regimen and the 0.7/4/20 step up dosing regimen. Consistent efficacy was observed at week 12 regardless of the cycle 1 step up regimen, and 62% of responders achieved a complete response.

TABLE 19
Objective Response Rate for DLBCL Patients

| | Independent Central Review |
|---|---|
| Best Overall Response | (N = 130) |
| Objective Response Rate (ORR)* | 49.2% [95% CI; 40.4-58.1%] |
| Complete Response | 30.8% |
| Partial Response | 18.5% |
| Stable Disease | 3.8% |
| Progressive Disease | 22.3% |
| Week 12 Response Assessment | 1/20 Step-Up Dosing Regimen |
| ORR | 46.3% [95% CI; 34.0-58.9%] |
| Complete Response | 26.9% |
| Week 12 Response Assessment | 0.7/4/20 Step-Up Dosing Regimen |
| ORR | 42.9% [95% CI; 30.5-56.0%] |
| Complete Response | 20.6% |

*complete responses + partial responses;
CI, confidence interval

The objective response rate for the 31 efficacy evaluable patients with prior CAR-T therapy is shown in Table 20, below.

TABLE 20
Objective Response Rate for DLBCL Patients with Prior CAR-T

| | Independent Central Review |
|---|---|
| Best Overall Response | (N = 31) |
| Objective Response Rate (ORR)* | 48.4% [95% CI; 30.2-66.9%] |
| Complete Response | 32.3% |
| Partial Response | 16.1% |
| Stable Disease | 6.5% |
| Progressive Disease | 9.7% |

*complete responses + partial responses;
CI, confidence interval;
NE, not evaluable

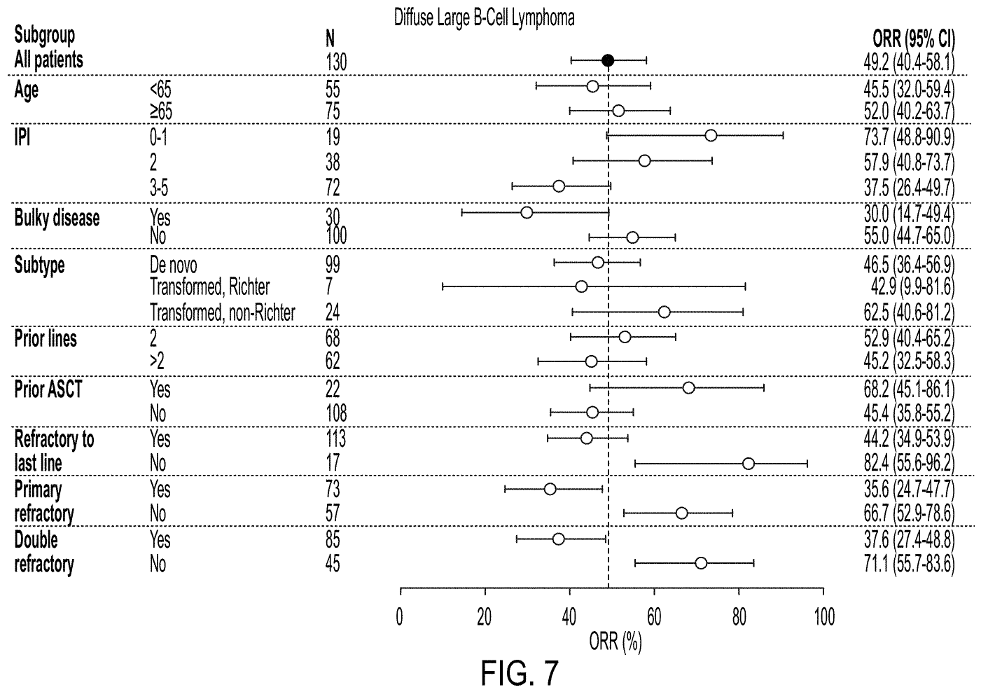

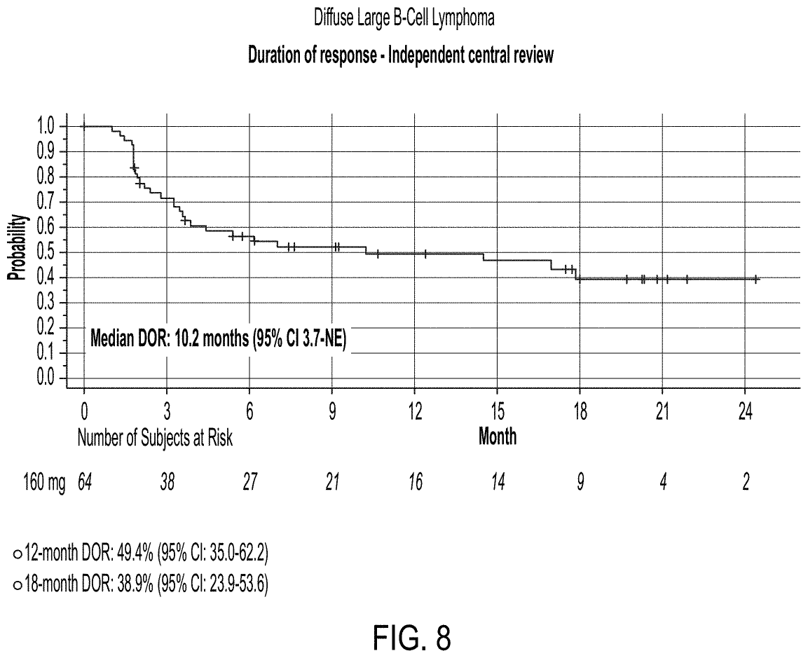

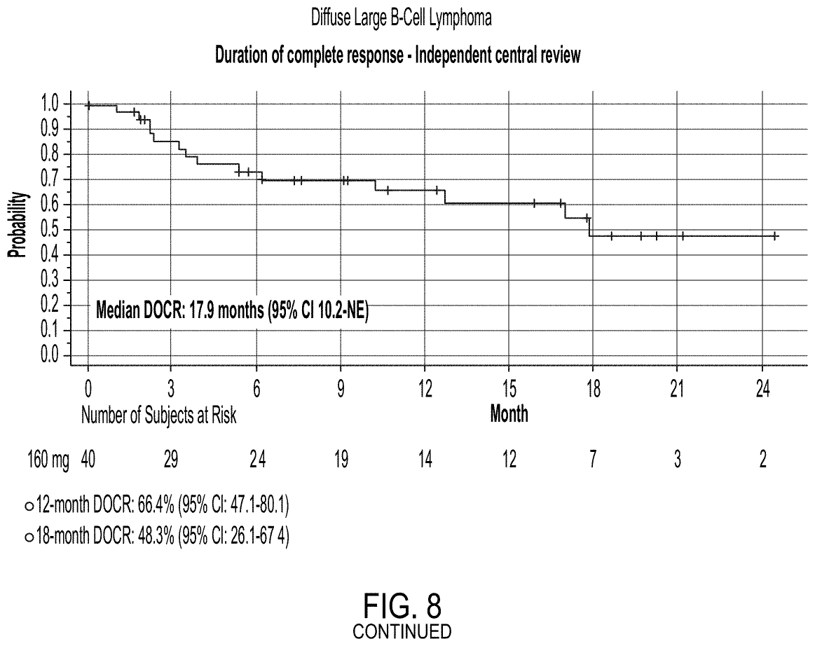

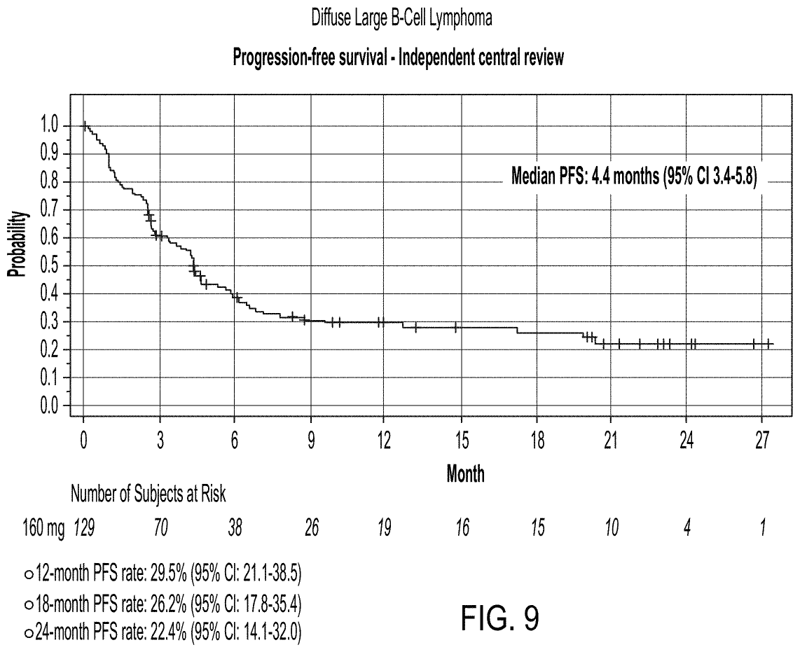

Additional efficacy data for the 130 efficacy evaluable patients is shown in FIGS. 7, 8 and 9, confirming that there was consistent antitumor activity in high-risk subgroups (FIG. 7), that the observed responses appeared durable (FIG. 8), and that the treatment had a positive impact on progression-free survival (FIG. 9).

The safety profile of odronextamab administration in the study, including a comparison of rates/grades of CRS and other adverse events in patients receiving the 1/20 step up dosing regimen or the 0.7/4/20 step up dosing regimen is shown in Tables 21, 22 and 23, below. As noted in Table 22, the 0.7/4/20 step-up dosing regimen reduced the incidence of grade 2 and grade 3 CRS, while approximately half of patients with relapsed/refractory DLBCL had CRS (mostly grade 1). All CRS events resolved within a median of 2 days (range 1-133), and no patients required mechanical ventilation or ICU admission for the management of CRS.

TABLE 21
Odronextamab Safety Profile (N = 140)

| Treatment Emergent AEs, n (%) | All Events | | TRAEs | |
|---|---|---|---|---|
| TEAE | 139 | (99.3%) | 123 | (87.9%) |
| Grade ≥3 TEAE | 110 | (78.6%) | 74 | (52.9%) |
| Serious AE | 85 | (60.7%) | 64 | (45.7%) |
| Grade 5 TEAE | 20 | (14.3%) | 5 | (3.6%)* |
| Related to COVID-19 | 5 | (3.65%) | 1 | (0.7%) |
| Leading to treatment discontinuation | 14 | (10.0%) | 11 | (7.9%)** |

*Grade 5 TRAEs: CMV reactivation, COVID-19, interstitial lung disease, pneumonia, CMV pneumonia, fungal pneumonia, and pseudomonal sepsis (n = 1 each)
**TRAEs leading to treatment discontinuation: CRS, encephalopathy (n = 2 each), CMV reactivation, pneumonia, PJP pneumonia, fungal pneumonia, aphasia, neutrophil count decreased, cough, interstitial lung disease, supraventricular tachycardia, tachycardia, pancreatitis, sclerosing cholangitis, and septic shock (n = 1 each)
AEs per NCI-CTCAE v5.0. CRS per Lee 2019 criteria.
AE, adverse event;
CMV, cytomegalovirus;
CRS, cytokine release syndrome;
IRR, infusion related reaction;
PJP, pneumocystis jirovecii pneumonia
TEAE, treatment-emergent adverse event;
TRAE, treatment-related AE

TABLE 22
Cytokine Release Syndrome

| | | |
|---|---|---|
| | 1/20 Regimen | 0.7/4/20 Regimen |
| n (%) | (N = 67) | (N = 73) |

| | | | | |
|---|---|---|---|---|
| CRS any Grade | 38 | (56.7%) | 39 | (53.4%) |
| Grade 1 | 21 | (31.3%) | 28 | (38.4%) |
| Grade 2 | 12 | (17.9%) | 10 | (13.7%) |
| Grade 3 | 5 | (7.5%) | 1 | (1.4%) |

| | | |
|---|---|---|
| Grade 4 | 0 | 0 |
| Grade 5 | 0 | 0 |

| | | | | |
|---|---|---|---|---|
| Corticosteroids | 13 | (19.4%) | 15 | (20.5%) |
| Tocilizumab | 10 | (14.9%) | 19 | (26.0%) |
| Vasopressors | 5 | (7.5%) | 1 | (1.4%) |

TABLE 23
All Events of Interest

| | 1/20 Regimen | | 0.7/4/20 Regimen | |
|---|---|---|---|---|
| n (%) | (N = 67) | | (N = 73) | |
| ICANS, any grade | 3 | (4.5%) | 1 | (1.4%) |
| Grade ≥3 | 1 | (1.5%)* | 0 | |
| Infusion related reaction, any grade | 16 | (23.9%) | 8 | (11.0%) |
| Grade ≥3 | 0 | | 0 | |
| Infection, any grade | 40 | (59.7%) | 43 | (58.9%) |
| Grade ≥3 | 27 | (40.3%) | 19 | (26.0%) |
| Tumor lysis syndrome, any grade | 1 | (1.5%) | 0 | |
| Grade ≥3 | 1 | (1.5%) | | |

*Grade 3 ICANS event = encephalopathy.
ICANS, immune effector cell-associated neurotoxicity syndrome

Overall, these results demonstrate that odronextamab monotherapy in heavily pretreated relapsed/refractory diffuse large B-Cell lymphoma patients was efficacious (ORR of 49.2%, CR of 30.8%, and a durable response with median duration of complete response of 17.9 months) both prior to and post-CAR-T. In addition, the odronextamab monotherapy showed a manageable safety profile, especially with the modified 0.7/4/20 step-up dosing regimen.

As of 18 Sep. 2022, among the 166 patients treated with the revised step-up regimen of 0.7/4/20 mg, 86 (51.8%) participants experienced CRS. The highest grade of CRS (cytokine release syndrome) observed was Grade 1 in 62 (37.3%) participants, Grade 2 in 21 (12.7%) participants and Grade 3 in 3 (1.8%) participants. The revised step-up dosing regimen was successful in reducing the incidence and severity of CRS events. Incidence of any grade CRS events decreased from 183/300 (61%) participants with the 1/20 regimen to 86/166 (51.8%) participants with the revised regimen, and Grade 3 or higher CRS events decreased from 8.7% with the 1/20 regimen to 1.8% with the revised regimen. The overall incidence of Grade 2 or higher CRS events decreased from 28.3% with the 1/20 regimen to 14.5% with the revised regimen.

### Example 14: Clinical Evaluation of Odronextamab (REGN1979), an Anti-CD20 x Anti-CD3 Bispecific Antibody, Versus Investigator's Choice in Previously Untreated Participants with Follicular Lymphoma

This is an open-label, multicenter, randomized, phase 3 study to compare the efficacy and safety of odronextamab to investigator's choice of chemotherapy (Rituximab-CHOP, Rituximab-CVP, or Rituximab-Bendamustine) for treatment of participants with previously untreated follicular lymphoma. In Part 1 (safety run-in), the intended dose of odronextamab monotherapy to carry forward to Part 2 (randomized phase) will be tested. The efficacy and safety of the recommended dose of odronextamab will be evaluated in Part 2 versus investigator's choice of chemotherapy. All participants will receive 6 cycles of induction. At the end of cycle 6 induction, participants with complete response (CR) or partial response (PR) will continue to the maintenance therapy period with monotherapy treatment with either odronextamab or rituximab, depending on the study arm. Participants who relapse/progress during treatment or are in stable disease (SD) at the end of induction will be withdrawn from the treatment and continue to safety and survival follow-ups.

Participants with high-risk FL (Follicular Lymphoma International Prognostic Index 1 [FLIPI 1] score of 3-5) will be enrolled in the safety lead-in part (from 12-24 patients). Approximately 446 participants will be enrolled and will be randomly assigned in a 1:1 ratio to receive either (A) odronextamab for induction, followed by odronextamab maintenance, or (B) investigator's choice of immuno-chemotherapy, followed by rituximab maintenance. Selection of therapy (CHOP, CVP, or bendamustine) will be according to the investigator's clinical judgement. For participants assigned to receive odronextamab, odronextamab will be given as monotherapy for 6 cycles at the dose selected during Part 1 (see Table 24). At the end of cycle 6, based on week 24 scan assessments, participants with CR or PR will continue treatment with odronextamab monotherapy at a dose of 320 mg Q8W for up to 12 doses or until disease progression, loss to follow-up, or withdrawal of consent, whichever is earlier. For participants assigned to receive rituximab combined with chemotherapy, the treatment will be per standard practice, 6 cycles of induction chemotherapy, followed by up to 12 doses of rituximab monotherapy at Q8W intervals (participants with CR and PR only), or until disease progression, loss to follow-up, or withdrawal of consent, whichever is earlier.

TABLE 24
Odronextamab Dose De-Escalation Schema

| | Odronextamab Dose | | | | | |
|---|---|---|---|---|---|---|
| | | | | | | Q8W |
| Dose | | Intermediate | Intermediate | | | Maintenance |
| Level | Initial dose | dose 1 | dose 2 | Full dose | 2× full dose | dose |
| (DL) | Cycle 1 | | | Cycle 2 to 4 | Cycle 5 & 6 | Cycle 7 to 18 |
| 1 | QW 0.7 mg | QW 4 mg | QW 20 mg | 80 mg | 160 mg | 320 mg |
| | (split 0.2/0.5) | (split 2/2) | (split 10/10) | | | |
| −1 | QW 0.7 mg | QW 4 mg | QW 20 mg | 40 mg | 80 mg | 320 mg |
| | (split 0.2/0.5) | (split 2/2) | (split 10/10) | | | |

The study population will consist of participants of 18 years and older with previously untreated CD20⁺ FL based on World Health Organization (WHO) classification.

Inclusion Criteria: A participant must meet the following criteria to be eligible for inclusion in the study:
1. Have diagnoses of CD20⁺ FL Grade 1-3a, stage II bulky or stage III/IV: Local histopathologic confirmation of the CD20⁺ FL Grade 1 to 3a, must be obtained before study enrollment. Biopsies must have been obtained within 18 months prior to study enrollment. A corresponding tumor biopsy sample should be submitted to the central laboratory. For Part 1 (Safety Run-In) only: FLIP1 score of 3 to 5.
2. Need for treatment as indicated: Presence of ≥1 of the following: B symptoms, large tumor mass (characterized by lymphomas with a diameter >3 cm in 3 or more regions or by a lymphoma with a diameter >6 cm in 1 region), and/or presence of lymphoma-related complications.
3. Have measurable disease on cross-sectional imaging documented by diagnostic imaging CT or MRI (measurable disease is defined as at least 1 bidimensionally measurable nodal lesion of >1.5 cm or extranodal disease of >1 cm in the greatest transverse diameter (GTD) regardless of the short axis diameter).
4. Eastern Cooperative Oncology Group (ECOG) performance status of 0-2.
5. Age ³ 18 years
6. Adequate bone marrow function as documented by:
   a. Platelet count ³ 50×10⁹/L. A participant may not have received platelet transfusion therapy within 7 days prior to first dose of odronextamab in order to meet the platelet eligibility criterion.
   b. Hemoglobin ³ 9.0 g/dL
   c. Absolute neutrophil count (ANC)≥³ 1.0×109/L. A participant may not have received G-CSF within 2 days prior to the first dose of odronextamab in order to meet the ANC eligibility criterion.
   d. Participants with bone marrow involvement or splenic sequestration should meet the following hematologic parameters:
      Platelet count ³ 25×10⁹/L. A participant may not have received platelet transfusion therapy within ≥3 days prior to first dose of odronextamab in order to meet the platelet eligibility criterion.
      Hemoglobin ³ 7.0 g/dL
      ANC≥0.5×10⁹/L. A participant may not have received G-CSF within 2 days prior to first dose of odronextamab in order to meet the ANC eligibility criterion.
7 Participants with adequate hepatic function:
   a. Total bilirubin ≤1.5×upper limit of normal (ULN) (≤3×ULN if attributed to lymphoma infiltration of liver).
   b. Alanine aminotransferase (ALT) and aspartate aminotransferase (AST)≤2.5×ULN (≤5×ULN if attributed to lymphoma infiltration of liver).
   c. Alkaline phosphatase (ALP)≤2.5×ULN (≤5×ULN if attributed to lymphoma infiltration of liver).
   NOTES:
      Irrespective of the presence of lymphoma infiltration of the liver, a participant with an AST>2.5×ULN and/or ALT>2.5×ULN concurrent with a total bilirubin >1.5×ULN will be excluded.
      Participants with known Gilbert syndrome will be excluded if the total bilirubin value is >4×ULN for the local general population.
8. Calculated creatinine clearance by Cockcroft-Gault formula ³ 50 mL/min.
   NOTE: Participants with a calculated creatinine clearance <50 mL/min may be considered for enrollment if a measured creatinine clearance (based on 24-hour urine collection or other reliable method) is ≥50 mL/min.
9. Ability to understand the purpose and risks of the study and provide signed and dated informed consent and authorization to use protected health information by study participant or legally acceptable representative (in accordance with national and local participant privacy regulations).
10. Willing and able to comply with clinic visits and study-related procedures.
11. Able to understand and complete study-related questionnaires.

Exclusion Criteria: A participant who meets any of the following criteria will be excluded from the study:
1. Participants with central nervous system (CNS) lymphoma or leptomeningeal lymphoma.
   a. Primary CNS lymphoma or known involvement by non-primary CNS lymphoma (even if treated into complete remission).
   b. Suspected CNS involvement by lymphoma must be evaluated by CNS imaging (MRI or CT) and by lumbar puncture as appropriate.
2. Participants with histological evidence of transformation to a high-grade or diffuse large B-cell lymphoma.
3. Participants with Waldenström macroglobulinemia (WM, lymphoplasmacytic lymphoma), Grade 3b follicular lymphoma, chronic lymphocytic leukemia, or small lymphocytic lymphoma.
4. Treatment with any systemic anti-lymphoma therapy.
5. Recent major surgery (within 4 weeks prior to the start of assigned study treatment).
6. Standard radiotherapy within 14 days of first administration of assigned study treatment.
7. History of solid organ transplantation.
8. Continuous systemic corticosteroid treatment with more than 10 mg per day of prednisone or anti-inflammatory equivalent within 72 hours of start of study drug.
9. A malignancy other than NHL unless the participant is adequately and definitively treated and is cancer free for at least 3 years with the exception of localized prostate cancer treated with hormone therapy or local radiotherapy (i.e. pellets), cervical carcinoma in situ, breast cancer in situ, or nonmelanoma skin cancer that was definitively treated.
10. Any other significant active disease or medical condition that could interfere with the conduct of the study or put the participant at significant risk, including but not limited to significant cardiovascular disease (eg, New York Heart Association Class III or IV cardiac disease, myocardial infarction within the previous 6 months, unstable arrhythmias, or unstable angina); significant pulmonary disease (eg, obstructive pulmonary disease and history of symptomatic bronchospasm); or gastrointestinal, hepatic, renal, endocrine, hematologic, autoimmune, psychiatric, or neurologic disorder.
11. History of or current relevant CNS pathology, such as epilepsy, seizure, paresis, aphasia, apoplexy, severe brain injury, cerebellar disease, organic brain syndrome, psychosis, inflammatory lesions, and/or vasculitis.
12. Vaccination within 28 days prior to first study drug administration with a vector that has replicative potential.
13. Cardiac ejection fraction <50% by echocardiogram or multigated acquisition (MUGA) scan.
14. Pregnant or breastfeeding women.
15. Women of childbearing potential (WOCBP)* or men who are unwilling to practice highly effective contraception prior to the initial dose/start of the first treatment, during the study, and for at least 6 months after the last dose. Sperm donation is prohibited during the study and for 6 months after the last dose of study drug. Highly effective contraceptive measures include:
   a. stable use of combined (estrogen and progestogen containing) hormonal contraception (oral, intravaginal, transdermal) or progestogen-only hormonal contraception (oral, injectable, implantable) associated with inhibition of ovulation initiated 2 or more menstrual cycles prior to screening;
   b. intrauterine device (IUD); intrauterine hormone-releasing system (IUS);
   c. bilateral tubal occlusion/ligation;
   d. vasectomized partner (provided that the male vasectomized partner is the sole sexual partner of the WOCBP study participant and that the vasectomized partner has obtained medical assessment of surgical success for the procedure); and/or
   e. sexual abstinence.
16. History of clinically significant cardiovascular, respiratory, hepatic, renal, gastrointestinal, endocrine, hematological, psychiatric, or neurological disease, as assessed by the investigator, that may confound the results of the study or poses an additional risk to the participant by study participation.
17. Has a history of tuberculosis or systemic fungal diseases that has been active within 6 months.
18. Infections:
   Infection requiring hospitalization or treatment with IV anti-infectives within 2 weeks of first administration of assigned study treatment. There should be evidence that the infection has cleared or is well controlled by start of study therapy.
   Evidence of any active infection (bacterial, viral, fungal, mycobacterial, parasitic, or other) at study enrollment or within 2 weeks of study enrollment, if requiring ongoing treatment and/or has the potential to cause disseminated disease or severe infection upon immunosuppression.
   Active COVID-19 infection defined as PCR+
   Uncontrolled infection with human immunodeficiency virus (HIV), hepatitis B (HBV), or hepatitis C (HCV).
   Cytomegalovirus (CMV) infection as noted by detectable levels on peripheral blood PCR assay. Participants who show detectable levels of CMV at screening will need to be treated with appropriate antiviral therapy and demonstrate at least 2 undetectable levels of CMV by PCR assay (at least 7 days apart) before being re-considered for eligibility.
   NOTE: Participants with HIV who have controlled infection (undetectable viral load and CD4 count above 350 cells/μL either spontaneously or on a stable antiviral regimen) are permitted.
   NOTE: Participants who are hepatitis B surface antigen positive or who are hepatitis B core antibody positive should undergo evaluation by a specialist and be considered for antiviral prophylaxis, before they are permitted onto study.
   NOTE: Participants who are hepatitis C virus antibody positive (HCV Ab+) who have controlled infection (undetectable HCV RNA by PCR either spontaneously or in response to a successful prior course of anti-HCV therapy) are permitted.
19. Allergy/hypersensitivity:
   a. History of severe allergic reaction attributed to compounds with a similar chemical or biologic composition as that of the study drug or excipient.
   b. Known hypersensitivity to both allopurinol and rasburicase.
20. Participated in any clinical research study evaluating another investigational drug including biologics or therapy, including specific immunotherapy, within 90 days or at least 5 half-lives (whichever is longer) of an investigational biologic drug, or at least 4 weeks for other investigational drug, prior to the screening visit.
21. Participants who are committed to an institution by virtue of an order issued either by the judicial or the administrative authorities.

The study interventions administered in this study are shown in Table 25, below.

TABLE 25
Study Interventions Administered

| | Arm 1 | Arm 2 | | | | | |
|---|---|---|---|---|---|---|---|
| | Intervention Name | | | | | | |
| | Odronextamab | Rituximab | CP | DX | VC | PN | BD |
| Dosage Level(s) | See Table 26 | 375 mg/m² | 750 mg/m² | 50 mg/m² | 1.4 mg/m² | 100 mg | 90 mg/m² |
| Route of | IV infusion | IV infusion | IV | IV | IV | PO | IV |
| Administration | | | | | | | |

CP—cyclophosphamide;
DX—doxorubicin;
VC—vincristine;
PN—prednisone;
BD—bendamustine

Odronextamab dose administered is a flat dose and not dependent on participant weight or body surface area. During the induction treatment period, odronextamab will be administered IV with step-up dosing in cycle 1 to mitigate the risk for CRS. Cycle 1 will consist of an initial dose of 0.7 mg (split as 0.2 mg on cycle 1 day 1 and 0.5 mg on cycle 1 day 2), an intermediate dose 1 of 4 mg (split as 2 mg on cycle 1 day 8 and 2 mg on cycle 1 day 9), and an intermediate dose 2 of 20 mg (split as 10 mg on cycle 1 day 15 and 10 mg on cycle 1 day 16). From cycle 2 to cycle 4, odronextamab will be administered IV on days 1, 8, and 15 at 80 mg, and from cycle 5 and cycle 6, odronextamab will be administered IV at 160 mg on day 8 in cycle 5 and days 1 and 15 in cycle 6 (Table 26). During the monotherapy maintenance treatment period, odronextamab will be administered IV Q8W at 320 mg (Table 26). The first dose of odronextamab (320 mg) during maintenance will be administered 6 weeks after the last dose (160 mg) given during induction on cycle 6 day 15.

TABLE 26
Odronextamab Administration
Odronextamab Dose Regimen

| | | | | | | | | Cycle 6 | |
|---|---|---|---|---|---|---|---|---|---|
| | | Cycle 1 | Cycle 1 | Cycle 1 | Cycle 1 | Cycle 2-4 | Cycle 5 | Day 1 | M1 to M12 |
| Cycle 1 | Cycle 1 | Day 8 | Day 9 | Day 15 | Day 16 | Day 1, 8 | Day 8 | and 15 | Day 1 |
| Day 1 | Day 2 | Intermediate 1 | | Intermediate 2 | | and 15 | 2× full | 2× full | Maintenance |
| Initial (0.7 mg) | | (4 mg) | | (20 mg) | | Full dose | dose | dose | Q8W |
| 0.2 mg | 0.5 mg | 2 mg | 2 mg | 10 mg | 10 mg | 80 mg | 160 mg | 160 mg | 320 mg |

For the initial dose, intermediate dose 1, and intermediate dose 2, the treatment will be split into 2 separate infusions given on 2 separate days, which are preferably consecutive but no more than 3 days apart. Each of the split infusions during cycle 1 and the first full dose QW infusion (cycle 2 day 1) should occur over 4 hours. Subsequent treatments may be administered as a single infusion or as 2 separate infusions and may be administered over at least 1 hour depending on tolerability.

Premedications to mitigate the risk and reduce the severity of CRS are detailed in Example 3. CRS toxicity grading is an shown in, e.g., Table 6.

For this study, use of rituximab or biosimilars is permitted. Rituximab must be administered according to institutional guidelines or according to the instructions in the product package insert. Rituximab will be administered IV on day 1 of each cycle in a dose of 375 mg/m². The frequency and duration of rituximab will be Q3W in combination with induction chemotherapy (CHOP/CVP) for 6 cycles of 21 days each and Q4W with induction bendamustine for 6 cycles of 28 days each (induction treatment phase). Upon completion of the combination treatment period or early termination of chemotherapy, rituximab will be continued as monotherapy Q8W for up to 12 maintenance doses (if participant has CR or PR), unless the participant discontinues early due to toxicity, progressive disease, or start of subsequent lymphoma therapy or due to discretionary reasons (participant or investigator). Rituximab and chemotherapy administration is described in Table 27. For details about CHOP/CVP and bendamustine, including formulation and administration, refer to the product package insert. Eight weeks after the dose on cycle 6 day 1, participants (who had CR or PR) will start the maintenance treatment period of rituximab monotherapy on a Q8W schedule.

TABLE 27
CHOP/CVP and Bendamustine Dose

| Investigator's | | | | | | Cycle Length |
|---|---|---|---|---|---|---|
| Choice Arm | Drug | Dose | | Route | Days | (Days)/# of cycles |
| CHOP | Cyclophosphamide | 750 | mg/m² | IV | 1 | 21 days/ |
| | | | | | | 6 cycles |
| | Doxorubicin | 50 | mg/m² | IV | 1 | 21 days/ |
| | Vincristine | 1.4 mg/m² | | IV | 1 | 6 cycles |
| | | (max 2 mg) | | | | |
| | Prednisone | 100 | mg | PO | 1-5 | |
| CVP | Cyclophosphamide | 750 | mg/m² | IV | 1 | 21 days/ |
| | | | | | | 6 cycles |
| | Vincristine | 1.4 mg/m² | | IV | 1 | 21 days/ |
| | | (max 2 mg) | | | | 6 cycles |
| | Prednisone | 100 | mg | PO | 1-5 | |
| Bendamustine | Bendamustine | 90 | mg/m² | IV | 1-2 | 28 days/ |
| | | | | | | 6 cycles |

### Example 15: Clinical Evaluation of Odronextamab (REGN1979), an Anti-CD20 x Anti-CD3 Bispecific Antibody, in Combination with CHOP (O-CHOP) Versus Rituximab in Combination with CHOP (R-CHOP) in Previously Untreated Participants with Diffuse Large B-Cell Lymphoma

This is a phase 3, open-label, randomized, multicenter study of odronextamab in combination with CHOP (O-CHOP) compared to rituximab in combination with CHOP (R-CHOP) for previously untreated participants with DLBCL with an International Prognostic Index (IPI) score of 2 or more. The study consists of 2 Parts: Part 1 of the study is a safety run-in to assess the safety and tolerability of O-CHOP and determine the intended dose regimen of O-CHOP for Part 2; Part 2 is the randomized part of the study, evaluating the efficacy and safety of O-CHOP in comparison to R-CHOP.

All participants will receive only odronextamab (no rituximab) in Part 1. A maximum of 48 participants will be enrolled and treated at the lower Dose Level 1 (DL1) and escalated to DL2 as per Table 28. Dose-limiting toxicities (DLTs) and the tolerability of O-CHOP will also be assessed. Table 28 provides the dosing schema of odronextamab for combination (with CHOP) in Part 1 (Safety Run-in). Dosing level will commence with 80 mg odronextamab full dose (DL 1). CHOP doses remain the same across dose levels; CHOP begins on cycle 1 day 1 (C1D1) for all DLs. CHOP is given every 21 days for a total of 6 cycles (1 cycle=21 days). Odronextamab will commence on C1D8 for all DLs.

TABLE 28
Odronextamab Dose Escalation Schema

| | | | DL 1 | DL 2 |
|---|---|---|---|---|
| | | | Odronextamab | Odronextamab |
| Cycle | Day | Dosing Schema | Dose (mg) | Dose (mg) |
| C1 | D 1 | N/A | N/a | N/A |
| | D 8 | Initial Dose | 0.2 | 0.2 |
| | D 9 | | 0.5 | 0.5 |
| | D 15 | Intermediate Dose 1 | 2 | 2 |
| | D 16 | | 2 | 2 |
| C2 | D 1 | Intermediate Dose 2 | 10 | 10 |
| | D 2 | | 10 | 10 |
| | D 8 | QW | 80 | 160 |
| | D 15 | | 80 | 160 |
| C3 | D 1 | | 80 | 160 |
| | D 8 | QW | 80 | 160 |
| | D 15 | | 80 | 160 |
| C4 | D 1 | | 80 | 160 |
| | D 8 | | 80 | 160 |
| | D 15 | | 80 | 160 |
| C5 | D 1 | | 80 | 160 |
| | D 8 | Q2W | 160 | 320 |
| C6 | D 1 | | 160 | 320 |
| | D 15 | | 160 | 320 |

C = cycle;
D = day;
QW = weekly;
Q2W = every 2 weeks
Note:
If DL1 is intolerable, data will be evaluated to consider lower doses. Doses of chemotherapy agents do not change across dose levels.

In Part 2, approximately 840 participants will be enrolled and randomly assigned in a 1:1 ratio to receive either O-CHOP or R-CHOP. At least 546 participants will be enrolled with an IPI score ≥3. There will be no maintenance therapy in either arm. No crossover between the arms will be allowed. Randomization will be stratified according to International Prognostic Index (2 vs 3 vs 4 to 5) and age (<65 vs ≥65 years old). Study treatment will begin within 5 days of randomization, unless otherwise permitted by medical monitor. For participants assigned to receive O-CHOP, odronextamab will be given in combination with 6 cycles of induction CHOP. For participants assigned to receive R-CHOP, the treatment will be per standard practice, i.e., 6 cycles of induction CHOP.

The study population will consist of participants 18 years and older with intermediate to high risk previously untreated diffuse large B-cell lymphoma.

Inclusion Criteria: A participant must meet the following criteria to be eligible for inclusion in the study:
1. Previously untreated participants for lymphoma with *documented CD20+ DLBCL including one of the following diagnoses as per 2016 WHO classification of lymphoid neoplasms:
   a. **DLBCL, not otherwise specified (NOS) including germinal center B-cell type, activated B-cell type.
   b. High grade B-cell neoplasm, with MYC, bcl-2 and/or bcl-6 rearrangements For Part 1 only: participants will need to have at least one of the high-risk features to be eligible. High risk features include, but are not limited to, IPI score 3 to 5, cell of origin (non-GCB type), double-hit or triple-hit lymphoma (classified as high-grade B cell neoplasm, with MYC, bcl-2 and/or bcl-6 rearrangements according to 2016 WHO classifications of lymphoid neoplasms), TP53 mutations, CDKN2A loss, etc (Sehn and Salles, 2021). *Local histopathologic confirmation of the DLBCL must be obtained before study enrollment. Biopsies must have been obtained within 3 months prior to study enrollment. Availability of tumor tissue for submission to central laboratory is required for study enrollment.**De novo or histologically transformed from a diagnosis of follicular lymphoma
2. Have measurable disease with at least one nodal lesion with LDi greater than 1.5 cm or at least one extranodal lesion with LDi greater than 1.0 cm, documented by diagnostic imaging (computed tomography [CT] or magnetic resonance imaging [MRI]).
3. Age ≥18 years
4. Eastern Cooperative Oncology Group (ECOG) performance status ≤2
5. Life expectancy ≥12 months
6. International Prognostic Index (IPI)≥2
7. Adequate hematologic function, as measured by:
   a. Platelet count ≥75×109/L. A participant may not have received platelet transfusion within 7 days of first dose of the assigned study treatment in order to meet this eligibility requirements
   b. Absolute neutrophil count (ANC)≥1.0×109/L. A participant may not have received granulocyte colony stimulating factor (G-CSF) within 2 days of first dose of the assigned study treatment in order to meet this eligibility requirement
   c. Hemoglobin level ≥9 g/dL
NOTE: Participants with cell counts below thresholds listed above may be considered for enrollment if, in the opinion of the investigator, the reason is believed to be due to bone marrow infiltration or splenic sequestration by the underlying disease.
   Participants with bone marrow involvement or splenic sequestration should meet the following hematologic parameters:
      a. Platelet count ≥25×109/L. A participant may not have received platelet transfusion therapy within 3 days prior to first dose of study treatment in order to meet the platelet eligibility criterion.
      b. Absolute neutrophil count (ANC)≥0.5×109/L. A participant may not have received granulocyte colony stimulating factor within 2 days prior to first dose of study treatment in order to meet the ANC eligibility criterion.
      c. Hemoglobin ≥9.0 g/dL
8. Adequate organ function, as documented by:
   a. Cardiac ejection fraction >50% by echocardiogram or multigated acquisition (MUGA) scan
   b. Total bilirubin ≤1.5×upper limit of normal (ULN) (≤3×ULN if attributed to lymphoma infiltration of liver)
   c. Alanine aminotransferase (ALT) and aspartate aminotransferase (AST)≤2.5×ULN (≤5×ULN if attributed to lymphoma infiltration of liver)
   d. Alkaline phosphatase (ALP)≤2.5×ULN (≤5×ULN if attributed to lymphoma infiltration of liver)
   e. Serum creatinine ≤1.5×ULN, or calculated creatinine clearance by Cockcroft Gault formula ≥50 mL/min
   NOTE: Irrespective of the presence of lymphoma infiltration of the liver, a participant with an AST>3×ULN and/or ALT>3×ULN concurrent with a total bilirubin >1.5×ULN will be excluded.
   NOTE: Participants with known Gilbert syndrome will be excluded if the total bilirubin value is >4×ULN.
   NOTE: Participants with a calculated creatinine clearance <50 mL/min may be considered for enrollment if a measured creatinine clearance (based on 24-hour urine collection or other reliable method) is ≥50 mL/min.
9. Willing and able to comply with clinic visits and study-related procedures.
10. Provide informed consent signed by study participant or legally acceptable representative.
11. Able to understand and complete study-related questionnaires with physical assistance, if required.

Exclusion Criteria: A participant who meets any of the following criteria will be excluded from the study:
1. Primary central nervous system (CNS) lymphoma or known involvement by non-primary CNS NHL
2. History of or current relevant CNS pathology, such as:
   a. epilepsy, seizure, paresis, aphasia, apoplexy, severe brain injury, cerebellar disease, organic brain syndrome, psychosis, or
   b. evidence for presence of inflammatory lesions and/or vasculitis on cerebral MRI
3. Peripheral neuropathy Grade
4. Another active malignancy (aside from B-cell NHL) in the past 5 years, with the following exceptions: non-melanoma skin cancer that has undergone potentially curative therapy, in situ cervical carcinoma, or any other tumor that has been deemed to be effectively treated with definitive local control and with curative intent
5. Any other significant active disease or medical condition that could interfere with the conduct of the study or put the participant at significant risk, including but not limited to significant cardiovascular disease (eg, New York Heart Association Class III or IV cardiac disease, myocardial infarction within the previous 6 months, unstable arrhythmias, or unstable angina) or significant pulmonary disease (eg, obstructive pulmonary disease and history of symptomatic bronchospasm)
6. Treatment with any systemic anti-lymphoma therapy
7 Any investigational therapy within 28 days or 5 half-lives of the drug, whichever is shorter, prior to the start of study treatment
8. Recent major surgery (within 4 weeks prior to the start of study treatment)
9. Standard radiotherapy within 14 days of first administration of study treatment
10. Prior organ transplantation
11. Allergy/hypersensitivity:
   a. Known hypersensitivity to both allopurinol and rasburicase
   b. History of allergic reactions or hypersensitivity attributed to compounds of similar chemical or biological components included in CHOP or rituximab
12. Infections:
   a. Evidence of any active infection (bacterial, viral, fungal, mycobacterial, parasitic or other) at study enrollment or within 2 weeks of study enrollment, if requiring ongoing treatment and/or has the potential to cause disseminated disease or severe infection upon immunosuppression. There should be evidence that the infection has cleared or is well controlled by start of study therapy.
   b. Active COVID-19 infection
   c. Uncontrolled infection with human immunodeficiency virus (HIV), hepatitis B virus (HBV), or hepatitis C virus (HCV)
   d. Cytomegalovirus (CMV) infection as noted by detectable levels on peripheral blood polymerase chain reaction (PCR) assay. Patients who show detectable levels of CMV at screening will need to be treated with appropriate antiviral therapy and demonstrate at least 2 undetectable levels of CMV by PCR assay (at least 7 days apart) before being re-considered for eligibility.
   NOTE: Patients with HIV who have controlled infection (undetectable viral load and CD4 count above 350 cells/μL either spontaneously or on a stable antiviral regimen) are permitted
   NOTE: Patients who are hepatitis B surface antigen positive or who are hepatitis B core antibody positive should undergo evaluation by a specialist and be considered to have controlled infection (serum hepatitis B virus DNA PCR that is below the limit of detection AND receiving anti-viral therapy for hepatitis B) before they are permitted onto study
   NOTE: Patients who are HCV antibody positive who have controlled infection (undetectable HCV RNA by PCR either spontaneously or in response to a successful prior course of anti-HCV therapy) are permitted
13. Administration of live vaccination within 28 days of study first dose (COVID-19 vaccine can be administered no less than one week prior to first dose)
14. Participants who are committed to an institution by an order issued either by the judicial or the administrative authorities (for sites in Germany only)
15. Members of the clinical site study team and/or his/her immediate family, unless prior approval granted by the Sponsor.
16. Pregnant or breastfeeding women.
17. Women of childbearing potential (WOCBP)* or men who are unwilling to practice highly effective contraception prior to the initial dose/start of the first treatment, during the study, and for at least 6 months after the last dose. Sperm donation is prohibited during the study and for 6 months after the last dose of the assigned study treatment. Highly effective contraceptive measures include:
   a. stable use of combined (estrogen and progestogen containing) hormonal contraception (oral, intravaginal, transdermal) or progestogen-only hormonal contraception (oral, injectable, implantable) associated with inhibition of ovulation initiated 2 or more menstrual cycles prior to screening
   b. intrauterine device (IUD); intrauterine hormone-releasing system (IUS)
   c. bilateral tubal ligation/occlusion
   d. vasectomized partner (provided that the male vasectomized partner is the sole sexual partner of the WOCBP study participant and that the vasectomized partner has obtained medical assessment of surgical success for the procedure)
   e. sexual abstinence

The study interventions administered in this study are shown in Table 25, below.

TABLE 29
Study Interventions Administered

| | Arm 1: | Arm 2: | | | | |
|---|---|---|---|---|---|---|
| | O-CHOP | R-CHOP | CHOP | | | |
| | Intervention Name | | | | | |
| | Odronextamab | Rituximab | CP | DX | VC | PN |
| Dosage Level (s) | See Table 30 | 375 mg/m² | 750 mg/m² | 50 mg/m² | 1.4 mg/m² | 100 mg |
| Route of | IV infusion | IV infusion | IV | IV | IV | PO |
| Administration | | | | | | |

CP—cyclophosphamide;
DX—doxorubicin;
VC—vincristine;
PN—prednisone

Odronextamab will be administered in combination with CHOP (O-CHOP) for 6 cycles of 21 days. CHOP will be dosed on C1D1 and odronextamab dosing will begin on C1D8 to decrease the risk for CRS. During the induction treatment period, odronextamab is administered intravenously with step-up dosing in cycles 1 and 2 to mitigate the risk of CRS. Step-up dosing consists of an initial dose of 0.7 mg (split as 0.2 mg on C1D8 and 0.5 mg on C1D9), an intermediate dose 1 of 4 mg (split as 2 mg on C1D15 and 2 mg on C1D16), followed by intermediate dose 2 of 20 mg (split as 10 mg on C2D1 and 10 mg on C2D2). From C2D8 up to C5D1, odronextamab is administered intravenously weekly on days 1 (except cycle 2), 8 and 15 at 80 or 160 mg. From C5D8, Q2W administration of odronextamab will commence with doses on C5D8 and C6D1 and C6D15. See Table 30.

TABLE 30
Odronextamab Administration
Odronextamab Dose Regimen

| | | | | | | Weekly dose (QW) | Every 2 |
|---|---|---|---|---|---|---|---|
| | | | | | | Cycle 2: Day 8, | Weeks dose |
| | | Intermediate 1 | | Intermediate 2 | | Day 15 | (Q2W) |
| Initial (0.7 mg) | | (4 mg) | | (20 mg) | | Cycle 3 to 4: | Cycle 5: Day 8 |
| Cycle 1 | Cycle 1 | Cycle 1 | Cycle 1 | Cycle 2 | Cycle 2 | Day 1, Day 8 and 15 | Cycle 6: Days |
| Day 8 | Day 9 | Day 15 | Day 16 | Day 1 | Day 2 | Cycle 5: Day 1 | 1 and 15 |
| 0.2 mg | 0.5 mg | 2 mg | 2 mg | 10 mg | 10 mg | 80 or 160 mg | 160 or 320 mg |

For the initial dose, intermediate dose 1 and intermediate dose 2, the treatment will be split into 2 separate infusions given on 2 separate days which are preferably consecutive, but no more than 3 days apart. Each of the split infusion during cycle 1 and cycle 2 and the first full dose QW infusion (C2D8) should occur over 4 hours. Subsequent treatments may be administered as a single infusion or as 2 separate infusions over at least 1 hour depending on tolerability. Administration of the C2D8 full dose will only proceed if the initial (0.7 mg) and intermediate dose 1 (4 mg) and intermediate dose 2 (20 mg) were received and tolerated (participants who received >90% of the intended dose are considered to have received the full dose).

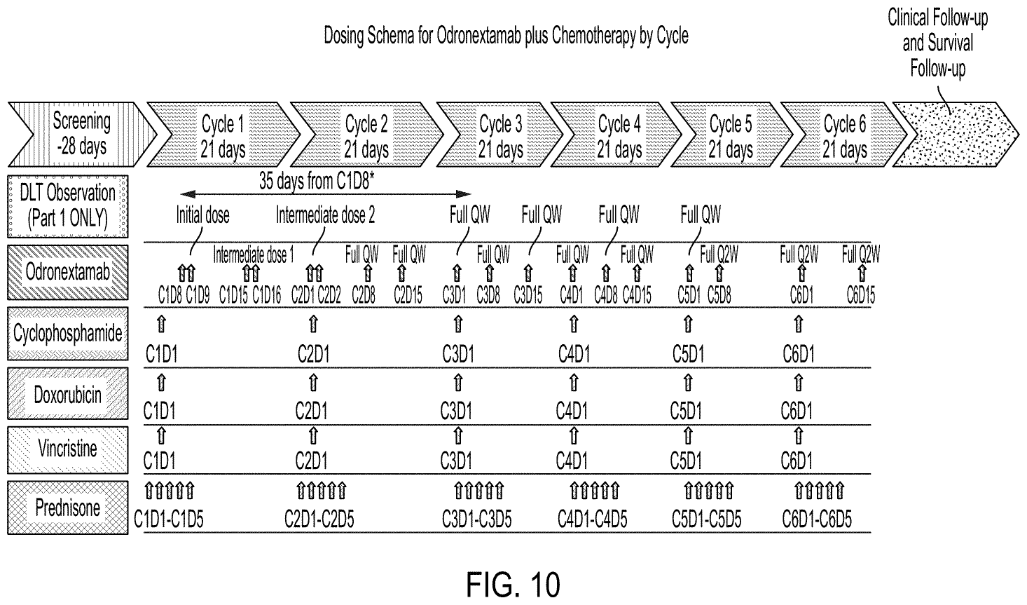

Odronextamab-chemotherapy combination will be administered for 6 cycles (1 cycle=21 days), if participant discontinues one component of the combination treatment, the other component may be administered until the end of the 6 cycles of induction period. During the treatment period, on the days that chemotherapy and odronextamab are administered on the same day, chemotherapy should be administered and completed prior to start of odronextamab infusion. Details on doses and administration of CHOP are described in Table 31, below. The dosing schema for odronextamab plus CHOP by cycle is illustrated in FIG. 10.

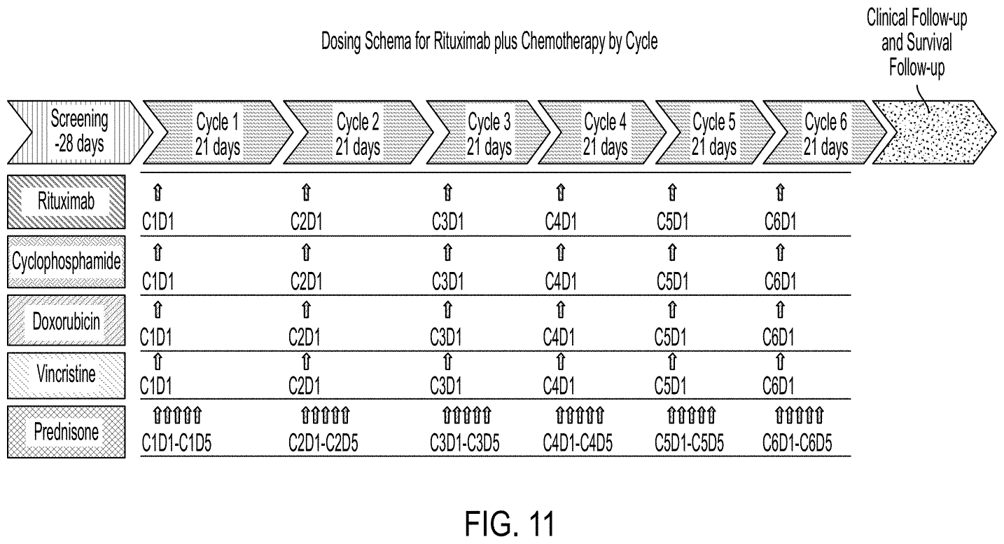

For this study, use of rituximab biosimilars is permitted. Rituximab must be administered according to institutional guidelines or according to the instructions in the product package insert. Rituximab will be administered intravenously on day 1 of each cycle at a dose of 375 mg/m². The frequency and duration of rituximab is every 3 weeks (Q3W) in combination with CHOP for 6 cycles of 21 days length (treatment period). The dosing schema for rituximab plus CHOP by cycle is illustrated in FIG. 11.

CHOP must be administered according to standard of care (SoC), institutional guidelines or according to the instructions in the product package inserts. For details about CHOP, including formulation and administration, please refer to the product package insert.

TABLE 31
CHOP Dose

| | | | | | | Cycle Length |
|---|---|---|---|---|---|---|
| | Drug | Dose | | Route | Days | (Days)/# of cycles |
| CHOP | Cyclophosphamide | 750 | mg/m² | IV | 1 | 21 days/ |
| | | | | | | 6 cycles |
| | Doxorubicin | 50 | mg/m² | IV | 1 | 21 days/ |
| | Vincristine | 1.4 mg/m² | | | | 6 cycles |
| | | (max 2 mg) | | | | |
| | Prednisone | 100 | mg | PO | 1-5 | 6 cycles |

PO = orally;
IV = intravenous

Premedications to mitigate the risk and reduce the severity of CRS are detailed in Example 3. CRS toxicity grading is an shown in, e.g., Table 6.

### Example 16: Clinical Evaluation of Odronextamab (REGN1979), an Anti-CD20 x Anti-CD3 Bispecific Antibody, in Combination with Chemotherapy Versus Rituximab in Combination with Chemotherapy in Previously Untreated Participants with Follicular Lymphoma

This is a phase 3, open-label, randomized, multicenter study of odronextamab in combination with chemotherapy compared to rituximab in combination with chemotherapy for previously untreated participants with follicular lymphoma. The study consists of 2 Parts: Part 1 of the study is a safety run-in to assess the safety and tolerability of and determine the intended dose regimen of odronextamab in combination with chemotherapy for Part 2; Part 2 is the randomized part of the study, evaluating the efficacy and safety of odronextamab in combination with chemotherapy in comparison to rituximab in combination with chemotherapy.

In Part 1 (Safety Run-In) the safety of odronextamab in combination with chemotherapy (cyclophosphamide, doxorubicin, vincristine, and prednisone, collectively [CHOP] or cyclophosphamide, vincristine, and prednisone, collectively [CVP]) will be evaluated as the primary objective, and in part 2 the anti-tumor activity of odronextamab in combination with chemotherapy (CHOP or CVP) versus rituximab in combination with chemotherapy (CHOP or CVP) will be assessed as primary objective. As disclosed herein, chemotherapy with CHOP or CVP will be referred to simply as ‘chemotherapy’. Up to 48 participants may be enrolled. Odronextamab will start at a lower dose (Dose Level 1 of 40 mg) with a planned dose escalation, if tolerated, to Dose Level 2 of 80 mg. Dose-limiting toxicities (DLTs) and the tolerability of odronextamab-CHOP and odronextamab-CVP will be assessed. All participants will receive odronextamab with investigator-selected chemotherapy regimen (CHOP or CVP) in Part 1. Participants will receive 6 cycles of odronextamab in combination with chemotherapy. The first dose of odronextamab will be administered a week after initiating chemotherapy (on C1D8) to mitigate the risk for CRS. At the end of cycle 6 (W24 assessment), participants with CR or PR will continue to the maintenance therapy period with monotherapy treatment with odronextamab. Participants who relapse during treatment or are in stable disease (SD) after induction will continue to the safety follow up and efficacy follow-up. Table 32 provides the dosing schema of odronextamab for combination (with CHOP or CVP) in Part 1 (Safety Run-in). Dosing level will commence with 80 mg odronextamab full dose (DL 1). CHOP/CVP doses remain the same across dose levels; CHOP/CVP begins on cycle 1 day 1 (C1D1) for all DLs. CHOP/CVP is given every 21 days for a total of 6 cycles (1 cycle=21 days). Odronextamab will commence on C1D8 for all DLs.

TABLE 32
Odronextamab Dose Escalation Schema

| | | | DL 1 | DL 2 |
|---|---|---|---|---|
| | | | Odronextamab | Odronextamab |
| Cycle | Day | Dosing Schema | Dose (mg) | Dose (mg) |
| C1 | D 1 | N/A | N/a | N/A |
| | D 8 | Initial Dose | 0.2 | 0.2 |
| | D 9 | | 0.5 | 0.5 |
| | D 15 | Intermediate Dose 1 | 2 | 2 |
| | D 16 | | 2 | 2 |
| C2 | D 1 | Intermediate Dose 2 | 10 | 10 |
| | D 2 | | 10 | 10 |
| | D 8 | QW | 40 | 80 |
| | D 15 | | 40 | 80 |
| C3 | D 1 | | 40 | 80 |
| | D 8 | QW | 40 | 80 |
| | D 15 | | 40 | 80 |
| C4 | D 1 | | 40 | 80 |
| | D 8 | | 40 | 80 |
| | D 15 | | 40 | 80 |
| C5 | D 1 | | 40 | 80 |
| C6 | D 8 | Q2W | 80 | 160 |
| | D 1 | | 80 | 160 |
| | D 15 | | 80 | 160 |

C = cycle;
D = day;
QW = weekly;
Q2W = every 2 weeks
Note:
If DL1 is intolerable, data will be evaluated to consider lower doses. Doses of chemotherapy agents do not change across dose levels. During cycles C7 to 18, 320 mg may be administered every eight weeks.

Approximately 669 participants will be enrolled and will be randomly assigned in a 1:1:1 ratio to receive (A) odronextamab in combination with chemotherapy with no maintenance, (B) odronextamab in combination with chemotherapy followed by odronextamab maintenance, or (C) rituximab in combination with chemotherapy followed by rituximab maintenance. Selection of chemotherapy (CHOP or CVP) will be according to the investigator's clinical judgement and will be made prior to randomization. Randomization will be stratified according to Follicular Lymphoma International Prognostic Index 1 (FLIPI 1) score (0 or 1 [low risk], 2 [intermediate risk], or 3 to 5 [high risk]), choice of chemotherapy, longest lesion diameter (6 vs. >6 cm), and age (<65 vs. ≥65 years old). For participants assigned to receive odronextamab in combination with chemotherapy, odronextamab will be given in combination with 6 cycles of induction chemotherapy. In Arm B, treatment with odronextamab monotherapy will continue for participants with CR and PR with a dose of 320 mg every 8 weeks (Q8W) for up to 12 doses or until disease progression, loss to follow up or withdrawal of consent, whichever is earlier. For participants assigned to receive rituximab combined with chemotherapy (Arm C), the treatment will be per standard practice, 6 cycles of induction chemotherapy, followed by up to 12 doses of rituximab monotherapy at Q8W intervals (participants with CR and PR only) or until disease progression, loss to follow up or withdrawal of consent whichever is earlier.

The study population will consist of participants of 18 years and older with previously untreated CD20⁺ FL based on World Health Organization (WHO) classification.

Inclusion Criteria: A participant must meet the following criteria to be eligible for inclusion in the study:
1. Have diagnoses of CD20⁺ FL Grade 1-3a, stage II bulky or stage III/IV: Local histopathologic confirmation of the CD20⁺ FL Grade 1 to 3a, must be obtained before study enrollment. Biopsies must have been obtained within 18 months prior to study enrollment. A corresponding tumor biopsy sample should be submitted to the central laboratory. For Part 1 (Safety Run-In) only: FLIP1 score of 3 to 5.
2. Need for treatment as indicated: Presence of ≥1 of the following: B symptoms, large tumor mass (characterized by lymphomas with a diameter >3 cm in 3 or more regions or by a lymphoma with a diameter >6 cm in 1 region), and/or presence of lymphoma-related complications.
3. Have measurable disease on cross-sectional imaging documented by diagnostic imaging CT or MRI (measurable disease is defined as at least 1 bidimensionally measurable nodal lesion of >1.5 cm or extranodal disease of >1 cm in the greatest transverse diameter (GTD) regardless of the short axis diameter).
4. Eastern Cooperative Oncology Group (ECOG) performance status of 0-2.
5. Age ³ 18 years
6. Adequate bone marrow function as documented by:
   a. Platelet count ³ 50×10⁹/L. A participant may not have received platelet transfusion therapy within 7 days prior to first dose of odronextamab in order to meet the platelet eligibility criterion.
   b. Hemoglobin ³ 9.0 g/dL
   c. Absolute neutrophil count (ANC) ³ 1.0×10⁹/L. A participant may not have received G-CSF within 2 days prior to the first dose of odronextamab in order to meet the ANC eligibility criterion.
   d. Participants with bone marrow involvement or splenic sequestration should meet the following hematologic parameters:
      Platelet count ³ 25×10⁹/L. A participant may not have received platelet transfusion therapy within 3 days prior to first dose of odronextamab in order to meet the platelet eligibility criterion.
      Hemoglobin ³ 7.0 g/dL
      ANC≥0.5×10⁹/L. A participant may not have received G-CSF within 2 days prior to first dose of odronextamab in order to meet the ANC eligibility criterion.
7 Participants with adequate hepatic function:
   a. Total bilirubin ≤1.5×upper limit of normal (ULN) (≤3×ULN if attributed to lymphoma infiltration of liver).
   b. Alanine aminotransferase (ALT) and aspartate aminotransferase (AST)≤2.5×ULN (≤5×ULN if attributed to lymphoma infiltration of liver).
   c. Alkaline phosphatase (ALP)≤2.5×ULN (≤5×ULN if attributed to lymphoma infiltration of liver).
   NOTES:
      Irrespective of the presence of lymphoma infiltration of the liver, a participant with an AST>2.5×ULN and/or ALT>2.5×ULN concurrent with a total bilirubin >1.5×ULN will be excluded.
      Participants with known Gilbert syndrome will be excluded if the total bilirubin value is >4×ULN for the local general population.
8. Calculated creatinine clearance by Cockcroft-Gault formula ³ 50 mL/min.
   NOTE: Participants with a calculated creatinine clearance <50 mL/min may be considered for enrollment if a measured creatinine clearance (based on 24-hour urine collection or other reliable method) is ≥50 mL/min.
9. Ability to understand the purpose and risks of the study and provide signed and dated informed consent and authorization to use protected health information by study participant or legally acceptable representative (in accordance with national and local participant privacy regulations).
10. Willing and able to comply with clinic visits and study-related procedures.
11. Able to understand and complete study-related questionnaires.

Exclusion Criteria: A participant who meets any of the following criteria will be excluded from the study:
1. Participants with central nervous system (CNS) lymphoma or leptomeningeal lymphoma.
   a. Primary CNS lymphoma or known involvement by non-primary CNS lymphoma (even if treated into complete remission).
   b. Suspected CNS involvement by lymphoma must be evaluated by CNS imaging (MRI or CT) and by lumbar puncture as appropriate.
2. Participants with histological evidence of transformation to a high-grade or diffuse large B-cell lymphoma.
3. Participants with Waldenström macroglobulinemia (WM, lymphoplasmacytic lymphoma), Grade 3b follicular lymphoma, chronic lymphocytic leukemia, or small lymphocytic lymphoma.
4. Treatment with any systemic anti-lymphoma therapy.
5. Recent major surgery (within 4 weeks prior to the start of assigned study treatment).
6. Standard radiotherapy within 14 days of first administration of assigned study treatment.
7. History of solid organ transplantation.
8. Continuous systemic corticosteroid treatment with more than 10 mg per day of prednisone or anti-inflammatory equivalent within 72 hours of start of study drug.
9. A malignancy other than NHL unless the participant is adequately and definitively treated and is cancer free for at least 3 years with the exception of localized prostate cancer treated with hormone therapy or local radiotherapy (i.e. pellets), cervical carcinoma in situ, breast cancer in situ, or nonmelanoma skin cancer that was definitively treated.
10. Any other significant active disease or medical condition that could interfere with the conduct of the study or put the participant at significant risk, including but not limited to significant cardiovascular disease (eg, New York Heart Association Class III or IV cardiac disease, myocardial infarction within the previous 6 months, unstable arrhythmias, or unstable angina); significant pulmonary disease (eg, obstructive pulmonary disease and history of symptomatic bronchospasm); or gastrointestinal, hepatic, renal, endocrine, hematologic, autoimmune, psychiatric, or neurologic disorder.
11. History of or current relevant CNS pathology, such as epilepsy, seizure, paresis, aphasia, apoplexy, severe brain injury, cerebellar disease, organic brain syndrome, psychosis, inflammatory lesions, and/or vasculitis.
12. Vaccination within 28 days prior to first study drug administration with a vector that has replicative potential.
13. Cardiac ejection fraction <50% by echocardiogram or multigated acquisition (MUGA) scan.
14. Pregnant or breastfeeding women.
15. Women of childbearing potential (WOCBP)* or men who are unwilling to practice highly effective contraception prior to the initial dose/start of the first treatment, during the study, and for at least 6 months after the last dose. Sperm donation is prohibited during the study and for 6 months after the last dose of study drug. Highly effective contraceptive measures include:
   a. stable use of combined (estrogen and progestogen containing) hormonal contraception (oral, intravaginal, transdermal) or progestogen-only hormonal contraception (oral, injectable, implantable) associated with inhibition of ovulation initiated 2 or more menstrual cycles prior to screening;
   b. intrauterine device (IUD); intrauterine hormone-releasing system (IUS);
   c. bilateral tubal occlusion/ligation;
   d. vasectomized partner (provided that the male vasectomized partner is the sole sexual partner of the WOCBP study participant and that the vasectomized partner has obtained medical assessment of surgical success for the procedure); and/or
   e. sexual abstinence.
16. History of clinically significant cardiovascular, respiratory, hepatic, renal, gastrointestinal, endocrine, hematological, psychiatric, or neurological disease, as assessed by the investigator, that may confound the results of the study or poses an additional risk to the participant by study participation.
17. Has a history of tuberculosis or systemic fungal diseases that has been active within 6 months.
18. Infections:
   Infection requiring hospitalization or treatment with IV anti-infectives within 2 weeks of first administration of assigned study treatment. There should be evidence that the infection has cleared or is well controlled by start of study therapy.
   Evidence of any active infection (bacterial, viral, fungal, mycobacterial, parasitic, or other) at study enrollment or within 2 weeks of study enrollment, if requiring ongoing treatment and/or has the potential to cause disseminated disease or severe infection upon immunosuppression.
   Active COVID-19 infection defined as PCR+
   Uncontrolled infection with human immunodeficiency virus (HIV), hepatitis B (HBV), or hepatitis C (HCV).
   Cytomegalovirus (CMV) infection as noted by detectable levels on peripheral blood PCR assay. Participants who show detectable levels of CMV at screening will need to be treated with appropriate antiviral therapy and demonstrate at least 2 undetectable levels of CMV by PCR assay (at least 7 days apart) before being re-considered for eligibility.
   NOTE: Participants with HIV who have controlled infection (undetectable viral load and CD4 count above 350 cells/μL either spontaneously or on a stable antiviral regimen) are permitted.
   NOTE: Participants who are hepatitis B surface antigen positive or who are hepatitis B core antibody positive should undergo evaluation by a specialist and be considered for antiviral prophylaxis, before they are permitted onto study.
   NOTE: Participants who are hepatitis C virus antibody positive (HCV Ab+) who have controlled infection (undetectable HCV RNA by PCR either spontaneously or in response to a successful prior course of anti-HCV therapy) are permitted.
19. Allergy/hypersensitivity:
   a. History of severe allergic reaction attributed to compounds with a similar chemical or biologic composition as that of the study drug or excipient.
   b. Known hypersensitivity to both allopurinol and rasburicase.
20. Participated in any clinical research study evaluating another investigational drug including biologics or therapy, including specific immunotherapy, within 90 days or at least 5 half-lives (whichever is longer) of an investigational biologic drug, or at least 4 weeks for other investigational drug, prior to the screening visit.
21. Participants who are committed to an institution by virtue of an order issued either by the judicial or the administrative authorities.

The study interventions administered in this study are shown in Table 33, below.

TABLE 33
Study Interventions Administered

| | Arm 1: | Arm 2: | | | | |
|---|---|---|---|---|---|---|
| | O-chemotherapy | R-chemotherapy | Chemotherapy | | | |
| | Intervention Name | | | | | |
| | Odronextamab | Rituximab | CP | DX | VC | PN |
| Dosage Level (s) | See Table 34 | 375 mg/m² | 750 mg/m² | 50 mg/m² | 1.4 mg/m² | 100 mg |
| Route of | IV infusion | IV infusion | IV | IV | IV | PO |
| Administration | | | | | | |

CP—cyclophosphamide;
DX—doxorubicin;
VC—vincristine;
PN—prednisone

Odronextamab will be administered in combination with chemotherapy for 6 cycles of 21 days. CHOP or CVP will be dosed on C1D1 and odronextamab dosing will begin on C1D8 to decrease the risk for CRS. During the induction treatment period, odronextamab is administered intravenously with step-up dosing in cycles 1 and 2 to mitigate the risk of CRS. Step-up dosing consists of an initial dose of 0.7 mg (split as 0.2 mg on C1D8 and 0.5 mg on C1D9), an intermediate dose 1 of 4 mg (split as 2 mg on C1D15 and 2 mg on C1D16), followed by intermediate dose 2 of 20 mg (split as 10 mg on C2D1 and 10 mg on C2D2). From C2D8 up to C5D1, odronextamab is administered intravenously weekly on days 1 (except cycle 2), 8 and 15 at 40 or 80 mg. From C5D8, Q2W administration of odronextamab will commence with doses on C5D8 and C6D1 and C6D15. See Table 34. From cycle 7 to 18, Q8W maintenance therapy includes 320 mg odronextamab.

TABLE 34
Odronextamab Administration
Odronextamab Dose Regimen

| | | | | | | Weekly dose (QW) | Every 2 |
|---|---|---|---|---|---|---|---|
| | | | | | | Cycle 2: Day 8, | Weeks dose |
| | | Intermediate 1 | | Intermediate 2 | | Day 15 | (Q2W) |
| Initial (0.7 mg) | | (4 mg) | | (20 mg) | | Cycle 3 to 4: | Cycle 5: Day 8 |
| Cycle 1 | Cycle 1 | Cycle 1 | Cycle 1 | Cycle 2 | Cycle 2 | Day 1, Day 8 and 15 | Cycle 6: Days |
| Day 8 | Day 9 | Day 15 | Day 16 | Day 1 | Day 2 | Cycle 5: Day 1 | 1 and 15 |
| 0.2 mg | 0.5 mg | 2 mg | 2 mg | 10 mg | 10 mg | 40 or 80 mg | 80 or 160 mg |

For the initial dose, intermediate dose 1 and intermediate dose 2, the treatment will be split into 2 separate infusions given on 2 separate days which are preferably consecutive, but no more than 3 days apart. Each of the split infusion during cycle 1 and cycle 2 and the first full dose QW infusion (C2D8) should occur over 4 hours. Subsequent treatments may be administered as a single infusion or as 2 separate infusions over at least 1 hour depending on tolerability. Administration of the C2D8 full dose will only proceed if the initial (0.7 mg) and intermediate dose 1 (4 mg) and intermediate dose 2 (20 mg) were received and tolerated (participants who received >90% of the intended dose are considered to have received the full dose).

Odronextamab-chemotherapy combination will be administered for 6 cycles (1 cycle=21 days), if participant discontinues one component of the combination treatment, the other component may be administered until the end of the 6 cycles of induction period. During the treatment period, on the days that chemotherapy and odronextamab are administered on the same day, chemotherapy should be administered and completed prior to start of odronextamab infusion. Details on doses and administration of CHOP/CVP are described in Table 35, below. The dosing schema for odronextamab plus CHOP/CVP by cycle is illustrated in FIG. 10.

Odronextamab dosing will start 1 week after chemotherapy. Upon completion of the combination treatment period (or early termination of chemotherapy), participants in Part 1 or in Part 2 Arm B will continue to receive odronextamab as monotherapy for up to 12 maintenance doses (only participant who have CR or PR).

For this study, use of rituximab biosimilars is permitted. Rituximab must be administered according to institutional guidelines or according to the instructions in the product package insert. Rituximab will be administered intravenously on day 1 of each cycle at a dose of 375 mg/m². The frequency and duration of rituximab is every 3 weeks (Q3W) in combination with chemotherapy for 6 cycles of 21 days length (treatment period). The dosing schema for rituximab plus chemotherapy by cycle is illustrated in FIG. 11.

Chemotherapy must be administered according to standard of care (SoC), institutional guidelines or according to the instructions in the product package inserts. For details about CHOP/CVP, including formulation and administration, please refer to the product package insert.

TABLE 35
Chemotherapy Dose

| | | | | | | Cycle Length |
|---|---|---|---|---|---|---|
| SOC Arm | Drug | Dose | | Route | Days | (Days)/# of cycles |
| CHOP | Cyclophosphamide | 750 | mg/m² | IV | 1 | 21 days/ |
| | | | | | | 6 cycles |
| | Doxorubicin | 50 | mg/m² | IV | 1 | 21 days/ |
| | Vincristine | 1.4 mg/m² | | IV | 1 | 6 cycles |
| | | (max 2 mg) | | | | |
| | Prednisone | 100 | mg | PO | 1-5 | |
| CVP | Cyclophosphamide | 750 | mg/m² | IV | 1 | 21 days/ |
| | | | | | | 6 cycles |
| | Vincristine | 1.4 mg/m² | | IV | 1 | 21 days/ |
| | | (max 2 mg) | | | | 6 cycles |
| | Prednisone | 100 | mg | PO | 1-5 | |

PO = orally;
IV = intravenous

Premedications to mitigate the risk and reduce the severity of CRS are detailed in Example 3. CRS toxicity grading is as shown in, e.g., Table 6.

### Example 17: Clinical Evaluation of Odronextamab (REGN1979), an Anti-CD20 x Anti-CD3 Bispecific Antibody, Versus Investigator's Choice in Previously Untreated Participants with Diffuse Large B-Cell Lymphoma

This is an open-label, multicenter, randomized, phase 3 study to compare the efficacy and safety of odronextamab to investigator's choice of chemotherapy (Rituximab-CHOP, Rituximab-CVP, or Rituximab-Bendamustine) for treatment of participants with previously untreated DLBCL. In Part 1 (safety run-in), the intended dose of odronextamab monotherapy to carry forward to Part 2 (randomized phase) will be tested. The efficacy and safety of the recommended dose of odronextamab will be evaluated in Part 2 versus investigator's choice of chemotherapy. All participants will receive 6 cycles of induction. At the end of cycle 6 induction, participants with complete response (CR) or partial response (PR) will continue to the maintenance therapy period with monotherapy treatment with either odronextamab or rituximab, depending on the study arm. Participants who relapse/progress during treatment or are in stable disease (SD) at the end of induction will be withdrawn from the treatment and continue to safety and survival follow-ups.

From 12-24 patients will be enrolled in the safety lead-in part. Approximately 446 participants will be enrolled and will be randomly assigned in a 1:1 ratio to receive either (A) odronextamab for induction, followed by odronextamab maintenance, or (B) investigator's choice of immuno-chemotherapy, followed by rituximab maintenance. Selection of therapy (CHOP, CVP, or bendamustine) will be according to the investigator's clinical judgement. For participants assigned to receive odronextamab, odronextamab will be given as monotherapy for 6 cycles at the dose selected during Part 1 (see Table 36). At the end of cycle 6, based on week 24 scan assessments, participants with CR or PR will continue treatment with odronextamab monotherapy at a dose of 320 mg Q8W for up to 12 doses or until disease progression, loss to follow-up, or withdrawal of consent, whichever is earlier. For participants assigned to receive rituximab combined with chemotherapy, the treatment will be per standard practice, 6 cycles of induction chemotherapy, followed by up to 12 doses of rituximab monotherapy at Q8W intervals (participants with CR and PR only), or until disease progression, loss to follow-up, or withdrawal of consent, whichever is earlier.

TABLE 36
Odronextamab Dose De-Escalation Schema

| | Odronextamab Dose | | | | | |
|---|---|---|---|---|---|---|
| | | | | | | Q8W |
| Dose | | Intermediate | Intermediate | | | Maintenance |
| Level | Initial dose | dose 1 | dose 2 | Full dose | 2× full dose | dose |
| (DL) | Cycle 1 | | | Cycle 2 to 4 | Cycle 5 & 6 | Cycle 7 to 18 |
| 2 | QW 0.7 mg | QW 4 mg | QW 20 mg | 160 mg | 320 mg | 320 mg |
| | (split 0.2/0.5) | (split 2/2) | (split 10/10) | | | |
| 1 | QW 0.7 mg | QW 4 mg | QW 20 mg | 80 mg | 160 mg | 320 mg |
| | (split 0.2/0.5) | (split 2/2) | (split 10/10) | | | |
| −1 | QW 0.7 mg | QW 4 mg | QW 20 mg | 40 mg | 80 mg | 320 mg |
| | (split 0.2/0.5) | (split 2/2) | (split 10/10) | | | |

The study population will consist of participants 18 years and older with intermediate to high risk previously untreated diffuse large B-cell lymphoma.

Inclusion Criteria: A participant must meet the following criteria to be eligible for inclusion in the study:
1. Previously untreated participants for lymphoma with *documented CD20+ DLBCL including one of the following diagnoses as per 2016 WHO classification of lymphoid neoplasms:
   a. **DLBCL, not otherwise specified (NOS) including germinal center B-cell type, activated B-cell type.
   b. High grade B-cell neoplasm, with MYC, bcl-2 and/or bcl-6 rearrangements For Part 1 only: participants will need to have at least one of the high-risk features to be eligible. High risk features include, but are not limited to, IPI score 3 to 5, cell of origin (non-GCB type), double-hit or triple-hit lymphoma (classified as high-grade B cell neoplasm, with MYC, bcl-2 and/or bcl-6 rearrangements according to 2016 WHO classifications of lymphoid neoplasms), TP53 mutations, CDKN2A loss, etc (Sehn and Salles, 2021). *Local histopathologic confirmation of the DLBCL must be obtained before study enrollment. Biopsies must have been obtained within 3 months prior to study enrollment. Availability of tumor tissue for submission to central laboratory is required for study enrollment.**De novo or histologically transformed from a diagnosis of follicular lymphoma
2. Have measurable disease with at least one nodal lesion with LDi greater than 1.5 cm or at least one extranodal lesion with LDi greater than 1.0 cm, documented by diagnostic imaging (computed tomography [CT] or magnetic resonance imaging [MRI]).
3. Age ≥18 years
4. Eastern Cooperative Oncology Group (ECOG) performance status
5. Life expectancy ≥12 months
6. International Prognostic Index (IPI)≥2
7. Adequate hematologic function, as measured by:
   a. Platelet count ≥75×109/L. A participant may not have received platelet transfusion within 7 days of first dose of the assigned study treatment in order to meet this eligibility requirements
   b. Absolute neutrophil count (ANC)≥1.0×109/L. A participant may not have received granulocyte colony stimulating factor (G-CSF) within 2 days of first dose of the assigned study treatment in order to meet this eligibility requirement
   c. Hemoglobin level ≥9 g/dL
NOTE: Participants with cell counts below thresholds listed above may be considered for enrollment if, in the opinion of the investigator, the reason is believed to be due to bone marrow infiltration or splenic sequestration by the underlying disease.
   Participants with bone marrow involvement or splenic sequestration should meet the following hematologic parameters:
      a. Platelet count ≥25×109/L. A participant may not have received platelet transfusion therapy within 3 days prior to first dose of study treatment in order to meet the platelet eligibility criterion.
      b. Absolute neutrophil count (ANC)≥0.5×109/L. A participant may not have received granulocyte colony stimulating factor within 2 days prior to first dose of study treatment in order to meet the ANC eligibility criterion.
      c. Hemoglobin ≥9.0 g/dL
8. Adequate organ function, as documented by:
   a. Cardiac ejection fraction >50% by echocardiogram or multigated acquisition (MUGA) scan
   b. Total bilirubin ≤1.5×upper limit of normal (ULN) (≤3×ULN if attributed to lymphoma infiltration of liver)
   c. Alanine aminotransferase (ALT) and aspartate aminotransferase (AST)≤2.5×ULN (≤5×ULN if attributed to lymphoma infiltration of liver)
   d. Alkaline phosphatase (ALP)≤2.5×ULN (≤5×ULN if attributed to lymphoma infiltration of liver)
   e. Serum creatinine ≤1.5×ULN, or calculated creatinine clearance by Cockcroft Gault formula ≥50 mL/min
   NOTE: Irrespective of the presence of lymphoma infiltration of the liver, a participant with an AST>3×ULN and/or ALT>3×ULN concurrent with a total bilirubin >1.5×ULN will be excluded.
   NOTE: Participants with known Gilbert syndrome will be excluded if the total bilirubin value is >4×ULN.
   NOTE: Participants with a calculated creatinine clearance <50 mL/min may be considered for enrollment if a measured creatinine clearance (based on 24-hour urine collection or other reliable method) is ≥50 mL/min.
9. Willing and able to comply with clinic visits and study-related procedures.
10. Provide informed consent signed by study participant or legally acceptable representative.
11. Able to understand and complete study-related questionnaires with physical assistance, if required.

Exclusion Criteria: A participant who meets any of the following criteria will be excluded from the study:
1. Primary central nervous system (CNS) lymphoma or known involvement by non-primary CNS NHL
2. History of or current relevant CNS pathology, such as:
   a. epilepsy, seizure, paresis, aphasia, apoplexy, severe brain injury, cerebellar disease, organic brain syndrome, psychosis, or
   b. evidence for presence of inflammatory lesions and/or vasculitis on cerebral MRI
3. Peripheral neuropathy Grade
4. Another active malignancy (aside from B-cell NHL) in the past 5 years, with the following exceptions: non-melanoma skin cancer that has undergone potentially curative therapy, in situ cervical carcinoma, or any other tumor that has been deemed to be effectively treated with definitive local control and with curative intent
5. Any other significant active disease or medical condition that could interfere with the conduct of the study or put the participant at significant risk, including but not limited to significant cardiovascular disease (eg, New York Heart Association Class III or IV cardiac disease, myocardial infarction within the previous 6 months, unstable arrhythmias, or unstable angina) or significant pulmonary disease (eg, obstructive pulmonary disease and history of symptomatic bronchospasm)
6. Treatment with any systemic anti-lymphoma therapy
7 Any investigational therapy within 28 days or 5 half-lives of the drug, whichever is shorter, prior to the start of study treatment
8. Recent major surgery (within 4 weeks prior to the start of study treatment)
9. Standard radiotherapy within 14 days of first administration of study treatment
10. Prior organ transplantation
11. Allergy/hypersensitivity:
   a. Known hypersensitivity to both allopurinol and rasburicase
   b. History of allergic reactions or hypersensitivity attributed to compounds of similar chemical or biological components included in CHOP or rituximab
12. Infections:
   a. Evidence of any active infection (bacterial, viral, fungal, mycobacterial, parasitic or other) at study enrollment or within 2 weeks of study enrollment, if requiring ongoing treatment and/or has the potential to cause disseminated disease or severe infection upon immunosuppression. There should be evidence that the infection has cleared or is well controlled by start of study therapy.
   b. Active COVID-19 infection
   c. Uncontrolled infection with human immunodeficiency virus (HIV), hepatitis B virus (HBV), or hepatitis C virus (HCV)
   d. Cytomegalovirus (CMV) infection as noted by detectable levels on peripheral blood polymerase chain reaction (PCR) assay. Patients who show detectable levels of CMV at screening will need to be treated with appropriate antiviral therapy and demonstrate at least 2 undetectable levels of CMV by PCR assay (at least 7 days apart) before being re-considered for eligibility.
   NOTE: Patients with HIV who have controlled infection (undetectable viral load and CD4 count above 350 cells/μL either spontaneously or on a stable antiviral regimen) are permitted
   NOTE: Patients who are hepatitis B surface antigen positive or who are hepatitis B core antibody positive should undergo evaluation by a specialist and be considered to have controlled infection (serum hepatitis B virus DNA PCR that is below the limit of detection AND receiving anti-viral therapy for hepatitis B) before they are permitted onto study
   NOTE: Patients who are HCV antibody positive who have controlled infection (undetectable HCV RNA by PCR either spontaneously or in response to a successful prior course of anti-HCV therapy) are permitted
13. Administration of live vaccination within 28 days of study first dose (COVID-19 vaccine can be administered no less than one week prior to first dose)
14. Participants who are committed to an institution by an order issued either by the judicial or the administrative authorities (for sites in Germany only)
15. Members of the clinical site study team and/or his/her immediate family, unless prior approval granted by the Sponsor.
16. Pregnant or breastfeeding women.
17. Women of childbearing potential (WOCBP)* or men who are unwilling to practice highly effective contraception prior to the initial dose/start of the first treatment, during the study, and for at least 6 months after the last dose. Sperm donation is prohibited during the study and for 6 months after the last dose of the assigned study treatment. Highly effective contraceptive measures include:
   a. stable use of combined (estrogen and progestogen containing) hormonal contraception (oral, intravaginal, transdermal) or progestogen-only hormonal contraception (oral, injectable, implantable) associated with inhibition of ovulation initiated 2 or more menstrual cycles prior to screening
   b. intrauterine device (IUD); intrauterine hormone-releasing system (IUS)
   c. bilateral tubal ligation/occlusion
   d. vasectomized partner (provided that the male vasectomized partner is the sole sexual partner of the WOCBP study participant and that the vasectomized partner has obtained medical assessment of surgical success for the procedure)
   e. sexual abstinence

The study interventions administered in this study are shown in Table 37, below.

TABLE 37
Study Interventions Administered

| | Arm 1 | Arm 2 | | | | | |
|---|---|---|---|---|---|---|---|
| | Intervention Name | | | | | | |
| | Odronextamab | Rituximab | CP | DX | VC | PN | BD |
| Dosage Level(s) | See Table 38 | 375 mg/m² | 750 mg/m² | 50 mg/m² | 1.4 mg/m² | 100 mg | 90 mg/m² |
| Route of | IV infusion | IV infusion | IV | IV | IV | PO | IV |
| Administration | | | | | | | |

CP—cyclophosphamide;
DX—doxorubicin;
VC—vincristine;
PN—prednisone;
BD—bendamustine

Odronextamab dose administered is a flat dose and not dependent on participant weight or body surface area. During the induction treatment period, odronextamab will be administered IV with step-up dosing in cycle 1 to mitigate the risk for CRS. Cycle 1 will consist of an initial dose of 0.7 mg (split as 0.2 mg on cycle 1 day 1 and 0.5 mg on cycle 1 day 2), an intermediate dose 1 of 4 mg (split as 2 mg on cycle 1 day 8 and 2 mg on cycle 1 day 9), and an intermediate dose 2 of 20 mg (split as 10 mg on cycle 1 day 15 and 10 mg on cycle 1 day 16). From cycle 2 to cycle 4, odronextamab will be administered IV on days 1, 8, and 15 at 160 mg, and from cycle 5 and cycle 6, odronextamab will be administered IV at 320 mg on day 8 in cycle 5 and days 1 and 15 in cycle 6 (Table 38). During the monotherapy maintenance treatment period, odronextamab will be administered IV Q8W at 320 mg (Table 38). The first dose of odronextamab (320 mg) during maintenance will be administered 6 weeks after the last dose (320 mg) given during induction on cycle 6 day 15.

TABLE 38
Odronextamab Administration
Odronextamab Dose Regimen

| | | | | | | | | Cycle 6 | |
|---|---|---|---|---|---|---|---|---|---|
| Cycle 1 | Cycle 1 | Cycle 1 | Cycle 1 | Cycle 1 | Cycle 1 | Cycle 2-4 | Cycle 5 | Day 1 | M1 to M12 |
| Day 1 | Day 2 | Day 8 | Day 9 | Day 15 | Day 16 | Day 1, 8 | Day 8 | and 15 | Day 1 |
| Initial | | Intermediate 1 | | Intermediate 2 | | and 15 | 2× full | 2× full | Maintenance |
| (0.7 mg) | | (4 mg) | | (20 mg) | | Full dose | dose | dose | Q8W |
| 0.2 mg | 0.5 mg | 2 mg | 2 mg | 10 mg | 10 mg | 160 mg | 320 mg | 320 mg | 320 mg |

For the initial dose, intermediate dose 1, and intermediate dose 2, the treatment will be split into 2 separate infusions given on 2 separate days, which are preferably consecutive but no more than 3 days apart. Each of the split infusions during cycle 1 and the first full dose QW infusion (cycle 2 day 1) should occur over 4 hours. Subsequent treatments may be administered as a single infusion or as 2 separate infusions and may be administered over at least 1 hour depending on tolerability.

Premedications to mitigate the risk and reduce the severity of CRS are detailed in Example 3. CRS toxicity grading is an shown in, e.g., Table 6.

For this study, use of rituximab or biosimilars is permitted. Rituximab must be administered according to institutional guidelines or according to the instructions in the product package insert. Rituximab will be administered IV on day 1 of each cycle in a dose of 375 mg/m². The frequency and duration of rituximab will be Q3W in combination with induction chemotherapy (CHOP/CVP) for 6 cycles of 21 days each and Q4W with induction bendamustine for 6 cycles of 28 days each (induction treatment phase). Upon completion of the combination treatment period or early termination of chemotherapy, rituximab will be continued as monotherapy Q8W for up to 12 maintenance doses (if participant has CR or PR), unless the participant discontinues early due to toxicity, progressive disease, or start of subsequent lymphoma therapy or due to discretionary reasons (participant or investigator). Rituximab and chemotherapy administration is described in Table 39. For details about CHOP/CVP and bendamustine, including formulation and administration, refer to the product package insert. Eight weeks after the dose on cycle 6 day 1, participants (who had CR or PR) will start the maintenance treatment period of rituximab monotherapy on a Q8W schedule.

TABLE 39
CHOP/CVP and Bendamustine Dose

| Investigator's Choice | | | | | | Cycle Length |
|---|---|---|---|---|---|---|
| Arm | Drug | Dose | | Route | Days | (Days)/# of cycles |
| CHOP | Cyclophosphamide | 750 | mg/m² | IV | 1 | 21 days/ |
| | | | | | | 6 cycles |
| | Doxorubicin | 50 | mg/m² | IV | 1 | 21 days/ |
| | Vincristine | 1.4 mg/m² | | IV | 1 | 6 cycles |
| | | (max 2 mg) | | | | |
| | Prednisone | 100 | mg | PO | 1-5 | |
| CVP | Cyclophosphamide | 750 | mg/m² | IV | 1 | 21 days/ |
| | | | | | | 6 cycles |
| | Vincristine | 1.4 mg/m² | | IV | 1 | 21 days/ |
| | | (max 2 mg) | | | | 6 cycles |
| | Prednisone | 100 | mg | PO | 1-5 | |
| Bendamustine | Bendamustine | 90 | mg/m² | IV | 1-2 | 28 days/ |
| | | | | | | 6 cycles |

### Example 18: Clinical Evaluation of Odronextamab Versus Standard of Care Therapy in Patients with Relapsed/Refractory Aggressive B-Cell Non-Hodgkin's Lymphoma

This is an open label, multicenter, randomized phase 3 study to compare event-free survival of odronextamab monotherapy (arm 1) versus standard of care (SOC) (arm 2; salvage therapy followed by autologous stem cell transplant [ASCT]) for participants with relapsed/refractory (R/R) aggressive B-NHL.

Participants in arm 1 will receive 4 cycles (21-days per cycle) of induction treatment administered intravenously (IV) with step-up dosing in cycle 1 (C1) to mitigate risk of cytokine release syndrome (CRS). Cycle 1 consists of an initial dose of 0.7 mg (split as 0.2 mg on cycle 1 day 1 [C1D1] and 0.5 mg on C1D2), an intermediate dose-1 of 4 mg (split as 2 mg on C1D8 and 2 mg on C1D9), followed by intermediate dose-2 of 20 mg (split as 10 mg on C1D15 and 10 mg on C1D16). From cycle 2 up to cycle 4, odronextamab is administered intravenously at the recommended phase 2 dose (RP2D) of 160 mg on cycle D1, D8, and D15.

Participants without progressive disease during induction will continue to maintenance treatment with odronextamab 320 mg every 2 weeks (Q2W) until 1 year from start of treatment or progressive disease or death due to any cause, whichever occurs first.

Participants in arm 2 (SOC) will receive up to 3 cycles of salvage therapy (ifosfamide, carboplatin, etoposide±rituximab [ICE±R], or dexamethasone, cisplatin, cytarabine±rituximab [DHAP±R], or gemcitabine, dexamethasone, cisplatin±rituximab [GDP±R]) and continue with ASCT following a complete response (CR)/partial response (PR). For participants with chemotherapy toxicity or sub-optimal response, a switch between the pre-defined salvage regimens is allowed and will not be counted as an event. Participants with no optimal response following salvage therapy or at any time during ASCT treatment period, participants may cross over to receive odronextamab treatment for 1 year per arm 1, and this will be recorded as an event in arm 2 prior to crossover.

The study population will consist of participants of 18 years and older with R/R aggressive B-NHL (based on 2016 World Health Organization [WHO] classification) and who are refractory to or have relapsed within 12 months of anti-CD20 antibody and anthracycline-containing frontline therapy.

Participants with the following criteria will be enrolled in this study:
Participants with R/R within 1-year after frontline therapy with documented aggressive B-NHL and intent to proceed with ASCT
Have measurable disease with at least one nodal lesion with LDi greater than 1.5 cm or at least one extranodal lesion with LDi greater than 1.0 cm, documented by diagnostic imaging (CT or MRI)
ECOG performance status of 0 to 1
Evidence of adequate hematologic, hepatic, renal, pulmonary, and cardiac function

During the induction treatment period, odronextamab is administered IV with step-up dosing in cycle 1 to mitigate risk of CRS. Cycle 1 consists of an initial dose of 0.7 mg (split as 0.2 mg on C1D1 and 0.5 mg on C1D2), an intermediate dose-1 of 4 mg (split as 2 mg on C1D8 and 2 mg on C1D9), followed by intermediate dose-2 of 20 mg (split as 10 mg on C1D15 and 10 mg on C1D16).

From cycle 2 up to cycle 4, odronextamab is administered intravenously on cycle D1, D8, and D15 at 160 mg. Starting at maintenance treatment 1 (and up to 18 doses), during monotherapy maintenance treatment odronextamab will be administered IV Q2W at 320 mg dose.

Salvage therapy will be administered per label for up to 3 cycles and will be selected from one of the following:
ICE±R (ifosfamide, carboplatin, etoposide±rituximab) given as follows:
rituximab 375 mg/m² on day 1, ifosfamide 5 gm/m² on day 2, etoposide 100 mg/m² on days 1 to 3, carboplatin AUC=5 (dose capped at 800 mg) on day 2
DHAP±R (dexamethasone, cisplatin, cytarabine±rituximab) given as follows:
rituximab 375 mg/m² on day 1, dexamethasone 40 mg on days 1 to 4, cisplatin 100 mg/m² on day 1 and cytarabine 2 doses of 2 gm/m² each on day 2
GDP±R (gemcitabine, dexamethasone, cisplatin±rituximab) given as follows:
rituximab 375 mg/m² on day 1, cisplatin 75 mg/m² on day 1, dexamethasone 40 mg on days 1 to 4 and gemcitabine 1 gm/m² on days 1 and 8

Leukapheresis will be performed during this time as per local institutional guidelines, unless the participant has stem cells already collected prior to study entry. ASCT will be administered per SOC and will include standard conditioning regimen and a post-ASCT recovery period

Premedications to mitigate the risk and reduce the severity of CRS are detailed in Example 3. CRS toxicity grading is as shown in, e.g., Table 6.

### Example 19: Clinical Evaluation of Odronextamab (REGN1979), an Anti-CD20 x Anti-CD3 Bispecific Antibody, for the Treatment of Relapsed/Refractory Marginal Zone Lymphoma (R/R MZL)

Marginal zone lymphoma (MZL) is a heterogeneous disease comprising 3 subtypes, extra-nodal MZL of mucosa-associated lymphoid tissue, nodal MZL, and splenic MZL. Treatment of R/R MZL is similar to other indolent B-cell non-Hodgkin lymphoma (B-NHL) subtypes (e.g., follicular lymphoma [FL]), comprising rituximab-based immunochemotherapy regimens that achieve an ORR of 45-80%. Options after progression on rituximab-based regimens include the BTK inhibitor ibrutinib, which demonstrated an ORR of 46%, but a CR of only 3%, in its pivotal trial in R/R MZL. Overall, limited options are available after progression on current therapies, and a need remains for more effective treatments for R/R MZL.

Odronextamab is a bispecific antibody that binds CD20-expressing B-NHL cells and CD3 on T cells. In a Phase I study, odronextamab monotherapy showed antitumor activity and a manageable safety profile in a range of R/R B-NHL subtypes, including MZL (n=6; ORR 67%). In a Phase 2 study, odronextamab elicited an ORR of 82% and CR rate of 75% in patients with heavily pretreated R/R FL. These positive data in an indolent B-NHL subtype support evaluation of odronextamab in R/R MZL.

This is an open-label study of odronextamab in patients with R/R B-NHL comprising 5 disease-specific cohorts: FL, diffuse large B-cell lymphoma, mantle cell lymphoma, MZL, and other B-NHLs. Each cohort will be evaluated separately, and patients recruited from sites across North America, Europe, and Asia-Pacific regions. The MZL cohort is planned to include 78 patients, who will receive IV odronextamab monotherapy until disease progression or other protocol-defined reason for treatment discontinuation. Odronextamab is administered according to a step-up regimen during the first 21-day cycle (C), consisting of 0.7 mg split over C1 Day (D) 1 (0.2 mg) and C1D2 (0.5 mg), 4 mg split over C1D8 and C1D9, and 20 mg split over C1D15 and C1D16. The full 80 mg dose is given QW during C2 to C4, then 160 mg Q2W from C5 onwards. If a patient achieves a CR and has a durable response for months after initial determination of the CR, then dosing interval will be decreased from Q2W to Q4W.

Patients eligible for the MZL cohort will be ≥18 years of age; refractory to prior lines of systemic therapy; ECOG performance status ≤1; and have adequate bone marrow function and hepatic functions. Patients with prior allogeneic stem cell transplant or CAR T treatment will be excluded.

Primary endpoint is ORR (Lugano classification; assessed by independent central review). Key secondary endpoints include ORR (investigator evaluation); CR rate; progression-free survival; overall survival; duration of response; safety; pharmacokinetics; and patient-reported quality of life outcomes.

As of January 2023, 19 patients with MZL had enrolled.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

TABLE 40
Sequence Excluded from ST.26-Formatted Sequence Listing

| SEQ ID NO: | Sequence |
|---|---|
| 14 | GAS |

What is claimed is:

1. A dosing regimen for administering an anti-CD3 x anti-CD20 bispecific antibody to a subject to treat a B-cell malignancy, comprising:
administering an initial dose of 0.7 mg of the bispecific antibody to the subject, wherein the initial dose is split into a first dose fraction comprising 0.2 mg of the bispecific antibody and a second dose fraction comprising 0.5 mg of the bispecific antibody, wherein the first dose fraction is administered to the subject followed by the second dose fraction over two days during week 1 of the dosing regimen;
administering a first intermediate dose of 4 mg of the bispecific antibody to the subject, wherein the first intermediate dose is split into two equal fractions (a first fraction and a second fraction), each comprising 2 mg of the bispecific antibody, wherein the two fractions of the first intermediate dose are administered over two days during week 2 of the dosing regimen;
administering a second intermediate dose of 20 mg of the bispecific antibody to the subject, wherein the second intermediate dose is split into two equal fractions (a first fraction and a second fraction), each comprising 10 mg of the bispecific antibody, wherein the two fractions of the second intermediate dose are administered over two days during week 3 of the dosing regimen;
administering a full dose of the bispecific antibody to the subject weekly during weeks 4 to 12 of the dosing regimen; and
administering a maintenance dose of the bispecific antibody to the subject in week 14 of the dosing regimen,
wherein the bispecific antibody comprises a first antigen-binding region that binds human CD20 and a second antigen-binding region that binds human CD3, wherein the first antigen-binding region comprises three heavy chain complementarity determining regions, HCDR1, HCDR2 and HCDR3 comprising the amino acid sequences of SEQ ID NO: 7, 8 and 9, respectively, and three light chain complementarity determining regions LCDR1, LCDR2 and LCDR3 comprising the amino acid sequences of SEQ ID NO: 13, 14 and 15, respectively, and wherein the second antigen-binding region comprises three heavy chain complementarity determining regions, HCDR1, HCDR2 and HCDR3 comprising the amino acid sequences of SEQ ID NO: 10, 11 and 12, respectively, and three light chain complementarity determining regions LCDR1, LCDR2 and LCDR3 comprising the amino acid sequences of SEQ ID NO: 13, 14 and 15, respectively.

2. The dosing regimen of claim 1, wherein the full dose is administered to the subject as a single dose during weeks 4 to 12 of the dosing regimen.

3. The dosing regimen of claim 1, wherein if the subject experiences a grade 3 event of cytokine release syndrome when administered the initial dose, the first intermediate dose, or the second intermediate dose, then the full dose of the bispecific antibody administered to the subject during week 4 is split into two equal fractions and the two fractions of the full dose are administered over two days during week 4 of the dosing regimen, and wherein the full dose is administered to the subject as a single dose during weeks 5 to 12 of the dosing regimen.

4. The dosing regimen of any one of claims 1-3, wherein the maintenance dose is administered to the subject every two weeks beginning in week 14 of the dosing regimen.

5. The dosing regimen of any one of claims 1-4, wherein the maintenance dose is administered to the subject every four weeks or every eight weeks beginning in a subsequent week of the dosing regimen, wherein the subsequent week is at least week 36 of the dosing regimen.

6. The dosing regimen of any one of claims 1-5, wherein the second dose fraction of the initial dose is administered to the subject from 18 to 96 hours after the first dose fraction of the initial dose.

7. The dosing regimen of any one of claims 1-6, wherein the two fractions of the first intermediate dose are administered to the subject from 18 to 96 hours apart.

8. The dosing regimen of any one of claims 1-7, wherein the two fractions of the second intermediate dose are administered to the subject from 18 to 96 hours apart.

9. The dosing regimen of claim 3, wherein the two fractions of the full dose are administered to the subject from 18 to 96 hours apart.

10. The dosing regimen of any one of claims 1-5, wherein the two days are consecutive days.

11. The dosing regimen of any one of claims 1-5, wherein the two days are no more than three days apart.

12. The dosing regimen of any one of claims 1-11, wherein the B-cell malignancy is a B-cell non-Hodgkin lymphoma.

13. The dosing regimen of claim 12, wherein the B-cell malignancy is follicular lymphoma, diffuse large B-cell lymphoma, mantle cell lymphoma, or marginal zone lymphoma.

14. The dosing regimen of any one of claims 1-13, wherein the full dose of the bispecific antibody is from 80 mg to 320 mg.

15. The dosing regimen of any one of claims 1-14, wherein the maintenance dose of the bispecific antibody is from 160 mg to 320 mg.

16. The dosing regimen of any one of claims 1-15, wherein the cancer is follicular lymphoma, the full dose is 80 mg and the maintenance dose is 160 mg.

17. The dosing regimen of claim 16, wherein the follicular lymphoma is grade 1-3a.

18. The dosing regimen of any one of claims 1-15, wherein the cancer is diffuse large B-cell lymphoma, the full dose is 160 mg and the maintenance dose is 320 mg.

19. The dosing regimen of any one of claims 1-15, wherein the cancer is diffuse large B-cell lymphoma, the full dose is 320 mg and the maintenance dose is 320 mg.

20. The dosing regimen of claim 18 or 19, wherein the subject has failed prior CAR-T therapy.

21. The dosing regimen of any one of claims 1-15, wherein the cancer is mantle cell lymphoma, the full dose is 160 mg and the maintenance dose is 320 mg.

22. The dosing regimen of claim 21, wherein the subject has failed prior Bruton tyrosine kinase (BTK) inhibitor therapy.

23. The dosing regimen of any one of claims 1-15, wherein the cancer is marginal zone lymphoma, the full dose is 80 mg and the maintenance dose is 160 mg.

24. The dosing regimen of any one of claims 1-15, wherein the cancer is a non-Hodgkin lymphoma other than follicular lymphoma, diffuse large B-cell lymphoma, mantle cell lymphoma, or marginal zone lymphoma, the full dose is 160 mg and the maintenance dose is 320 mg.

25. The dosing regimen of any one of claims 1-15, wherein the cancer is an aggressive lymphoma, the full dose is 160 mg and the maintenance dose is 320 mg.

26. The dosing regimen of any one of claims 1-25, wherein: (a) the subject has documented CD20+ B-cell malignancy, with active disease not responsive to prior therapy, for whom no standard of care options exists, and for whom treatment with an anti-CD20 antibody may be appropriate; or (b) the subject has documented aggressive B-NHL, has relapsed within one year after frontline therapy and intends to proceed with autologous stem cell transplant (ASCT).

27. The dosing regimen of any one of claims 1-26, wherein the subject has relapsed or refractory disease.

28. The dosing regimen of any one of claims 1-27, wherein the subject has relapsed or is refractory to at least 2 prior lines of systemic therapy, including an anti-CD20 antibody and an alkylating agent.

29. The dosing regimen of any one of claims 1-28, wherein the subject is refractory to an anti-CD20 antibody in any line of prior therapy.

30. The dosing regimen of any one of claims 1-29, wherein the subject is double refractory to an alkylating agent and an anti-CD20 antibody in any line of prior therapy.

31. The dosing regimen of any one of claims 1-30, wherein the subject is a human aged 18 years.

32. The dosing regimen of any one of claims 1-31, wherein the bispecific antibody is administered intravenously.

33. A dosing regimen for administering an anti-CD3 x anti-CD20 bispecific antibody to a subject to treat a B-cell malignancy, comprising:
administering an initial dose of 1 mg or 2 mg of the bispecific antibody to the subject during week 1 of the dosing regimen;
administering a first intermediate dose of 10 mg or 26 mg of the bispecific antibody to the subject during week 2 of the dosing regimen;
administering a second intermediate dose of 50 mg or 100 mg of the bispecific antibody to the subject during week 3 of the dosing regimen; and
administering a full dose of the bispecific antibody to the subject during week 4 and during a subsequent week of the dosing regimen,
wherein the bispecific antibody comprises a first antigen-binding region that binds human CD20 and a second antigen-binding region that binds human CD3, wherein the first antigen-binding region comprises three heavy chain complementarity determining regions, HCDR1, HCDR2 and HCDR3 comprising the amino acid sequences of SEQ ID NO: 7, 8 and 9, respectively, and three light chain complementarity determining regions LCDR1, LCDR2 and LCDR3 comprising the amino acid sequences of SEQ ID NO: 13, 14 and 15, respectively, and wherein the second antigen-binding region comprises three heavy chain complementarity determining regions, HCDR1, HCDR2 and HCDR3 comprising the amino acid sequences of SEQ ID NO: 10, 11 and 12, respectively, and three light chain complementarity determining regions LCDR1, LCDR2 and LCDR3 comprising the amino acid sequences of SEQ ID NO: 13, 14 and 15, respectively.

34. The method of claim 33, wherein the initial dose is 2 mg.

35. The method of claim 33 or 34, wherein the first intermediate dose is 26 mg.

36. The method of any one of claims 33-35, wherein the second intermediate dose is 100 mg.

37. The method of any one of claims 33-36, wherein the full dose is 200 mg, 400 mg or 600 mg.

38. The method of claim 37, wherein the full dose is 400 mg.

39. The method of claim 37, wherein the full dose is 600 mg.

40. The method of any one of claims 33-39, wherein the full dose is administered to the subject once every three weeks.

41. The method of any one of claims 33-39, wherein the full dose is administered to the subject weekly.

42. The method of claim 41, wherein the full dose is administered to the subject weekly for three weeks, and then the full dose is administered to the subject once every three weeks.

43. The dosing regimen of any one of claims 33-42, wherein the subject has relapsed or refractory disease.

44. The dosing regimen of any one of claims 33-43, wherein the subject is refractory to an anti-CD20 antibody in any line of prior therapy.

45. The dosing regimen of any one of claims 33-44, wherein the B-cell malignancy is follicular lymphoma.

46. The dosing regimen of any one of claims 33-44, wherein the B-cell malignancy is diffuse large B-cell lymphoma.

47. The dosing regimen of any one of claims 33-46, wherein the subject is a human aged 18 years.

48. The method of any one of claims 33-47, wherein the bispecific antibody is administered subcutaneously.

49. A dosing regimen for administering an anti-CD3 x anti-CD20 bispecific antibody to a subject to treat a B-cell malignancy, comprising:
administering an initial dose of the bispecific antibody to the subject, wherein the initial dose is split into a first dose fraction of the bispecific antibody and a second dose fraction of the bispecific antibody, wherein the first dose fraction is administered to the subject followed by the second dose fraction over two days during week 1 of the dosing regimen;
administering a first intermediate dose of the bispecific antibody to the subject, wherein the first intermediate dose is split into two equal fractions (a first fraction and a second fraction) of the bispecific antibody, wherein the two fractions of the first intermediate dose are administered over two days during week 2 of the dosing regimen;
administering a second intermediate dose of the bispecific antibody to the subject, wherein the second intermediate dose is split into two equal fractions (a first fraction and a second fraction) of the bispecific antibody, wherein the two fractions of the second intermediate dose are administered over two days during week 3 of the dosing regimen;
administering a full dose of the bispecific antibody to the subject weekly during weeks 4 to 12 of the dosing regimen; and
administering a maintenance dose of the bispecific antibody to the subject in week 14 of the dosing regimen,
wherein the initial dose, the first intermediate dose, the second intermediate dose, the full dose and the maintenance dose is dependent on the weight of subject:
(i) if the subject has a weight of from 40 kg to 164 kg, the initial dose is 0.7 mg, the first dose fraction of the initial dose is 0.2 mg, the second dose fraction of the initial dose is 0.5 mg, the first intermediate dose is 4 mg, the two equal fractions of the first intermediate dose each comprise 2 mg, the second intermediate dose is 20 mg, the two equal fractions of the second intermediate dose each comprise 10 mg, the full dose is 160 mg, and the maintenance dose is 320 mg;
(ii) if the subject has a weight of from 20 kg to 39 kg, the initial dose is 0.4 mg, the first dose fraction of the initial dose is 0.1 mg, the second dose fraction of the initial dose is 0.3 mg, the first intermediate dose is 2 mg, the two equal fractions of the first intermediate dose each comprise 1 mg, the second intermediate dose is 12 mg, the two equal fractions of the second intermediate dose each comprise 6 mg, the full dose is 90 mg, and the maintenance dose is 150 mg;
(iii) if the subject has a weight of from 10 kg to 19 kg, the initial dose is 0.3 mg, the first dose fraction of the initial dose is 0.1 mg, the second dose fraction of the initial dose is 0.2 mg, the first intermediate dose is 1.6 mg, the two equal fractions of the first intermediate dose each comprise 0.8 mg, the second intermediate dose is 8 mg, the two equal fractions of the second intermediate dose each comprise 4 mg, the full dose is 60 mg, and the maintenance dose is 100 mg; or
(iv) if the subject has a weight of from 6 kg to 9 kg, the initial dose is 0.24 mg, the first dose fraction of the initial dose is 0.07 mg, the second dose fraction of the initial dose is 0.17 mg, the first intermediate dose is 1.2 mg, the two equal fractions of the first intermediate dose each comprise 0.6 mg, the second intermediate dose is 6 mg, the two equal fractions of the second intermediate dose each comprise 3 mg, the full dose is 45 mg, and the maintenance dose is 75 mg; and
wherein the bispecific antibody comprises a first antigen-binding region that binds human CD20 and a second antigen-binding region that binds human CD3, wherein the first antigen-binding region comprises three heavy chain complementarity determining regions, HCDR1, HCDR2 and HCDR3 comprising the amino acid sequences of SEQ ID NO: 7, 8 and 9, respectively, and three light chain complementarity determining regions LCDR1, LCDR2 and LCDR3 comprising the amino acid sequences of SEQ ID NO: 13, 14 and 15, respectively, and wherein the second antigen-binding region comprises three heavy chain complementarity determining regions, HCDR1, HCDR2 and HCDR3 comprising the amino acid sequences of SEQ ID NO: 10, 11 and 12, respectively, and three light chain complementarity determining regions LCDR1, LCDR2 and LCDR3 comprising the amino acid sequences of SEQ ID NO: 13, 14 and 15, respectively.

50. The dosing regimen of claim 49, wherein the full dose is administered to the subject as a single dose during weeks 4 to 12 of the dosing regimen.

51. The dosing regimen of claim 49 or 50, wherein the maintenance dose is administered to the subject every two weeks beginning in week 14 of the dosing regimen.

52. The dosing regimen of any one of claims 49-51, wherein the second dose fraction of the initial dose is administered to the subject from 18 to 96 hours after the first dose fraction of the initial dose.

53. The dosing regimen of any one of claims 49-52, wherein the two fractions of the first intermediate dose are administered to the subject from 18 to 96 hours apart.

54. The dosing regimen of any one of claims 49-53, wherein the two fractions of the second intermediate dose are administered to the subject from 18 to 96 hours apart.

55. The dosing regimen of any one of claims 49-51, wherein the two days are consecutive days.

56. The dosing regimen of any one of claims 49-51, wherein the two days are no more than three days apart.

57. The dosing regimen of any one of claims 49-56, wherein the B-cell malignancy is a B-cell non-Hodgkin lymphoma.

58. The dosing regimen of claim 57, wherein the B-cell malignancy is follicular lymphoma, diffuse large B-cell lymphoma, mantle cell lymphoma, or marginal zone lymphoma.

59. The dosing regimen of any one of claims 49-58, wherein the bispecific antibody is administered to the subject intravenously.

60. The dosing regimen of any one of claims 49-59, wherein the subject is a human aged<18 years.

61. A dosing regimen for administering an anti-CD3 x anti-CD20 bispecific antibody to a subject to treat follicular lymphoma, comprising:
administering an initial dose of 0.7 mg of the bispecific antibody to the subject, wherein the initial dose is split into a first dose fraction comprising 0.2 mg of the bispecific antibody and a second dose fraction comprising 0.5 mg of the bispecific antibody, wherein the first dose fraction is administered to the subject followed by the second dose fraction over two days during week 1 of the dosing regimen;
administering a first intermediate dose of 4 mg of the bispecific antibody to the subject, wherein the first intermediate dose is split into two equal fractions (a first fraction and a second fraction), each comprising 2 mg of the bispecific antibody, wherein the two fractions of the first intermediate dose are administered over two days during week 2 of the dosing regimen;
administering a second intermediate dose of 20 mg of the bispecific antibody to the subject, wherein the second intermediate dose is split into two equal fractions (a first fraction and a second fraction), each comprising 10 mg of the bispecific antibody, wherein the two fractions of the second intermediate dose are administered over two days during week 3 of the dosing regimen;
administering a full dose of 40 mg or 80 mg of the bispecific antibody to the subject weekly during weeks 4 to 12 of the dosing regimen; and
administering a maintenance dose of 80 mg or 160 mg of the bispecific antibody to the subject in week 14 of the dosing regimen,
wherein the bispecific antibody comprises a first antigen-binding region that binds human CD20 and a second antigen-binding region that binds human CD3, wherein the first antigen-binding region comprises three heavy chain complementarity determining regions, HCDR1, HCDR2 and HCDR3 comprising the amino acid sequences of SEQ ID NO: 7, 8 and 9, respectively, and three light chain complementarity determining regions LCDR1, LCDR2 and LCDR3 comprising the amino acid sequences of SEQ ID NO: 13, 14 and 15, respectively, and wherein the second antigen-binding region comprises three heavy chain complementarity determining regions, HCDR1, HCDR2 and HCDR3 comprising the amino acid sequences of SEQ ID NO: 10, 11 and 12, respectively, and three light chain complementarity determining regions LCDR1, LCDR2 and LCDR3 comprising the amino acid sequences of SEQ ID NO: 13, 14 and 15, respectively.

62. A dosing regimen for administering an anti-CD3 x anti-CD20 bispecific antibody to a subject to treat diffuse large B-cell lymphoma, comprising:
administering an initial dose of 0.7 mg of the bispecific antibody to the subject, wherein the initial dose is split into a first dose fraction comprising 0.2 mg of the bispecific antibody and a second dose fraction comprising 0.5 mg of the bispecific antibody, wherein the first dose fraction is administered to the subject followed by the second dose fraction over two days during week 1 of the dosing regimen;
administering a first intermediate dose of 4 mg of the bispecific antibody to the subject, wherein the first intermediate dose is split into two equal fractions (a first fraction and a second fraction), each comprising 2 mg of the bispecific antibody, wherein the two fractions of the first intermediate dose are administered over two days during week 2 of the dosing regimen;
administering a second intermediate dose of 20 mg of the bispecific antibody to the subject, wherein the second intermediate dose is split into two equal fractions (a first fraction and a second fraction), each comprising 10 mg of the bispecific antibody, wherein the two fractions of the second intermediate dose are administered over two days during week 3 of the dosing regimen;
administering a full dose of 80 mg or 160 mg of the bispecific antibody to the subject weekly during weeks 4 to 12 of the dosing regimen; and
administering a maintenance dose of 160 mg or 320 mg of the bispecific antibody to the subject in week 13 or week 14 of the dosing regimen,
wherein the bispecific antibody comprises a first antigen-binding region that binds human CD20 and a second antigen-binding region that binds human CD3, wherein the first antigen-binding region comprises three heavy chain complementarity determining regions, HCDR1, HCDR2 and HCDR3 comprising the amino acid sequences of SEQ ID NO: 7, 8 and 9, respectively, and three light chain complementarity determining regions LCDR1, LCDR2 and LCDR3 comprising the amino acid sequences of SEQ ID NO: 13, 14 and 15, respectively, and wherein the second antigen-binding region comprises three heavy chain complementarity determining regions, HCDR1, HCDR2 and HCDR3 comprising the amino acid sequences of SEQ ID NO: 10, 11 and 12, respectively, and three light chain complementarity determining regions LCDR1, LCDR2 and LCDR3 comprising the amino acid sequences of SEQ ID NO: 13, 14 and 15, respectively.

63. The dosing regimen of claim 61 or 62, wherein the full dose is administered to the subject as a single dose during weeks 4 to 12 of the dosing regimen.

64. The dosing regimen of any one of claims 61-63, wherein the maintenance dose is administered to the subject every two weeks beginning in week 13 or week 14 of the dosing regimen.

65. The dosing regimen of any one of claims 61-63, wherein the maintenance dose is administered to the subject every four weeks or every eight weeks.

66. The dosing regimen of any one of claims 61 and 63-65, wherein the full dose is 40 mg.

67. The dosing regimen of any one of claims 61-65, wherein the full dose is 80 mg.

68. The dosing regimen of any one of claims 62-65, wherein the full dose is 160 mg.

69. The dosing regimen of any one of claims 61 and 63-67, wherein the maintenance dose is 80 mg.

70. The dosing regimen of any one of claims 61-68, wherein the maintenance dose is 160 mg.

71. The dosing regimen of any one of claims 62-65, 67 and 68, wherein the maintenance dose is 320 mg.

72. The dosing regimen of any one of claims 61-71, wherein the second dose fraction of the initial dose is administered to the subject from 18 to 96 hours after the first dose fraction of the initial dose.

73. The dosing regimen of any one of claims 61-72, wherein the two fractions of the first intermediate dose are administered to the subject from 18 to 96 hours apart.

74. The dosing regimen of any one of claims 61-73, wherein the two fractions of the second intermediate dose are administered to the subject from 18 to 96 hours apart.

75. The dosing regimen of any one of claims 61-74, wherein the dosing regimen further comprises administering a combination of cyclophosphamide, doxorubicin, vincristine, and prednisone (CHOP).

76. The dosing regimen of claim 75, wherein the CHOP is administered during a week preceding week 1 of the dosing regimen, and again during weeks 3, 6, 9. 12 and 15 of the dosing regimen.

77. The dosing regimen of claim 75 or 76, wherein the cyclophosphamide is administered at a dose of 750 mg/m², the doxorubicin is administered at a dose of 50 mg/m², the vincristine is administered at a dose of 1.4 mg/m², and the prednisone is administered at a dose of 100 mg, wherein the cyclophosphamide, doxorubicin and vincristine are administered once in the week preceding week 1 of the dosing regimen, and once in each of weeks 3, 6, 9, 12, 15 of the dosing regimen, and the prednisone is administered for five consecutive days in the week preceding week 1 of the dosing regimen, and for five consecutive days in each of weeks 3, 6, 9, 12 and 15 of the dosing regimen.

78. The dosing regimen of any one of claims 61-77, wherein the subject has relapsed or refractory disease.

79. The dosing regimen of any one of claims 61-78, wherein the subject is refractory to an anti-CD20 antibody in any line of prior therapy.

80. The dosing regimen of any one of claims 61-79, wherein the subject is double refractory to an alkylator and an anti-CD20 antibody.

81. The dosing regimen of any one of claims 61-80, wherein the subject received a prior autologous stem cell transplant.

82. The dosing regimen of any one of claims 61-77, wherein the subject has not been previously treated with a systemic anti-lymphoma therapy.

83. The dosing regimen of any one of claims 61-82, wherein the bispecific antibody is administered to the subject intravenously.

84. The dosing regimen of any one of claims 1-83, wherein the first antigen-binding region of the bispecific antibody comprises a heavy chain variable region (HCVR) comprising the amino acid sequence of SEQ ID NO: 4 and a light chain variable region (LCVR) comprising the amino acid sequence of SEQ ID NO: 6, and the second antigen-binding region of the bispecific antibody comprises a HCVR comprising the amino acid sequence of SEQ ID NO: 5 and a LCVR comprising the amino acid sequence of SEQ ID NO: 6.

85. The dosing regimen of any one of claims 1-74, wherein the bispecific antibody comprises a human IgG heavy chain constant region.

86. The dosing regimen of claim 85, wherein the human IgG heavy chain constant region is of isotype IgG1.

87. The dosing regimen of claim 85, wherein the human IgG heavy chain constant region is of isotype IgG4.

88. The dosing regimen of any one of claims 1-84, wherein the bispecific antibody comprises a first heavy chain constant region comprising the amino acid sequence of SEQ ID NO: 16 and a second heavy chain constant region comprising the amino acid sequence of SEQ ID NO: 17.

89. The dosing regimen of any one of claims 1-84, wherein the bispecific antibody comprises a first heavy chain constant region comprising the amino acid sequence of SEQ ID NO: 18 and a second heavy chain constant region comprising the amino acid sequence of SEQ ID NO: 19.

90. The dosing regimen of any one of claims 1-84, wherein the bispecific antibody comprises a first heavy chain comprising amino acid residues 1-452 of SEQ ID NO: 1 paired with a common light chain comprising the amino acid sequence of SEQ ID NO: 3, and a second heavy chain comprising amino acid residues 1-448 of SEQ ID NO: 2 paired with a common light chain comprising the amino acid sequence of SEQ ID NO: 3.

91. The dosing regimen of any one of claims 1-84, wherein the bispecific antibody comprises a first heavy chain comprising the amino acid sequence of SEQ ID NO: 1 paired with a common light chain comprising the amino acid sequence of SEQ ID NO: 3, and a second heavy chain comprising the amino acid sequence of SEQ ID NO: 2 paired with a common light chain comprising the amino acid sequence of SEQ ID NO: 3.

92. The dosing regimen of any one of claims 1-91, wherein the dosing regimen further comprises administering a dose of steroid to the subject: from 12 to 24 hours prior to the administration of the first dose fraction of the initial dose; from 12 to 24 hours prior to the administration of the first fraction of the first intermediate dose; and from 12 to 24 hours prior to the administration of the first fraction of the second intermediate dose.

93. The dosing regimen of claim 92, wherein if the first dose fraction of the initial dose and the second dose fraction of the initial dose are not administered to the subject on consecutive days, then the dosing regimen further comprises administering a dose of steroid to the subject from 12 to 24 hours prior to the administration of the second dose fraction of the initial dose of the bispecific antibody.

94. The dosing regimen of claim 92 or 93, wherein if the first fraction of the first intermediate dose and the second fraction of the first intermediate dose are not administered to the subject on consecutive days, then the dosing regimen further comprises administering a dose of steroid to the subject from 12 to 24 hours prior to the administration of the second fraction of the first intermediate dose of the bispecific antibody.

95. The dosing regimen of any one of claims 92-94, wherein if the first fraction of the second intermediate dose and the second fraction of the second intermediate dose are not administered to the subject on consecutive days, then the dosing regimen further comprises administering a dose of steroid to the subject from 12 to 24 hours prior to the administration of the second fraction of the second intermediate dose of the bispecific antibody.

96. The dosing regimen of any one of claims 92-95, wherein the dosing regimen further comprises:
administering a dose of steroid to the subject: from 1 to 3 hours prior to the administration of the first dose fraction of the initial dose; from 1 to 3 hours prior to the administration of the second dose fraction of the initial dose; and from 1 to 3 hours prior to the administration of each fraction of the first intermediate dose and the second intermediate dose, and
administering a dose of antihistamine to the subject: from 30 to 60 minutes prior to the administration of the first dose fraction of the initial dose; from 30 to 60 minutes prior to the administration of the second dose fraction of the initial dose; and from 30 to 60 minutes prior to the administration of each fraction of the first intermediate dose and the second intermediate dose.

97. The dosing regimen of claim 96, wherein the dosing regimen further comprises administering a dose of acetaminophen to the subject: from 30 to 60 minutes prior to the administration of the first dose fraction of the initial dose; from 30 to 60 minutes prior to the administration of the second dose fraction of the initial dose; and from 30 to 60 minutes prior to the administration of each fraction of the first intermediate dose and the second intermediate dose.

98. The dosing regimen of any one of claims 92-97, wherein the dosing regimen further comprises administering a dose of steroid to the subject: from 20 to 28 hours after the end of administration of the second dose fraction of the initial dose; from 20 to 28 hours after the end of administration of the second fraction of the first intermediate dose; and from 20 to 28 hours after the end of administration of the second fraction of the second intermediate dose.

99. The dosing regimen of any one of claims 92-98, wherein the dosing regimen further comprises:
administering a dose of steroid to the subject from 1 to 3 hours prior to the administration of the full dose during week 4 of the dosing regimen, and
administering a dose of antihistamine to the subject from 30 to 60 minutes prior to the administration of the full dose during week 4 of the dosing regimen.

100. The dosing regimen of claim 99, wherein the dosing regimen further comprises administering a dose of acetaminophen to the subject from 30 to 60 minutes prior to the administration of the full dose during week 4 of the dosing regimen.

101. The dosing regimen of claim 100, wherein the dosing regimen further comprises administering a dose of steroid to the subject from 20 to 28 hours after the end of the administration of the full dose during week 4 of the dosing regimen.

102. The dosing regimen of any one of claims 92-98, wherein if the subject experiences a grade 3 event of cytokine release syndrome when administered the initial dose, the first intermediate dose, or the second intermediate dose, then the full dose of the bispecific antibody administered to the subject during week 4 is split into two equal fractions (a first fraction and a second fraction) and the two fractions of the full dose are administered over two days during week 4 of the dosing regimen, and wherein the dosing regimen further comprises administering a dose of steroid to the subject from 12 to 24 hours prior to the administration of the first fraction of the full dose.

103. The dosing regimen of claim 102, wherein if the first fraction of the full dose and the second fraction of the full dose are not administered to the subject on consecutive days, then the dosing regimen further comprises administering a dose of steroid to the subject from 12 to 24 hours prior to the administration of the second fraction of the full dose of the bispecific antibody.

104. The dosing regimen of claim 102 or 103, wherein the dosing regimen further comprises:
administering a dose of steroid to the subject from 1 to 3 hours prior to the administration of the first fraction of the full dose, and
administering a dose of antihistamine to the subject from 30 to 60 minutes prior to the administration of the first fraction of the full dose.

105. The dosing regimen of claim 104, wherein the dosing regimen further comprises administering a dose of acetaminophen to the subject from 30 to 60 minutes prior to the administration of the first fraction of the full dose.

106. The dosing regimen of any one of claims 102-105, wherein the dosing regimen further comprises:
administering a dose of steroid to the subject from 1 to 3 hours prior to the administration of the full dose during week 5 of the dosing regimen, and
administering a dose of antihistamine to the subject from 30 to 60 minutes prior to the administration of the full dose during week 5 of the dosing regimen.

107. The dosing regimen of claim 106, wherein the dosing regimen further comprises administering a dose of acetaminophen to the subject from 30 to 60 minutes prior to the administration of the full dose during week 5 of the dosing regimen.

108. The dosing regimen of claim 107, wherein the dosing regimen further comprises administering a dose of steroid to the subject from 20 to 28 hours after the end of the administration of the full dose during week 5 of the dosing regimen.

109. The dosing regimen of any one of claims 92-108, wherein administering a dose of steroid, administering a dose of antihistamine, or administering a dose of acetaminophen comprises instructing the subject to ingest the dose of steroid, the dose of antihistamine, or the dose of acetaminophen, respectively.

110. The dosing regimen of any one of claims 92-108, wherein administering a dose of steroid, or administering a dose of antihistamine comprises intravenously administering the dose of steroid or the dose of antihistamine.

111. The dosing regimen of any one of claims 92-110, wherein the steroid is dexamethasone.

112. The dosing regimen of claim 111, wherein the dose of steroid is 20 mg.

113. The dosing regimen of any one of claims 96-112, wherein the antihistamine is diphenhydramine.

114. The dosing regimen of claim 113, wherein the dose of antihistamine is 25 mg.

115. The dosing regimen of any one of claims 97-114, wherein the dose of acetaminophen is 650 mg.

116. The dosing regimen of any one of claims 1-115, wherein the dosing regimen further comprises administration of an anti-IL-6 receptor antibody.

117. The dosing regimen of claim 116, wherein the anti-IL-6 receptor antibody is tocilizumab or sarilumab.

118. A method of treating a B-cell cancer in a subject, comprising:
selecting a subject diagnosed with a B-cell cancer; and
administering the bispecific antibody to the subject according to the dosing regimen of any one of claims 1-117.

119. The method of claim 118, wherein:
(a) the subject has been diagnosed with follicular lymphoma;
(b) the subject has been diagnosed with follicular lymphoma of grade 1-3a;
(c) the subject has been diagnosed with relapsed or refractory follicular lymphoma after at least 2 prior lines of systemic therapy;
(d) the subject has been diagnosed with follicular lymphoma and has not previously been treated with a systemic anti-lymphoma therapy;
(e) the subject has been diagnosed with follicular lymphoma, and the full dose is 80 mg; and/or
(f) the subject has been diagnosed with follicular lymphoma, and the maintenance dose is 160 mg or 320 mg.

120. The method of claim 118, wherein:
(a) the subject has been diagnosed with diffuse large B-cell lymphoma (DLBCL);
(b) the subject has been diagnosed with DLBCL, and wherein the DLBCL is de novo or is transformed from a lower grade neoplasm;
(c) the subject has been diagnosed with DLBCL and is refractory to at least 2 prior lines of systemic therapy;
(d) the subject has been diagnosed with relapsed or refractory DLBCL after at least 2 prior lines of systemic therapy including CAR-T therapy;
(e) the subject has been diagnosed with DLBCL and has not previously been treated with a systemic anti-lymphoma therapy;
(f) the subject has been diagnosed with DLBCL, and the full dose is 160 mg; and/or
(g) the subject has been diagnosed with DLBCL, and the maintenance dose is 320 mg.

121. A pharmaceutical kit comprising (i) a container containing the bispecific antibody, and (ii) a label including instructions to administer the bispecific antibody according to the dosing regimen of any one of claims 1-91.

122. The pharmaceutical kit of claim 121, wherein the label further includes instructions to administer the steroid and antihistamine according to the dosing regimen of any one of claims 92-115 and/or the anti-IL-6 receptor antibody of claim 116 or 117.


SEQUENCE LISTING (19 sequences)

SEQ ID NO: 1 - protein, 453 aa, synthetic construct
  REGION 1..453: note = Synthetic
EVQLVESGGGLVQPGRSLRLSCVASGFTFNDYAMHWVRQAPGKGLEWVSVISWNSDSIGYADSVKGRFTISRDNAKNSLYLQMHSLRAEDTALYYCAKDNHYGSGSYYYYQYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 2 - protein, 449 aa, synthetic construct
  REGION 1..449: note = Synthetic
EVQLVESGGGLVQPGRSLRLSCAASGFTFDDYTMHWVRQAPGKGLEWVSGISWNSGSIGYADSVKGRFTISRDNAKKSLYLQMNSLRAEDTALYYCAKDNSGYGHYYYGMDVWGQGTTVTVASASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNRFTQKSLSLSLGK

SEQ ID NO: 3 - protein, 214 aa, synthetic construct
  REGION 1..214: note = Synthetic
EIVMTQSPATLSVSPGERATLSCRASQSVSSNLAWYQQKPGQAPRLLIYGASTRATGIPARFSGSGSGTEFTLTISSLQSEDFAVYYCQHYINWPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 127 aa, synthetic construct
  REGION 1..127: note = Synthetic
EVQLVESGGGLVQPGRSLRLSCVASGFTFNDYAMHWVRQAPGKGLEWVSVISWNSDSIGYADSVKGRFTISRDNAKNSLYLQMHSLRAEDTALYYCAKDNHYGSGSYYYYQYGMDVWGQGTTVTVSS

SEQ ID NO: 5 - protein, 123 aa, synthetic construct
  REGION 1..123: note = Synthetic
EVQLVESGGGLVQPGRSLRLSCAASGFTFDDYTMHWVRQAPGKGLEWVSGISWNSGSIGYADSVKGRFTISRDNAKKSLYLQMNSLRAEDTALYYCAKDNSGYGHYYYGMDVWGQGTTVTVAS

SEQ ID NO: 6 - protein, 108 aa, synthetic construct
  REGION 1..108: note = Synthetic
EIVMTQSPATLSVSPGERATLSCRASQSVSSNLAWYQQKPGQAPRLLIYGASTRATGIPARFSGSGSGTEFTLTISSLQSEDFAVYYCQHYINWPLTFGGGTKVEIKR

SEQ ID NO: 7 - protein, 8 aa, synthetic construct
  REGION 1..8: note = Synthetic
GFTFNDYA

SEQ ID NO: 8 - protein, 8 aa, synthetic construct
  REGION 1..8: note = Synthetic
ISWNSDSI

SEQ ID NO: 9 - protein, 20 aa, synthetic construct
  REGION 1..20: note = Synthetic
AKDNHYGSGSYYYYQYGMDV

SEQ ID NO: 10 - protein, 8 aa, synthetic construct
  REGION 1..8: note = Synthetic
GFTFDDYT

SEQ ID NO: 11 - protein, 8 aa, synthetic construct
  REGION 1..8: note = Synthetic
ISWNSGSI

SEQ ID NO: 12 - protein, 16 aa, synthetic construct
  REGION 1..16: note = Synthetic
AKDNSGYGHYYYGMDV

SEQ ID NO: 13 - protein, 6 aa, synthetic construct
  REGION 1..6: note = Synthetic
QSVSSN

SEQ ID NO: 14 - skipped

SEQ ID NO: 15 - protein, 9 aa, synthetic construct
  REGION 1..9: note = Synthetic
QHYINWPLT

SEQ ID NO: 16 - protein, 326 aa, synthetic construct
  REGION 1..326: note = Synthetic
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 17 - protein, 326 aa, synthetic construct
  REGION 1..326: note = Synthetic
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNRFTQKSLSLSLGK

SEQ ID NO: 18 - protein, 329 aa, synthetic construct
  REGION 1..329: note = Synthetic
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 329 aa, synthetic construct
  REGION 1..329: note = Synthetic
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNRFTQKSLSLSPGK